(19) United States
(12) Patent Application Publication
(10) Pub. No.: US 2017/0002405 A1
(43) Pub. Date: Jan. 5, 2017

(54) METHODS AND KITS FOR SIMULTANEOUSLY DETECTING GENE OR PROTEIN EXPRESSION IN A PLURALITY OF SAMPLE TYPES USING SELF-ASSEMBLING FLUORESCENT BARCODE NANOREPORTERS

(71) Applicant: NanoString Technologies, Inc., Seattle, WA (US)

(72) Inventors: Chris MERRITT, Seattle, WA (US); Philippa J. WEBSTER, Seattle, WA (US)

(21) Appl. No.: 15/197,980

(22) Filed: Jun. 30, 2016

Related U.S. Application Data
(60) Provisional application No. 62/186,818, filed on Jun. 30, 2015.

(51) Int. Cl. C12Q 1/68

(52) U.S. Cl. CPC C12Q 1/6832; C12Q 1/6825

(57) ABSTRACT

The present invention relates to, among other things, probes, compositions, methods, and kits for simultaneously detecting nucleic acids or proteins in a plurality of samples.

### CROSS-REFERENCE TO RELATED APPLICATIONS

This application claims priority to and the benefit of U.S. Provisional Application No. 62/186,818, filed Jun. 30, 2015, the contents of which are incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Jun. 29, 2016, is named NATE-027_ST25.txt and is 170,565 bytes in size.

### BACKGROUND OF THE INVENTION

Current methods for detecting nucleic acid or protein targets in a plurality of samples, in which the identity and quantity of each target for each sample is determined, are time consuming and costly. There exists a need for a new probes, compositions, methods, and kits for simultaneously detecting nucleic acid or protein targets in a plurality of samples.

### SUMMARY OF THE INVENTION

The present invention relates to a new probes, compositions, methods, and kits for simultaneously detecting nucleic acid or protein targets in a plurality of samples.

A first aspect of the present invention relates to a single-stranded nucleic acid probe including at least three regions: at least a first region capable of binding to a target nucleic acid in a sample, at least a second region capable of binding to at least a first plurality of labeled single-stranded oligonucleotides, in which the first plurality of labeled single-stranded oligonucleotides identifies the target nucleic acid in the sample, and at least a third region capable of binding to at least a second plurality of labeled single-stranded oligonucleotides, in which the second plurality of labeled single-stranded oligonucleotides identifies the sample.

In embodiments of this aspect or any other aspect or embodiment disclosed herein, a target nucleic acid is a synthetic oligonucleotide or is obtained from a biological sample. The second region may include at least one position (e.g., two, three, four, five, six, seven, eight, nine, ten, or more) for binding to the first pluralities of labeled single-stranded oligonucleotides; the first plurality of labeled single-stranded oligonucleotides may include or be complementary to one or more of SEQ ID NO: 1 to SEQ ID NO: 808. The third region may include at least one position (e.g., two, three, four, five, six, seven, eight, nine, ten, or more) for binding to the second pluralities of labeled single-stranded oligonucleotides; the second plurality of labeled single-stranded oligonucleotides may include or be complementary to one or more of SEQ ID NO: 1 to SEQ ID NO: 808. In embodying single-stranded probes, a first plurality of labeled single-stranded oligonucleotides is not identical to a second plurality of labeled single-stranded oligonucleotides.

In any aspect or embodiment of the present invention, there is no upper limit to the number of positions present in a probe's second region and/or in the probe's third region. Additionally, in any aspect or embodiment of the present invention, there is no limit to the number of positions in a second region that can be combined with the number of positions for a third region. More specifically, a first probe may include a second region having one, two, three, four, five, six, seven, eight, nine ten, or more positions and a third region having one, two, three, four, five, six, seven, eight, nine, ten, or more positions. As non-limiting embodiments, a probe may include a second region having two, three, four, five, six, seven, eight, nine, ten, or more positions and a third region having two positions; a probe may include a second region having two, three, four, five, six, seven, eight, nine, ten, or more positions and a third region having three positions; a probe may include a second region having two, three, four, five, six, seven, eight, nine, ten, or more positions and a third region having four positions; a probe may include a second region having two, three, four, five, six, seven, eight, nine, ten, or more positions and a third region having five positions; or a probe may include a second region having two, three, four, five, six, seven, eight, nine, ten, or more positions and a third region having six positions.

The labeled single-stranded oligonucleotide may include deoxyribonucleotides, embodiments of which may have melting/hybridization temperatures of between about 65° C. and about 85° C., e.g., about 80° C. In embodiments, the label monomer of a labeled single-stranded oligonucleotide may be a fluorochrome, quantum dot, dye, enzyme, nanoparticle, chemiluminescent marker, biotin, or another monomer that can be detected directly or indirectly. In embodiments, a label monomer of one position is spectrally or spatially distinguishable from a label monomer of another position, within a region and/or between regions. In embodiments, a label monomer at a position of the second region that is adjacent to a position of the third region differs from a label monomer at the position of the third region that is adjacent to the position of the second region.

In any embodiment or aspect of the present invention, a single-stranded oligonucleotide may lack a label monomer. Thus, a position of a second region and/or a third position may be hybridized with at least one oligonucleotide lacking a detectable label. In these embodiments, a probe will have a “dark spot” adjacent to a position having a detectable signal. The term “dark spot” refers to a lack of signal from a label attachment site on a probe. Dark spots add more coding permutations and generate greater reporter diversity in a population of probes.

In any embodiment or aspect of the present invention, a single-stranded nucleic acid probe may comprise a single-stranded or double-stranded RNA, DNA, PNA, or other polynucleotide analogue spacer between its first region and its second region and/or between its first region and its third region. The spacer may be double-stranded DNA. The spacer may have similar mechanical properties as the probe's backbone. The spacer may be of any length, e.g., 1 to 100 nucleotides, e.g., 2 to 50 nucleotides. The spacer can be shorter than 20 nucleotides or longer than 40 nucleotides and it can be 20 to 40 nucleotides long. Non-limiting examples of a spacer includes the sequences covered by SEQ ID NO: 809 to SEQ ID NO: 813.

In any embodiment or aspect of the present invention, a probe may comprise at least one affinity moiety. The at least one affinity moiety may be attached to the probe by covalent or non-covalent means. Various affinity moieties appropriate for purification and/or for immobilization are known in the art. Preferably, the affinity moiety is biotin, avidin, or streptavidin. Other affinity tags are recognized by specific binding partners and thus facilitate isolation and immobilization by affinity binding to the binding partner, which can be immobilized onto a solid support.

A second aspect of the present invention relates to a composition including at least two single-stranded nucleic acid probes. The at least a first single-stranded nucleic acid probe includes at least three regions: at least a first region capable of binding to a first sequence of a target nucleic acid in a sample, at least a second region capable of binding to at least a first plurality of labeled single-stranded oligonucleotides, in which the first plurality of labeled single-stranded oligonucleotides identifies the target nucleic acid in the sample, and at least a third region capable of binding to at least a second plurality of labeled single-stranded oligonucleotides, in which the second plurality of labeled single-stranded oligonucleotides identifies the sample. The at least a second single-stranded nucleic acid probe includes at least two regions: at least a first region capable of binding to a second sequence of the target nucleic acid in a sample, in which the first and the second sequences of the target nucleic acid are different or to a second target nucleic acid and at least a second region including at least one affinity moiety (e.g., biotin, avidin, and streptavidin).

A third aspect of the present invention relates to a composition including a plurality of single-stranded nucleic acid probes. Each single-stranded nucleic acid probe includes at least three regions: at least a first region capable of binding to a target nucleic acid in a sample, at least a second region capable of binding to a first plurality of labeled single-stranded oligonucleotides, in which the first plurality of labeled single-stranded oligonucleotides identifies the target nucleic acid in the sample, and at least a third region capable of binding to a second plurality of labeled single-stranded oligonucleotides, in which the second plurality of labeled single-stranded oligonucleotides identifies the sample. In embodiments, the plurality of single-stranded nucleic acid probes are capable of binding to different target nucleic acids obtained from the same sample or the plurality of single-stranded nucleic acid probes are capable of binding to the same target nucleic acid obtained from different samples.

A fourth aspect of the present invention relates to a method for simultaneously detecting a target nucleic acid in at least two sample: The method includes steps of: (1) contacting a first sample with one or more first single-stranded nucleic acid probes capable of identifying a first target nucleic acid and the first sample, (2) contacting the first sample with a first plurality of labeled single-stranded oligonucleotides capable of binding to the first single-stranded nucleic acid probes that can identify the first target nucleic acid, (3) contacting the first sample with a second plurality of labeled single-stranded oligonucleotides capable of binding to the first single-stranded nucleic acid probes that can identify the first sample, (4) contacting at least a second sample with one or more at least second single-stranded nucleic acid probes capable of identifying the first target nucleic acid and the at least second sample, (5) contacting the at least second sample with the first plurality of labeled single-stranded oligonucleotides capable of binding to the at least second single-stranded nucleic acid probes that can identify the first target nucleic acid, (6) contacting the at least second sample with at least a third plurality of labeled single-stranded oligonucleotides capable of binding to the at least second single-stranded nucleic acid probes that can identify the at least second sample, (7) pooling the sample of step (3) and the sample of step (6) to form a combined sample, and (8) detecting the first and the at least second single-stranded nucleic acid probes in the combined sample, thereby simultaneously detecting the target nucleic acid in at least two samples. In embodiments, the first sample and the at least second sample are different. The method may further include embodiments of contacting the first and at least second sample with at least a third single-stranded nucleic acid probe comprising at least two regions: at least a first region capable of binding to a second sequence of the first target nucleic acid in a sample, wherein the first and the second sequences of the first target nucleic acid are different or capable of binding to a second target nucleic acid and at least a second region comprising at least one affinity moiety.

A fifth aspect of the present invention relates to a method for simultaneously detecting a target nucleic acid in at least two samples. The method includes steps of: (1) contacting a first sample with one or more first single-stranded nucleic acid probes capable of identifying a first target nucleic acid and the first sample to form one or more first complexes, in which the first single-stranded nucleic acid probes comprise a first plurality of labeled single-stranded oligonucleotides that can identify the first target nucleic acid, (2) contacting the one or more first complexes with a second plurality of labeled single-stranded oligonucleotides capable of binding to the first single-stranded nucleic acid probes that can identify the first sample, (3) contacting at least a second sample with one or more at least second single-stranded nucleic acid probes capable of identifying the first target nucleic acid and the at least second sample to form one or more second complexes, in which the at least second single-stranded nucleic acid probes comprise the first plurality of labeled single-stranded oligonucleotides that can identify the first target nucleic acid, (4) contacting the one or more second complexes with at least a third plurality of labeled single-stranded oligonucleotides capable of binding to the at least second single-stranded nucleic acid probes that can identify the at least second sample, in which the first sample and the at least second sample are different, (5) pooling the sample of step (2) and the sample of step (4) to form a combined sample, and (6) detecting the first and the at least second single-stranded nucleic acid probes in the combined sample, thereby simultaneously detecting the target nucleic acid in at least two samples.

A sixth aspect of the present invention relates to a method for simultaneously detecting a target nucleic acid in at least two samples. The method includes steps of: (1) contacting a first sample with one or more first single-stranded nucleic acid probes capable of identifying a first target nucleic acid and the first sample, in which the first single-stranded nucleic acid probes comprise a first plurality of labeled single-stranded oligonucleotides that can identify the first target nucleic acid and a second plurality of labeled single-stranded oligonucleotides that can identify the first sample, (2) contacting at least a second sample with one or more at least second single-stranded nucleic acid probes capable of identifying the first target nucleic acid and the at least second sample, in which the at least second single-stranded nucleic acid probes comprise the first plurality of labeled single-stranded oligonucleotides that can identify the first target nucleic acid and at least a third plurality of labeled single-stranded oligonucleotides that can identify the at least second sample, (3) pooling the sample of step (1) and the sample of step (2) to form a combined sample, and (4) detecting the first and the at least second single-stranded nucleic acid probes in the combined sample, thereby simultaneously detecting the target nucleic acid in at least two samples. In embodiments of this aspect, the first sample and the at least second sample are different.

A seventh aspect of the present invention relates to a method for simultaneously detecting a target nucleic acid in at least two samples. The method includes steps of: (1) contacting one or more first single-stranded nucleic acid probes capable of identifying a first target nucleic acid and the first sample with a second plurality of labeled single-stranded oligonucleotides capable of binding to the first single-stranded nucleic acid probes that can identify the first sample to form one or more first complexes, in which the first single-stranded nucleic acid probes comprise a first plurality of labeled single-stranded oligonucleotides that can identify the first target nucleic acid, (2) contacting the one or more first complexes with the first sample, (3) contacting one or more at least second single-stranded nucleic acid probes capable of identifying the first target nucleic acid and the at least second sample with at least a third plurality of labeled single-stranded oligonucleotides capable of binding to the at least second single-stranded nucleic acid probes that can identify the at least second sample to form one or more second complexes, in which the second single-stranded nucleic acid probes comprise the first plurality of labeled single-stranded oligonucleotides that can identify the first target nucleic acid, (4) contacting the one or more second complexes with at least a second sample, (5) pooling the sample of step (2) and the sample of step (4) to form a combined sample, and (6) detecting the first and the at least second single-stranded nucleic acid probes in the combined sample, thereby simultaneously detecting the target nucleic acid in at least two samples. In embodiments of this aspect, the first sample and the at least second sample are different.

An eighth aspect of the present invention relates to a kit including at least three containers. A first container includes a plurality of single-stranded nucleic acid probes, each single-stranded nucleic acid probe comprising at least three regions: at least a first region capable of binding to a target nucleic acid, at least a second region capable of binding to a first plurality of labeled single-stranded oligonucleotides, in which the first plurality of labeled single-stranded oligonucleotides identifies the target nucleic acid, and at least a third region capable of binding to at least a second or third plurality of labeled single-stranded oligonucleotides. In embodiments, the first container further includes the first plurality of labeled single-stranded oligonucleotides. A second container includes the second plurality of labeled single-stranded oligonucleotides that can identify the first sample. The at least third container includes at least the third plurality of labeled single-stranded oligonucleotides that can identify the at least second sample. In embodiments, the kit may further include a second single-stranded nucleic acid probe or a plurality of second single-stranded probes each probe including at least two regions: at least a first region capable of binding to a second sequence of the first target nucleic acid in a sample, wherein the first and the second sequences of the first target nucleic acid are different or capable of binding to a second target nucleic acid and at least a second region comprising at least one affinity moiety.

A ninth aspect of the present invention relates to a kit comprising at least four containers. A first container includes a plurality of single-stranded nucleic acid probes, each single-stranded nucleic acid probe comprising at least three regions: at least a first region capable of binding to a target nucleic acid, at least a second region capable of binding to a first plurality of labeled single-stranded oligonucleotides, in which the first plurality of labeled single-stranded oligonucleotides identifies the target nucleic acid, and at least a third region capable of binding to at least a second or third plurality of labeled single-stranded oligonucleotides. A second container includes the first plurality of labeled single-stranded oligonucleotides. A third container includes the second plurality of labeled single-stranded oligonucleotides that can identify the first sample. The at least fourth container includes at least the third plurality of labeled single-stranded oligonucleotides that can identify the at least second sample.

A tenth aspect of the present invention relates to a kit including at least two containers. A first container includes a plurality of single-stranded nucleic acid probes, each single-stranded nucleic acid probe comprising at least three regions: at least a first region capable of binding to a target nucleic acid, at least a second region capable of binding to a first plurality of labeled single-stranded oligonucleotides, in which the first plurality of labeled single-stranded oligonucleotides identifies the target nucleic acid, and at least a third region capable of binding to at least a second or third plurality of labeled single-stranded oligonucleotides. In embodiments, the first container further includes the first plurality of labeled single-stranded oligonucleotides and the second plurality of labeled single-stranded oligonucleotides that can identify a first sample. In embodiments, the at least second container includes the plurality of single-stranded nucleic acid probes and the first plurality of labeled single-stranded oligonucleotides, and the at least third plurality of labeled single-stranded oligonucleotides that can identify at least a second sample.

An eleventh aspect of the present invention relates to probes, compositions, kits, and methods including a single-stranded nucleic acid probe having at least two regions: at least a first region capable of binding to a target nucleic acid in a sample and at least a second region capable of binding to at least a plurality of labeled single-stranded oligonucleotides, in which the plurality of labeled single-stranded oligonucleotides identifies the sample. In embodiments, the second region may include at least one position (e.g., two, three, four, five, six, seven, eight, nine ten, or more) for binding to the pluralities of labeled single-stranded oligonucleotides; the pluralities of labeled single-stranded oligonucleotides may include or be complementary to one or more of SEQ ID NO: 1 to SEQ ID NO: 808. There is no upper limit to the number of positions present in a probe's second region.

Any of the above aspects or embodiments can be adapted for use in a twelfth aspect of the present invention, which relates to detecting protein targets in a plurality of samples. This twelfth aspect extends the prior aspects by further including at least one first protein probe specific for at least one target protein in a sample. The at least one first protein probe includes a first region capable of binding to target protein in a sample and a second region including a partially double-stranded nucleic acid or including a single-stranded nucleic acid. The second region of the protein probe can include a linker, e.g., a photocleavable linker, which when cleaved, can release a portion of the second region from the first region. In the twelfth aspect, a single-stranded nucleic acid probe including at least three regions has a first region capable of binding to a target nucleic acid in which the target nucleic acid is a portion of the first protein probe's second region. In embodiments, the twelfth aspect may further include at least one second protein probe specific for the at least one target protein in a sample, which includes a first region capable of binding to target protein in a sample and a second region including a capture region or a matrix. In embodiments, a protein probe's first region capable of binding to a target protein in a sample may be an antibody, a peptide, an aptamer, or a peptoid. An antibody can be obtained from a variety of sources, including but not limited to polyclonal antibody, monoclonal antibody, monospecific antibody, recombinantly expressed antibody, humanized antibody, plantibodies, and the like. Thus, in any embodiment or aspect of the present invention, a target nucleic acid in a sample may be a portion of a first protein probe that is released from or present in the first protein probe. Such first protein probes include a first region capable of binding to first target protein in a sample and a second region including a partially double-stranded nucleic acid or including a single-stranded nucleic acid. The partially double-stranded nucleic acid or the single-stranded nucleic acid is released from a first protein probe.

Any aspect or embodiment described herein can be combined with any other aspect or embodiment as disclosed herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1: Shows two exemplary Target- and Sample-specific probes. The target-identifying and the sample-identifying regions are shown. The two probes detect the same target; however, the top probe further identifies sample 1 as a source of the target nucleic acid whereas the bottom probe instead identifies sample 2.

FIG. 2: Shows a first type of composition of the present invention. Here, a first probe (a Target- and Sample-specific probe) and a second probe (a Capture probe) bind directly to a target nucleic acid which is obtained from a biological sample.

FIG. 3: Shows a second type of composition of the present invention. Here, a first probe (a Target- and Sample-specific probe) and a second probe (a Capture probe) bind indirectly to a target nucleic acid which is obtained from a biological sample. Each of the two probes hybridizes to a target-specific oligonucleotide which in turn binds to the target nucleic acid obtained from a biological sample

FIG. 4: Shows protein target detection using the present invention. (A) Shows a Target- and Sample-Specific Probe and a first type Protein-targeting Probe. The first type protein probe includes a first region capable of binding to target protein (shown in black) and a second region including a partially double-stranded nucleic acid (shown in red and green). (B) Shows the target nucleic acid binding region of the Target- and Sample-Specific probe bound to a portion of the second region of the first type protein probe; here, the portion of second region of the first type protein probe is not cleaved from the first region of the protein probe. (C) and (D) Show the target nucleic acid binding region of the Target- and Sample-specific probe bound to a portion of the second region of the first type protein probe; in these, the portion of the second region of the protein probe is cleaved from the protein targeting region of the protein probe. (E) Shows Target- and Sample-Specific Probe and a second type Protein-targeting Probe including a single-stranded nucleic acid. The second type protein probe includes a first region capable of binding to target protein (shown in black) and a second region including a single-stranded nucleic acid (shown in green). (F) Shows the target nucleic acid binding region of the Target- and Sample-Specific probe bound to a portion of the second region of the second type protein probe; here, the portion of second region of the second type protein probe is not cleaved from the first region of the protein probe. (G) and (H) Show the target nucleic acid binding region of the Target- and Sample-specific probe bound to a portion of the second region of the second type protein probe; in these, the portion of the second region of the second type protein probe is cleaved from the protein targeting region of the protein probe.

FIG. 5: Shows a six-position probe backbone in which the first four positions (numbered 1 to 4) identify a target and the fifth and sixth positions identify a sample. A target-binding region is shown as a thick black line.

FIG. 6: Shows a four-position probe backbone in which two positions identify a target and two positions identify a sample.

FIG. 7: Shows a seven-position probe backbone in which four positions identify a target and three positions identify a sample.

FIG. 8: Shows a six-position probe backbone in which three positions identify a target and three positions identify a sample.

FIG. 9: Shows a probe backbone having only a single position; the single position identifying a sample.

FIG. 10: Shows a two-position probe backbone in which one position identifies a target and one position identifies a sample.

FIG. 11: Shows four examples of four-position target-identifying regions (the positions are numbered 1 to 4). Each configuration shown identifies a district target (i.e., Target 1 to Target 4).

FIG. 12: Shows examples of two position sample-identifying regions (the positions are numbered 5 and 6). Each column shows sample-identifying regions for one of the eight samples (Samples A to H).

FIG. 13: Shows four example six position probes, each identifying a specific target and a specific sample.

FIG. 14: Shows two exemplary sets of six-position probes used to target two different RNAs (i.e., ARL2 and ARMET). Here, the first four positions identify the target and the last two spots identify the sample.

FIG. 15: Shows data described in Example 1 in which levels of twenty-five target nucleic acids obtained from eight samples apiece were detected.

FIG. 16: Shows data described in Example 2 in which levels of twenty-six target nucleic acids obtained from thirty-two samples apiece were detected. Only data from three samples, i.e., samples B, D, and X, are shown.

FIG. 17 to FIG. 20: Show data described in Example 2 which compares results obtained when a single species of probe was hybridized (i.e., a single-plexed assay) with results obtained thirty two distinct probes were simultaneously hybridized (i.e., a multi-plexed assay). Data is shown for four exemplary samples.

### DETAILED DESCRIPTION OF THE INVENTION

The present invention is based in part on new probes, compositions, methods, and kits for simultaneously detecting nucleic acid or protein targets in a plurality of samples.

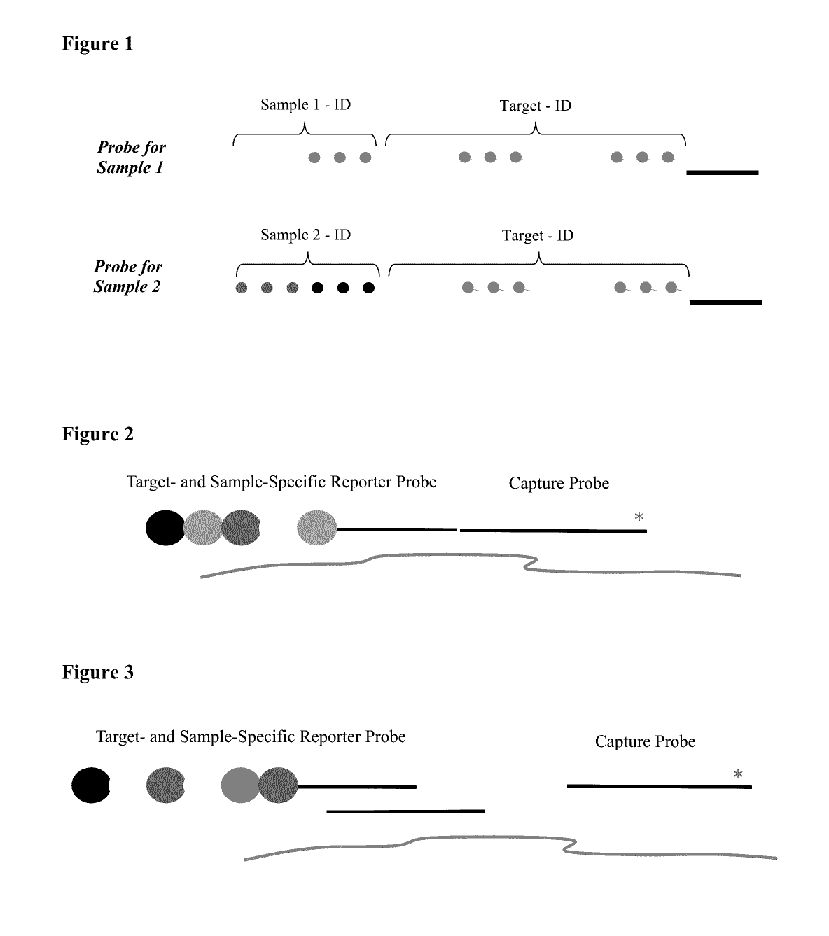

Unlike previously-described probes, the present invention relates to a probe having a backbone that includes at least one region capable of identifying a target nucleic acid or protein in a sample and at least one region capable of identifying the sample. Two exemplary probes are illustrated in FIG. 1. Each probe in the illustration includes three regions: a “Target-ID” region, a “Sample-ID” region, and a region that is capable of binding to a target nucleic acid. The region capable of binding to a target nucleic acid is shown here as a dark black line. As used herein, the term “Target-ID” region is synonymous with a region capable of binding to at least a plurality of labeled single-stranded oligonucleotides, in which the plurality of labeled single-stranded oligonucleotides identifies the target nucleic acid in the sample; this region is shown here as a dark gray line. As used herein, the term “Sample-ID” region is synonymous with a region capable of binding to a plurality of labeled single-stranded oligonucleotides, in which the plurality of labeled single-stranded oligonucleotides identifies the sample; this region is shown here as a light gray line.

The region capable of binding to a target nucleic acid is preferably at least 15 nucleotides in length, and more preferably is at least 20 nucleotides in length. In specific embodiments, the target-specific sequence is approximately 10 to 500, 20 to 400, 25, 30 to 300, 35, 40 to 200, or 50 to 100 nucleotides in length.

The probes illustrated in FIG. 1 have six positions with each position distinguishable by being hybridized to three oligonucleotides having the same color label. In both probes, the “Target-ID” region comprises four positions (hybridized to alternating blue- and yellow-labeled oligonucleotides). The “Sample ID” regions comprise two positions. The sample 1 probe's first two positions are hybridized to yellow- and blue-labeled oligonucleotides, respectively, and the sample 2 probe's first two positions are hybridized to green- and red-labeled oligonucleotides, respectively. The colors shown in FIG. 1, and elsewhere in this disclosure, are non-limiting; other colored labels and other detectable labels known in the art can be used in the probes of the present invention.

For a “Target-ID” region, the linear order of labels provides a signal identifying the target nucleic acid. For a “Sample-ID” region, the linear order of labels provides a signal identifying the sample.

Each labeled oligonucleotide may be labeled with one or more detectable label monomers. The label may be at a terminus of an oligonucleotide, at a point within an oligonucleotide, or a combinations thereof. Oligonucleotides may comprise nucleotides with amine-modifications, which allow coupling of a detectable label to the nucleotide.

Labeled oligonucleotides of the present invention can be labeled with any of a variety of label monomers, such as a fluorochrome, quantum dot, dye, enzyme, nanoparticle, chemiluminescent marker, biotin, or other monomer known in the art that can be detected directly (e.g., by light emission) or indirectly (e.g., by binding of a fluorescently-labeled antibody). Preferred examples of a label that can be utilized by the invention are fluorophores. Several fluorophores can be used as label monomers for labeling nucleotides including, but not limited to GFP-related proteins, cyanine dyes, fluorescein, rhodamine, ALEXA Flour™, Texas Red, FAM, JOE, TAMRA, and ROX. Several different fluorophores are known, and more continue to be produced, that span the entire spectrum.

Labels associated with each position (via hybridization of a position with a labeled oligonucleotide) are spatially-separable and spectrally-resolvable from the labels of a preceding position or a subsequent position.

Each position in a probe may be hybridized with at least one labeled oligonucleotide. Alternately, a position may be hybridized with at least one oligonucleotide lacking a detectable label. Each position can hybridize to 1, 2, 3, 4, 5, 6, 7, 8, 9, 10, 11, 12, 13, 14, 15, 16, 17, 18, 19, 20, or 21 to 100 labeled (or unlabeled) oligonucleotides or more. The number of labeled oligonucleotides hybridized to each position depends on the length of the position and the size of the oligonucleotides. A position may be between about 300 to about 1500 nucleotides in length. The length of the labeled oligonucleotides may vary from about 20 to about 55 nucleotides in length. The oligonucleotides are designed to have melting/hybridization temperatures of between about 65 and about 85° C., e.g., about 80° C. For example, a position of about 1100 nucleotides in length may hybridize to between about 25 and about 45 oligonucleotides, each oligonucleotide about 45 to about 25 nucleotides in length. In embodiments, each position is hybridized to about 34 labeled oligonucleotides of about 33 nucleotides in length. The labeled oligonucleotides are preferably single-stranded DNA. Exemplary oligonucleotides are listed in Table 1.

The number of target nucleic acids and samples detectable by a set of probes depends on the number of positions that the probes' backbones include.

The number of positions on a probe's backbone ranges from 1 to 50. In yet other embodiments, the number of positions ranges from 1, 2, 3, 4, 5, 6, 7, 8, 9, 10 to 15, 20, 30, 40, or 50, or any range in between. Indeed, the number of positions (for detecting a target nucleic acid and/or for detecting a sample) on a backbone is without limit since engineering such a backbone is well-within the ability of a skilled artisan.

A probe may be chemically synthesized or may be produced biologically using a vector into which a nucleic acid encoding the probe has been cloned.

The labeled oligonucleotides hybridize to their positions under a standard hybridization reaction, e.g., 65° C., 5×SSPE; this allows for self-assembling reporter probes. Probes using longer RNA molecules as labeled oligonucleotide (e.g., as described in US2003/0013091) must be pre-assembled at a manufacturing site rather than by an end user and at higher temperatures to avoid cross-linking of multiple backbones via the longer RNA molecules; the pre-assembly steps are followed by purification to remove excess un-hybridized RNA molecules, which increase background. Use of the short single-stranded labeled oligonucleotide greatly simplifies the manufacturing of the probes and reduces the costs associated with their manufacture.

The probes of the present invention can be used to directly hybridize to a target nucleic acid obtained from a biological sample. FIG. 2 illustrates a composition of including probes of this embodiment. Such a composition includes at least two single-stranded nucleic acid probes: a target- and sample-specific reporter probe and a capture probe. As used herein, a target- and sample-specific reporter probe is synonymous with a single-stranded nucleic acid probe comprising at least three regions as described in the above-mentioned aspects of the invention. The capture probe comprises at least one affinity reagent which is shown as an asterisk. As used herein, the capture probe is synonymous with a second single-stranded nucleic acid probe comprising at least two regions as described in the above-mentioned aspects of the invention. Each of the six positions in the illustrated target- and sample-specific reporter probe is identified by a colored circle. The target nucleic acid obtained from a biological sample is shown as a blue curvilinear line. Probes capable of directly hybridizing to a target nucleic acid obtained from a biological sample and capable of identifying the target nucleic acid (but incapable of identifying a sample) have been described in, e.g., US2003/0013091, US2007/0166708, US2010/0047924, US2010/0112710, US2010/0261026, US2010/0262374, US2011/0003715, US2011/0201515, US2011/0207623, US2011/0229888, US2013/0230851, US2014/0005067, US2014/0162251, US2014/0371088, and US2016/0042120, each of which is incorporated herein by reference in its entirety.

The aforementioned US Patent Publications further describe immobilizing, orientating, and extending a probe pair hybridized to a target nucleic acid.

The at least one affinity moiety may be attached to the capture probe by covalent or non-covalent means. Various affinity moieties appropriate for purification and/or for immobilization are known in the art. Preferably, the affinity moiety is biotin, avidin, or streptavidin. Other affinity tags are recognized by specific binding partners and thus facilitate isolation and immobilization by affinity binding to the binding partner, which can be immobilized onto a solid support. A target- and sample-specific reporter probe may also comprise at least one affinity moiety, as described above.

The probes of the present invention can be used to indirectly hybridize to a target nucleic acid obtained from a biological sample. FIG. 3 illustrates a composition including probes of this embodiment. Such a composition includes at least two single-stranded nucleic acid probes: a target- and sample-specific reporter probe and a capture probe. Additionally, the composition includes two oligonucleotides that are capable of directly hybridizing to a target nucleic acid obtained from a biological sample, i.e., target-specific oligonucleotides. In FIG. 3, the target- and sample-specific reporter probe hybridizes to a target-specific oligonucleotide (shown in red) which hybridizes to the target nucleic acid obtained from a biological sample (shown as a blue curvilinear line); the capture probe hybridizes to another target-specific oligonucleotide (shown in green) which hybridizes to the target nucleic acid obtained from a biological sample. Probes capable of indirectly hybridizing to a target nucleic acid obtained from a biological sample and capable of identifying the target nucleic acid (but incapable of identifying a sample) have been described in, e.g., US2014/0371088, which is incorporated herein by reference in its entirety.

In the hybridization/detection system, a probe's target binding region hybridizes to a region of a target-specific oligonucleotide. Thus, the probe's target binding region is independent of the ultimate target nucleic acid obtained from a sample. This allows economical and rapid flexibility in an assay design, as the target (obtained from a biological sample)-specific components of the assay are included in inexpensive and widely-available DNA oligonucleotides rather than the more expensive probes. Therefore, a single set of indirectly-binding probes can be used to detect an infinite variety of target nucleic acids in different experiments simply by replacing the target-specific oligonucleotide portion of the assay.

The aforementioned US Patent Publication further describes immobilizing, orientating, and extending a probe pair hybridized to target-specific oligonucleotides that are in turn hybridized to a target nucleic acid obtained from a biological sample.

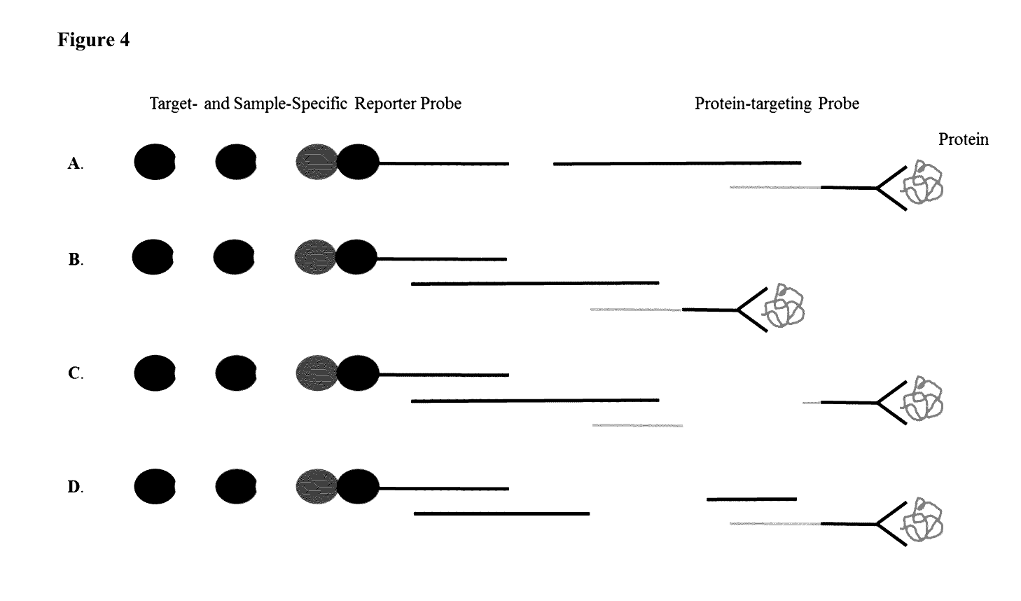

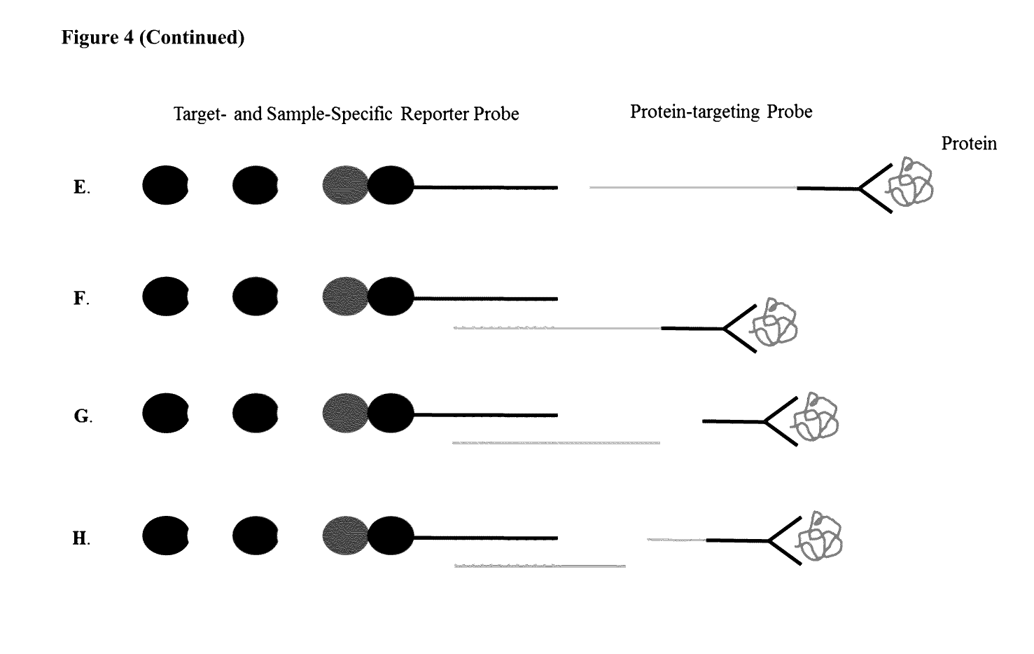

The single-stranded nucleic acid probes of the present invention can be used for detecting a target protein obtained from a biological sample. FIG. 4 illustrates this aspect, which includes at least one single-stranded nucleic acid probe having a Target- and Sample-Specific Reporter region and at least one protein probe having a first region capable of binding to a target protein and a second region including a partially double-stranded nucleic acid (A to D) or including a single-stranded nucleic acid (E to H). The region capable of binding to a target protein includes an antibody, a peptide, an aptamer, or a peptoid. The antibody can be obtained from a variety of sources, including but not limited to polyclonal antibody, monoclonal antibody, monospecific antibody, recombinantly expressed antibody, humanized antibody, plantibodies, and the like. The target binding region of the Target- and Sample-Specific Reporter Probe binds to a portion of the second region of the protein probe. A capture probe, as illustrated in FIGS. 2 and 3 may be included (not shown). The second region of the protein probe can include a linker, e.g., a photocleavable linker, which when cleaved, can release a portion of the second region from the first region. The linker may be 5′ to the double-stranded portion or may be 3′ to the double-stranded portion or the linker may be 5′ to the single-stranded nucleic acid, within the single-stranded nucleic acid, or 3′ to the single-stranded nucleic acid. Alternately, the second region of the protein probe can be released from the first region by other methods known in the art (e.g., by denaturing the double-stranded portion and by digestion). The target protein obtained from a biological sample is identified in FIG. 4 as “Protein”. Probes and methods for binding a target protein obtained from a biological sample and identifying the target protein (but incapable of identifying a sample) have been described, e.g., in US2011/0086774 and US2016/0003809, the contents of which are incorporated herein by reference in their entireties.

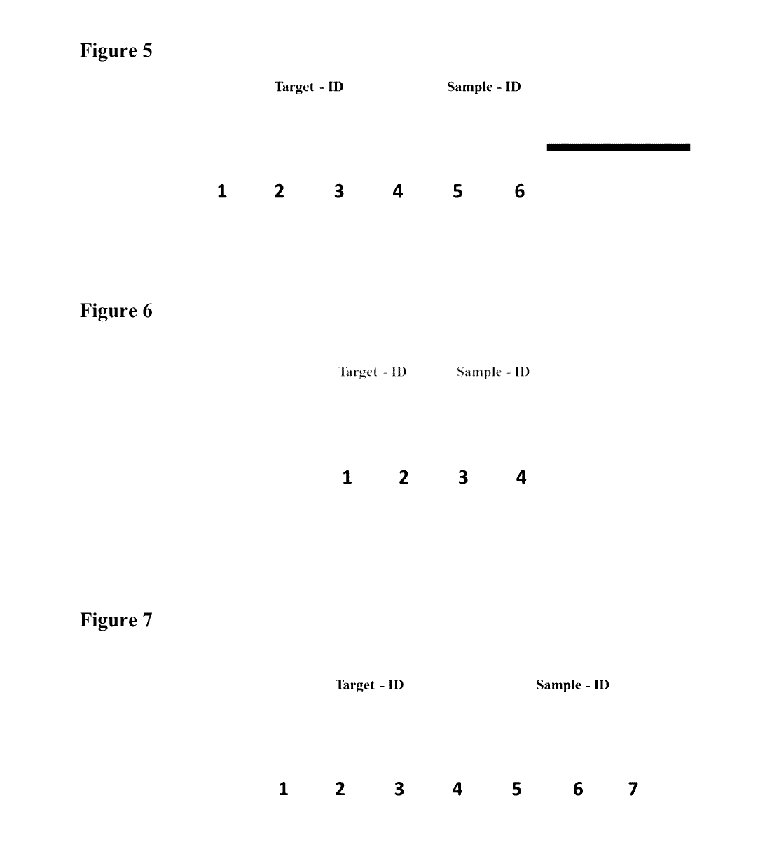

A probe's backbone is preferably single-stranded DNA, RNA or PNA. It may include one or more positions, e.g., two or more, three or more, four or more, five or more, six or more, seven or more, eight or more, nine or more, ten or more, eleven or more, twelve or more, and twenty or more positions, each capable of binding to at least a plurality of single-stranded oligonucleotides, e.g., labeled oligonucleotides. There is no upper limit to the number of positions that a probe backbone may contain, e.g., twenty or more, fifty or more, and one hundred or more positions. As described above, the backbone may include, at least, a region for binding to a target nucleic acid, a region for identifying a target, and a region for identifying a sample. The backbone shown in FIG. 5 has six positions with four positions for identifying a target and two positions for identifying a sample; the region for binding to a target nucleic acid is shown here as black line. A backbone may have a fewer number of positions (e.g., five, four, three, two, and one; see, e.g., FIGS. 6, 9 and 10) or a greater number of positions (e.g., seven, eight, nine, ten, or more; see, e.g., FIG. 7). Any number of positions for a second region can be combined with any number of positions for a third region. More specifically, the probe may include a second region having one, two, three, four, five, six, seven, eight, nine, ten, or more positions and a third region having one, two, three, four, five, six, seven, eight, nine, ten, or more positions. The region for identifying a target nucleic acid may be located distally to the target binding domain (as shown FIG. 5) or the region for identifying a target nucleic acid may be located adjacent to the target binding domain (as shown FIGS. 1 and 10). The number of regions for identifying a target nucleic acid may be the same as the number of regions for identifying a sample (as shown in FIGS. 6, 8, and 10) or the number of regions for identifying a target nucleic acid may be greater than the number of regions for identifying a sample (as shown in FIGS. 1, 5, and 7).

In embodying probes, a first plurality of labeled single-stranded oligonucleotides is not identical to a second plurality of labeled single-stranded oligonucleotides.

In embodiments, a single-stranded oligonucleotide may lack a label monomer. Thus, a position of a second region and/or a third position may be hybridized with at least one oligonucleotide lacking a detectable label. In these embodiments, a probe will have a “dark spot” adjacent to a position having a detectable signal. The term “dark spot” refers to a lack of signal from a label attachment site on a probe. Dark spots add more coding permutations and generate greater reporter diversity in a population of probes.

In embodiments, at least one probe may comprise a single-stranded or double-stranded RNA, DNA, PNA, or other polynucleotide analogue spacer between its first region and its second region and/or between its first region and its third region. The spacer may be double-stranded DNA. The spacer may have similar mechanical properties as the probe's backbone. The spacer may be of any length, e.g., 1 to 100 nucleotides, e.g., 2 to 50 nucleotides. The spacer can be shorter than 20 nucleotides or longer than 40 nucleotides and it can be 20 to 40 nucleotides long. Non-limiting examples of a spacer includes the sequences covered by SEQ ID NO: 809 to SEQ ID NO: 813.

A probe backbone may include only a single position, with the single position identifying the sample (as shown in FIG. 9).

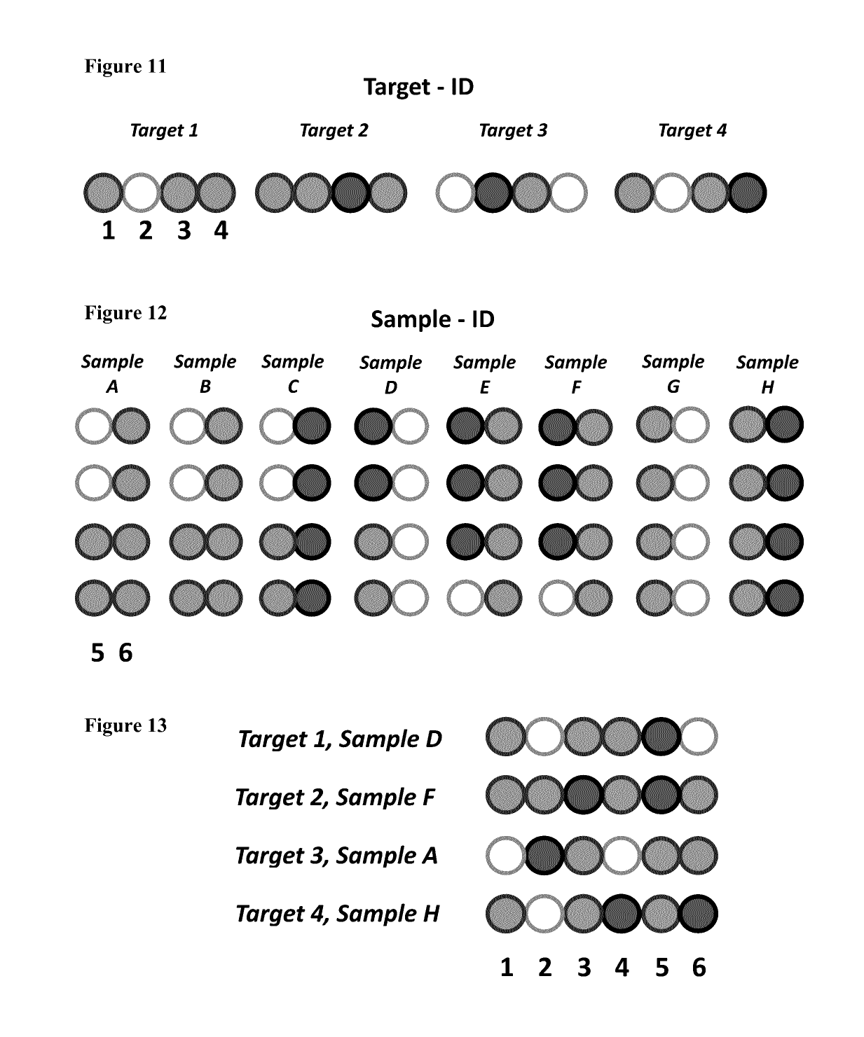

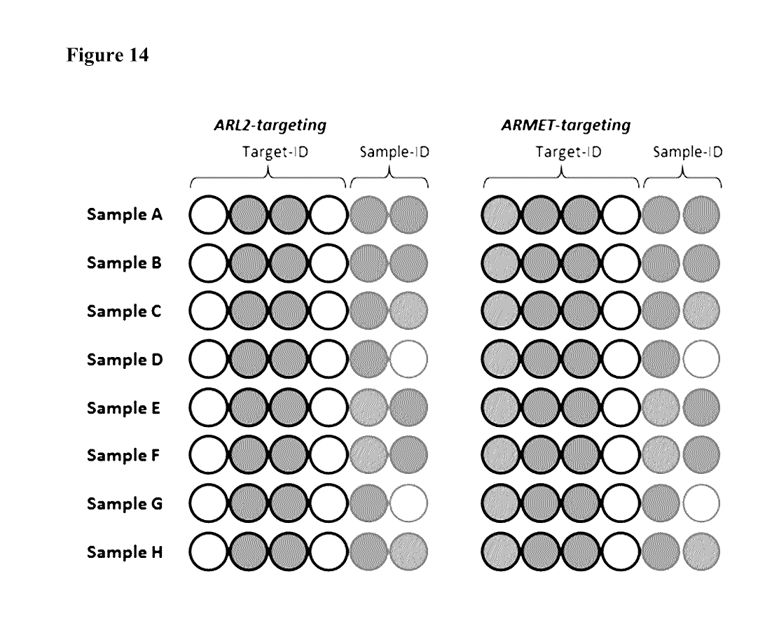

FIGS. 11 to 14 illustrate formation of six-position probes. FIG. 11 shows four examples of four-position target-identifying regions (the positions are numbered 1 to 4). Each example is capable of identifying a district target nucleic acid (identified as Target 1 to Target 4). FIG. 12 shows examples of two position sample-identifying regions (the positions are numbered 5 and 6). Each column shows sample-identifying regions for one of the eight samples (Samples A to H). Each row represents the eight sample-identifying positions for each of the target-identifying regions shown in FIG. 11, such that the top row corresponds to the “Target 1” target-identifying region of FIG. 11 and the bottom row corresponds to the “Target 4” target-identifying region. FIG. 13 shows four exemplary six-position probes that are constructed when the four-position target-identifying regions (of FIG. 11) are combined with the two position sample-identifying regions (of FIG. 12). Each six-position probe is capable of identifying a specific target and a specific sample. FIG. 14 shows two sets of six-position probes used in Example 1. The left probe set was used when ARL2 was the target nucleic acid obtained from a sample; the right probe set was used when ARMET was the target nucleic acid obtained from a sample. As in FIGS. 1, 5, and 13, four positions identify the target and two positions identify the sample.

Probes can be detected and quantified using commercially-available cartridges, software, systems, e.g., the nCounter® System using the nCounter® Cartridge.

For the herein-described probes, association of label code to target sequence is not fixed. This allows a single set of backbones to be used to generate different codes during hybridization to different samples, by combining it with differently colored pools of oligonucleotides. Following hybridization, the samples are pooled and processed together, as the resulting barcodes will be unique to each sample and can be assigned back to their sample of origin following data collection. An example is the following:

A set of 96 six-position backbones may be used to detect up to 96 different target nucleic acids (either directly or indirectly) or proteins. Oligonucleotide pools (i.e., a plurality of labeled single-stranded oligonucleotides) for positions 1 to 4 of each backbone are associated with fixed colors, such that the four position code for a particular target nucleic acid/protein is always the same, regardless of the hybridization reaction. Positions 5 and 6, although they have a fixed sequence for any given backbone, are given a different color for each sample by coupling the oligonucleotide pool for each position separately to different colored-labels. By producing a differentially-labeled probe for each sample, samples (comprising the target nucleic acid and hybridized probes) can be pooled after the hybridization reaction. The pooled samples can then be processed together and all labeled probes (i.e., barcodes) are imaged together. Then, obtained data is de-convoluted back into the original samples after scanning, thereby tallying the identity of all the barcodes in the image. Such multiplexing greatly increases the throughput of the system.

In a six-position, four color system (i.e., yellow, red, blue, and green fluorophores), the possible combinations of gene-plex and sample-plex are many, depending on how many positions are dedicated to identifying a target nucleic acid or protein and how many positions are dedicated to identifying a sample. When plexing eight samples together (two positions of a probe dedicated for sample identity), each column of a 96-well plate is pooled and each pool is detected on a single lane of a twelve lane cartridge, e.g., an nCounter® Cartridge. When plexing thirty-two samples together (three positions of a probe dedicated for sample identity), a 384-well plate can be detected on a single twelve lane cartridge, e.g., an nCounter® Cartridge.

A kit including six-position probes contains reagents and probes sufficient to detect up to 96 target nucleic acids or proteins in a 96 well format or up to 24 target nucleic acids or proteins in a 384 well format.

An exemplary protocol, using NanoString Technologies®'s nCounter® systems for detecting nucleic acids, is described as follows. Approximately 50 to 100 ng of total RNA per sample and/or a lysate of about 1,000 to about 2500 cells per sample in a total volume of about 5 μl (volume adjusted with RNAse free water, if necessary). Samples are added to a thermocycler-compatible 96-well plate. For a 96-well plate of samples, a kit may include eight tubes (labeled A to H) of TagSet reagents, with each tube containing enough reagents to set up one row of assays (12 samples). A mastermix is made for each of tubes A to H (i.e., Mastermix A to Mastermix H) by adding hybridization buffer and the target-specific first and second probes diluted to the appropriate concentration. 10 μl of Mastermix A is pipetted into each well in row A, 10 μl of Mastermix B is pipetted into each well in row B, and so forth, until Mastermix H has been pipetted. The plate is sealed and heated overnight at about 67° C. in a thermocycler with a heated lid, allowing hybridization of labeled oligonucleotides to appropriate positions of a probe and allowing the probes to hybridize to their target nucleic acids. If the probe indirectly binds to a target nucleic acid obtained from a sample, additional target-specific oligonucleotides (which are bound by a probe and bind to the target nucleic acid obtained from a sample) are include in mastermixes. These target-specific oligonucleotides may not be included in kit as they can be commercially synthesized. The sealing is removed from the plate and assays (samples) for each column are pooled into a twelve-tube strip such that a first pooled sample will contain samples from wells A1 to H1, a second pooled sample will contain samples from wells A2 to H2, and so forth. The twelve-tube strip is placed into a NanoString Technologies® Prep Station and processed using a standard nCounter® protocol, which ultimately scans an nCounter® cartridge and de-convolutes data into individual samples by the ordering of labeled oligonucleotides hybridized to the probes.

A target nucleic acid obtained from a sample may be DNA or RNA and preferably messenger RNA (mRNA).

Probes of the present invention can be used to detect a target nucleic acid or protein obtained from any biological sample. As will be appreciated by those in the art, the sample may comprise any number of things, including, but not limited to: cells (including both primary cells and cultured cell lines), cell lysates or extracts (including but not limited to protein extracts, RNA extracts; purified mRNA), tissues and tissue extracts (including but not limited to protein extracts, RNA extracts; purified mRNA); bodily fluids (including, but not limited to, blood, urine, serum, lymph, bile, cerebrospinal fluid, interstitial fluid, aqueous or vitreous humor, colostrum, sputum, amniotic fluid, saliva, anal and vaginal secretions, perspiration and semen, a transudate, an exudate (e.g., fluid obtained from an abscess or any other site of infection or inflammation) or fluid obtained from a joint (e.g., a normal joint or a joint affected by disease such as rheumatoid arthritis, osteoarthritis, gout or septic arthritis) of virtually any organism, with mammalian samples being preferred and human samples being particularly preferred; environmental samples (including, but not limited to, air, agricultural, water and soil samples); biological warfare agent samples; research samples including extracellular fluids, extracellular supernatants from cell cultures, inclusion bodies in bacteria, cellular compartments, cellular periplasm, and mitochondria compartment.

A probe's region capable of binding to a target protein include molecules or assemblies that are designed to bind with at least one target protein, at least one target protein surrogate, or both; and can, under appropriate conditions, form a molecular complex comprising the protein probe and the target protein. The terms “protein”, “polypeptide”, “peptide”, and “amino acid sequence” are used interchangeably herein to refer to polymers of amino acids of any length. The polymer may be linear or branched, it may comprise modified amino acids, and it may be interrupted by non-amino acids or synthetic amino acids. The terms also encompass an amino acid polymer that has been modified, for example, by disulfide bond formation, glycosylation, lipidation, acetylation, phosphorylation, or any other manipulation, such as conjugation with a labeling component. As used herein the term “amino acid” refers to either natural and/or unnatural or synthetic amino acids, including but not limited to glycine and both the D or L optical isomers, and amino acid analogs and peptidomimetics.

The biological samples may be indirectly derived from biological specimens. For example, where the target nucleic acid is a cellular transcript, e.g., an mRNA, the biological sample of the invention can be a sample containing cDNA produced by a reverse transcription of mRNA. In another example, the biological sample of the invention is generated by subjecting a biological specimen to fractionation, e.g., size fractionation or membrane fractionation.

The biological samples of the invention may be either “native,” i.e., not subject to manipulation or treatment, or “treated,” which can include any number of treatments, including exposure to candidate agents including drugs, genetic engineering (e.g., the addition or deletion of a gene).

In embodiments, a first sample differs from a second sample in an experimental manipulation, e.g., the presence of absence of an applied drug or concentration thereof. This embodiment is particularly significant in cultured cells which may be exposed to a variety of controlled conditions.

In some embodiments, the probes, compositions, methods, and kits described herein are used in the diagnosis of a condition. As used herein the term “diagnose” or “diagnosis” of a condition includes predicting or diagnosing the condition, determining predisposition to the condition, monitoring treatment of the condition, diagnosing a therapeutic response of the disease, and prognosis of the condition, condition progression, and response to particular treatment of the condition. For example, a blood sample can be assayed according to any of the probes, methods, or kits described herein to determine the presence and/or quantity of markers of a disease or malignant cell type in the sample (relative to the non-diseased condition), thereby diagnosing or staging the a disease or a cancer.

A kit of the present invention can include other reagents as well, for example, buffers for performing hybridization reactions, linkers, restriction endonucleases, and DNA ligases. A kit also will include instructions for using the components of the kit, including, but not limited to, information necessary to hybridize labeled oligonucleotides to a probe, to hybridize a probe to a target-specific oligonucleotide, and/or to hybridize a probe or target-specific oligonucleotide to a target nucleic.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless defined otherwise, all technical and scientific terms used herein have the same meaning as commonly understood by one of ordinary skill in the art to which the invention pertains. Although other probes, compositions, methods, and kits similar, or equivalent, to those described herein can be used in the practice of the present invention, the preferred materials and methods are described herein. It is to be understood that the terminology used herein is for the purpose of describing particular embodiments only, and is not intended to be limiting.

### EXAMPLES

The following examples are put forth so as to provide those of ordinary skill in the art with a complete disclosure and description of how to practice the present invention, and are not intended to limit the scope of what the inventors regard as the invention. Efforts have been made to ensure accuracy with respect to numbers used (e.g., amounts and concentrations) but some experimental errors and deviations should be accounted for. Unless indicated otherwise, temperature is in degrees Centigrade and pressure is at or near atmospheric.

### Example 1

### Eight Sample-Plex Assay Using Probes Comprising Four Positions for Target Identification and Two Positions for Sample Identification

This Example provides data using probes have six positions which include four positions for target identification and two positions for sample identification. Such probes can be detected with the NanoString Technologies® Digital Analyzer post sample processing.

Single-stranded nucleic acid probes used in this assay included a first region of a unique thirty-five deoxynucleotide target binding domain and six consecutive positions for binding labeled oligonucleotides. Each position was 1100 deoxynucleotides in length and had a unique sequence. The first four positions, which were adjacent to the target binding domain, were for identifying the target nucleic acid and the next two positions were for identifying the sample.

Each position of a probe backbone was an approximately 1100 nucleotide sequence. Twenty-four approximate 1100-nucleotide sequences, as described in US2010/0047924 (the contents of which are incorporated herein by reference in its entirety) were used to form backbones. For each position, a set of single-stranded DNA oligonucleotides was designed; together these oligonucleotides were complementary to the entirety of each 1100-nucleotide sequence. Each individual oligonucleotide in the set was designed to have melting temperature (Tm) of approximately 80° C. in 5×SSPE (typically ranging from 78 to 85° C.). Sequences for the single-stranded DNA oligonucleotides are listed in Table 1. All oligonucleotides were synthesized with 5′ amine modifications to attach fluorescent labels. Fluorescent labels coupled to these 5′ amine modifications were Alexa Fluor 488 5-TFP (2,3,5,6-Tetrafluorophenyl Ester) (“Blue”), Alexa Fluor 546 NHS Ester (Succinimidyl Ester) (“Green”), Texas Red-X NHS Ester (Succinimidyl Ester) (“Yellow”), or Alexa Fluor 647 NHS Ester (Succinimidyl Ester) (“Red”) Coupling used standard methods.

Hybridization reactions were performed as described in, e.g., US2014/0371088.

This Example is illustrated in FIG. 3. Here, six-position Target- and Sample-Specific Reporter probes (hereinafter “Backbones”) each having a thirty-five deoxynucleotide target binding domain forms a complex with a target-specific oligonucleotide. The target-specific oligonucleotide is complementary to the thirty-five deoxynucleotide target binding domain and is complementary to target nucleic acid obtained from a sample. The target-specific oligonucleotide is shown in red in FIG. 3 (hereinafter “Oligo A”). A Capture Probe (hereinafter “UCP-3BF2”) includes a twenty-five deoxynucleotide target binding region and region comprising at least one affinity moiety, e.g., biotin. The capture probe binds to a second target-specific oligonucleotide shown in green in FIG. 3 (hereinafter “Oligo B”). Oligo B has a region complementary to UCP-3BF2 and a region complementary to the target nucleic acid obtained from a sample.

In 30 μl hybridization reactions, the following reagents were combined (to a final concentration shown): SSPE (5x), Oligo A's (20 pM each), Oligo B's (100 pM each), UCP-3BF2 (5200 pM), 26 Backbones (25 pM each), labeled oligonucleotides (at a 2:1 ratio relative to backbone sequence), and cell lysate from A431 cells (endogenous RNAs from these lysates are the target nucleic acid obtained from a sample). Hybridization reactions were performed in separate PCR tubes in a thermocycler overnight at 67° C. Backbone sequences and labeled oligos used for each sample are listed in Table 2 and Table 3.

After the hybridization reactions, samples were either processed as single samples (a single-plex assay) or pooled into a combined sample with seven other samples (a multi-plex assay). Samples were processed on a NanoString Technologies® Prep Station and codes were counted with a Gen2 Digital Analyzer.

FIG. 14 shows two exemplary sets of six position probes used in this Example.

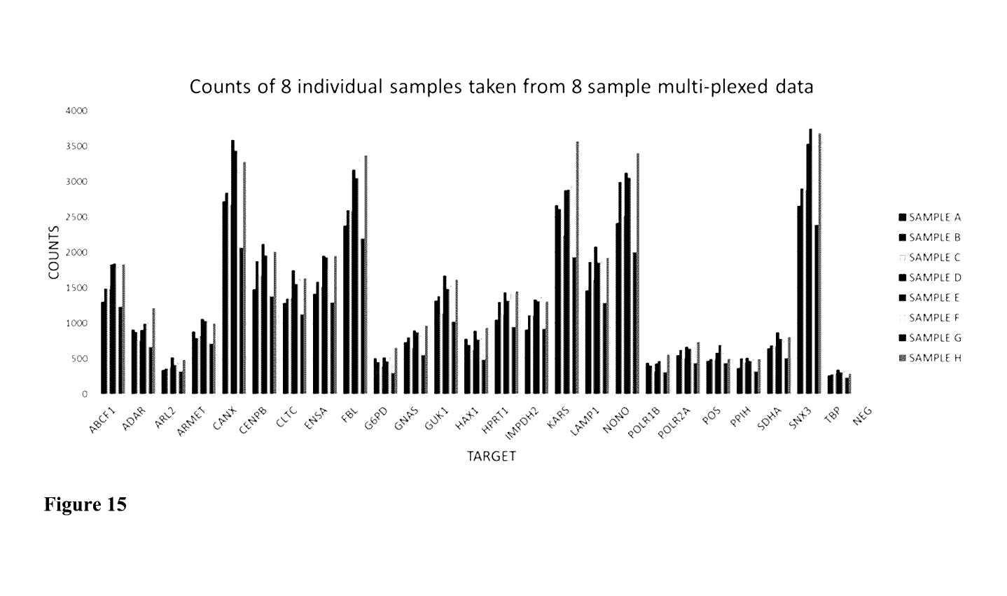

FIG. 15 shows the counts for all targets in all samples detected in this Example. Here, eight independent hybridization reactions with differing amounts of target nucleic acid and different mixes of labeled oligonucleotides to identify samples were used (see Table 3 for oligonucleotide colors used for each sample). Each reaction contained probes against twenty-five nucleic acid targets and one negative control (“NEG”) which lacked a target nucleic acid in the hybridization. 15 μl of each hybridization reaction was pooled (120 μl total) and 30 μl of this combined sample was loaded onto a lane on a NanoString Technologies® Prep Station. Counts were determined with a NanoString Technologies® Digital Analyzer. Counts are shown in FIG. 15.

TABLE 1
Oligonucleotide sequences

| | | 1100 bp |
|---|---|---|
| SEQ | | nucleotide |
| ID | | sequence |
| NO: | SEQUENCE | number |
| 1 | AGGTAGACAAAAGTAAGCCAGTGGCACAGTGAGGA | 1 |
| 2 | AGATGAGCGAGCTGAGGACAATGACGG | 1 |
| 3 | AGTCGGAGGAATCAGAGCGGTGAGACA | 1 |
| 4 | AGTGGAGGATATCAAAGATAAGAGCATAGGGAAATGCA | 1 |
| 5 | ACAATGGAAACGTCCCAAGGTGGAAGCG | 1 |
| 6 | TGGGAGAATGAAGAGGTAAGCAAATAGAAGACGTAGGGA | 1 |
| 7 | ACATGAAACCATGCAGAAGATAAGAAAATGCCAGAA | 1 |
| 8 | TACGACGGTGAGAGAAATCAACCAGTACAAGCGCTGA | 1 |
| 9 | ACAGCTACCGAGGTAGCGAGATGAACAAGA | 1 |
| 10 | TGCGAACCTCAGGAACTCAAGAAGTAGCGAA | 1 |
| 11 | ATCGACCGGGTCGGGAAAGTCGAGAA | 1 |
| 12 | ATAAGAACGTACCAGGGATACAGAACTAGGGACGT | 1 |
| 13 | AGGAGGGTGGGACGATACGGCGCTGAA | 1 |
| 14 | ACGGGTGGGAGGGTAACAGGGTGGAA | 1 |
| 15 | AAGTAAGAGACTAAGGAACTGAAACAGCTAACAGGCT | 1 |
| 16 | AAGGGAACATGGAGAAATAAAGACACTGGAGCGCA | 1 |
| 17 | TCCGGAAGATAGAGAAAATGAGAGCGTGAAACCA | 1 |
| 18 | TGAAAGGGATCAAGAGGTGACGGAGCATAGA | 1 |
| 19 | AAGCTGAAACAAATAGGGAAGCTGAAGACCA | 1 |
| 20 | TAAGCGGGCTGCCAAAGATAAGAGAGTGACA | 1 |
| 21 | AGATACGCGCCGTGGAGAAGTGCAGGACA | 1 |
| 22 | TAAAACAATGGCCGCATCAGGCCGGG | 1 |
| 23 | TGAGGGCAATACAAGAGCTAGAAGAGTACCGCGA | 1 |
| 24 | TAGGAAGGTGGCACCAGTAAGGAAATAAGCCCA | 1 |
| 25 | TGAGGACATACACGAGTCGAAAAATAAGCGAGTCA | 1 |
| 26 | AACGCTAGGCCAACTGGCGGCATGGG | 1 |
| 27 | ACGGTGCGCGGGTCGACAGAGGTGT | 1 |
| 28 | ACAAGTGACAGGATGAAAGCATAAGAAGGTGACGCA | 1 |
| 29 | ACTAGGGCCATACAAAGAGTGGACCAATCCA | 1 |
| 30 | AACCTGCGAAGATAGGAGGATAACACCGGT | 1 |
| 31 | AGGGCAACTACAAGGATCAAAGGATGAAAGAATAAA | 1 |
| 32 | ACACTAAGGGCGTCCAACAGTACCGAAGTC | 1 |
| 33 | AGGGCGTCACAGGCTGAACAGAACTCAACCGAAGTCTAGA | 1 |
| 34 | AGAAATAACAGGAATAGCACAAGTAGGAACATAAACAGA | 2 |
| 35 | TGCACACCAATAAAGAGATACGGAAATAAAGACATAGAGACA | 2 |
| 36 | TCCGCAAATAAAGGGATGAAACAATAACCGGGTCGCGA | 2 |
| 37 | AGCTAACACCCATCGCCAGCTCGGGCA | 2 |
| 38 | AACTGATATCCTCGAAGGACTAGCAAGATGGAAACA | 2 |
| 39 | TGGACAAGTAAAAGGGTGAAGAAGTACCACAAACTCA | 2 |
| 40 | ACAGCTAAGACAAGTCGGGAAGTAAAAAAATCCA | 2 |
| 41 | AGGGTAACAGAATGGCGAAGTGAGCGAA | 2 |
| 42 | AGTAGAGCAATGACGGCATGGAGAAATGGGAA | 2 |
| 43 | AAAATCACGAGATAGACGAGTGGGCAGGT | 2 |
| 44 | AGGCGGGTGACCGGAATACGACAA | 2 |
| 45 | TGAGAGACTGCCGCAGTGCGAAAGTGG | 2 |
| 46 | CGGGATAAAAAAGCTGAAGGGAGTCAAACCA | 2 |
| 47 | TCAGCGGGATACAGAAGTAGAACAATGCACAGA | 2 |
| 48 | TGCGCAGGTAAGCGGGTGCAAGACT | 2 |
| 49 | AGAAAAGCTCGGCAAATCGCCGAATAGACAAA | 2 |
| 50 | TCGGAAGGTGAGCGGGTACGAAGAC | 2 |
| 51 | TGGCGGCCATAACGCCATGAGGGCAT | 2 |
| 52 | AGCAAAAGTACCCAAATAAGGCAGTCAGAGAGTG | 2 |
| 53 | ACGGGCTACAGCGGCTAGGCCGACT | 2 |
| 54 | ACAGCAGTGCGGGAAGTGAAAAAAGCTCGA | 2 |
| 55 | AGGATGCAACCATGAGCCAATAAAGGCGTCG | 2 |
| 56 | AGAGCTGCCAAGATGCGAAAGTGAGGACAT | 2 |
| 57 | AGACGAATAGAAAAATGAGACGATAGCGAGGTGACACCA | 2 |
| 58 | TACCAGGGTCCGGCAGGTAGCCAGA | 2 |
| 59 | TAGGGAAATAGAACGATCAAAGGATAGCGAAACGTCAGA | 2 |
| 60 | AACTCGGGAGCTAAAAGGATAACGGAATCAGATGT | 2 |
| 61 | ACAACGCAGATCAGAACCTGGGAGCA | 2 |
| 62 | AGTGGAGCCAATCAACAAGATAAGAAAAATGAAGGCA | 2 |
| 63 | TGGAAACGGTCCGGGCAGTGGA | 2 |
| 64 | ACGATCCAAGAGTAGGAAAATCAGAAGCGTACGA | 2 |
| 65 | AACTACAAGCGTCAGGGAGTAGAAAAGTAACACAATGAAGA | 2 |
| 66 | AACTAAGCACGTGAAAGAGTAGAGGAACTAGGAAACATCTAGA | 2 |
| 67 | AGAGGGCGTAACAAAGGTCGAAAACTCAAACACTACAAAAATGGGACA | 3 |
| 68 | AATAGCGCCCATCGGAAGCCTGAGCGGA | 3 |
| 69 | TAGCGGACTACGGCAGTAAAGAGATAGCGGAGC | 3 |
| 70 | TGAAAGCCTCAGGACGTGAACAAATGGGA | 3 |
| 71 | AGGATAACCGGGTAGGAGGGTGAAACAGA | 3 |
| 72 | TGAGACGGTAAGAAAAGTAAGAGAAGGTGCGAT | 3 |
| 73 | ATCGCGGGCGTCACGCAACGTGCAA | 3 |
| 74 | AAATGACGGAATAAAAGAATCGAGGAGGTCAAGGCGA | 3 |
| 75 | TAAGCGCGTGAGAGGATAGAAAGATCGAGCCA | 3 |
| 76 | TAGCGGGCCATCAGCGGCGTGCG | 3 |
| 77 | AGGAGTCGCACCACTCAAGAGCTAACCCGA | 3 |
| 78 | TCAGCGAGTACAGCGGGTAGAAGCGT | 3 |
| 79 | CGCGGGATAGAGGAAGTCCAAAGATCCCGA | 3 |
| 80 | ACTGCCAGCGTAGGAACACTGACCACAT | 3 |
| 81 | AGCACCATCAAAAGCTGAACGAGATGAGACACT | 3 |
| 82 | ACGCAGGATGAGAACGTCGCAAGCATGA | 3 |
| 83 | ACCGGGTGCAGAGCTAGGAGAATACCGCCGA | 3 |
| 84 | TCAGGCCACGTAGGAAGATCCAAGCC | 3 |
| 85 | TGGCACAGAGTCAAGACGCTAGAAAAATGAAGA | 3 |
| 86 | AGTCAGCAAAGTAGGGAGGGTGGGAGCA | 3 |
| 87 | TGAACGAGTGAGGACATAGGACGATCCCA | 3 |
| 88 | AAGTGACGGAATGACCAGGTGAGAAAGTAGGGCA | 3 |
| 89 | AATCAAGCAGTCAAAGCGTGAAGAAAACTAGAAGGCGT | 3 |
| 90 | AAAAGAGTGGAGAAGCTCCGACAGATACAAAAGT | 3 |
| 91 | AGAGGCCTGAGAGGGTCGGGCAAA | 3 |
| 92 | ATCCCAGACTCGGAGAATCGCGACAATGCA | 3 |
| 93 | AACGTGGCGCGGTGGGCGAGGTGCCGA | 3 |
| 94 | AATCACGCGAATGGACGGATATGTACACTGAAA | 3 |
| 95 | AAGCTCACAAAATAAGCGGATCGGGACA | 3 |
| 96 | TCGGAACCTGAGACGAATACAGACGATAAAGCAAT | 3 |
| 97 | AACCGACTAGACGAGTGCCAGGGT | 3 |
| 98 | AGGAACGTACCAAAAGTCCAGAAGTCCACGGGTGGAC | 3 |
| 99 | AGACTAGAGAACAGTACAGAAAATGCCACCCTAA | 3 |
| 100 | ACGGGTAAGGGAATGCGGGAGTGGACAAACTCTAGA | 3 |
| 101 | AGGACAAATGACGGGATGCCAGGGA | 4 |
| 102 | TGCACGACTGGCAGGATGGCAACGT | 4 |
| 103 | AAGAGGCTACCACGCTCCGGGAGGT | 4 |
| 104 | AAGGCGATCGAAAGAGTAGAGAAGCATACCCGCA | 4 |
| 105 | TAGGACAATAAAAGGGTAAGGGAAGTGGGAGCA | 4 |
| 106 | TCCGAAAATAGAACCCTACAGGCAAGTAGACAGGT | 4 |
| 107 | AAACGCATGAGAAGACCTGGGCGCGGG | 4 |
| 108 | TCGGAACACTGCGCCAGTAAGGCCCA | 4 |
| 109 | TGAAAAACGGTGAGAGATATCACAGAACTGGAACA | 4 |
| 110 | ACTCCGGACATACAGAAATACACGAATAGGGCAA | 4 |
| 111 | TAGACGAGTAACAACGATAACGCGAGTAGCGCG | 4 |
| 112 | AGATACGAAGAGTAGCAAGACTCGGAGGAGTCCA | 4 |
| 113 | AGAAAATAAGCAGATGGCAAAGATAGGGAAGAATAAACGAC | 4 |
| 114 | TGGAGCCAGTGAGGAGCTAAAAGGGTCAGC | 4 |
| 115 | AGACTGGACGGGTAAGGAGATACAAAACTCAGCGA | 4 |
| 116 | ATCAAAAAATACAGGCAAGTACCAGCCATCCACGGGT | 4 |
| 117 | AACGGGATGCCACCGTCCGGCAAAT | 4 |
| 118 | AAAAGAATCCAGCAGCTAGAGCCGGTAACCCA | 4 |
| 119 | ATCCCGAGAGTCGAAGAGATGGCAGAATGAGA | 4 |
| 120 | ACAATACAAGGGATAAGCAGGGTACGGGAATGA | 4 |
| 121 | AACGGTAGAGGACATACGAGGATAACAACACTAAAGGACT | 4 |
| 122 | AGAGAGAATAAACAGCGTGACACGACTAAAAAAAGGT | 4 |
| 123 | ACGGGCGATCAAACAAGTGGAAGACTCCAA | 4 |
| 124 | AAGATAACGCGGTGAGCGAATACGGAGGTCGA | 4 |
| 125 | AAAAAATAGACGGGATGGCGGGATGCCAG | 4 |
| 126 | AGTAGAAGCCCTAAGAGAATAGAAAGCATAAAACACTAGGCA | 4 |
| 127 | AACTTGTACAATAGACGGGTGAAAAGAATGAGGCGA | 4 |
| 128 | TCAAAGCATAGCAAAATCAGGAACTAAGCAGGGTCGC | 4 |
| 129 | CGGCATGGAAACATAGCCAAATAGGGAGGACT | 4 |
| 130 | AGCAGGGTGGAAGGATGACGAACTAGAAGCGA | 4 |
| 131 | TCAACGGATAAGGAGGTGCACAGATAGCACGAAT | 4 |
| 132 | AACGCGGGTAAGAAAGTACAAGAATGAGCAACGTCTAGA | 4 |
| 133 | AGAAGGATACAGGAATCAAGACATGCAGGGAACTA | 5 |
| 134 | AAGACATACCGGAATAAGAGAGGCTAAAGGGA | 5 |
| 135 | TAAGGAGAGTACGAGGAGTAAAGACAGATCGACGGGA | 5 |
| 136 | ATAACAAGAGTCAAGACGTGACCGACCTGAGCGCA | 5 |
| 137 | TCGAGACCGCGGGAGACTCGGCGGGTGA | 5 |
| 138 | ACAAATGGAAGGATAAGAACGGGTAGAAGAACTACGCGA | 5 |
| 139 | ATGAAGGGATGTACAAGGTAGGGCAGATAAGAGGG | 5 |
| 140 | TGGCGAGATGAAAAGGCTCGGAAAACTCA | 5 |
| 141 | ACCAACTGGAGAACTAGGCCGGTCGA | 5 |
| 142 | AGGAATAGAAACGTAAGCGAGATAAGAGGATGGCGC | 5 |
| 143 | AGACTGGGACAATCGGGAGGTCGAGAGA | 5 |
| 144 | TCACGGGAATAGACACGTCCAAGAAATGAACAAGGTA | 5 |
| 145 | AGGACATGCGGGAATAAGAAACTCAAACCCT | 5 |
| 146 | AGGCCAGTCACAGAAGTAACCCAGTACGCA | 5 |
| 147 | AGTCACCGAGTCGGAACGATGACAAAGTAAAA | 5 |
| 148 | AAGGTGAAAGAATGAGAGCACTAAAAAAGTAAAAGAGATC | 5 |
| 149 | AGGCGCGTGGGCGAGATGCAGAGGTA | 5 |
| 150 | ACGAAATAAAAAGATCGCAGAATCCAAAGACTACGGAGGA | 5 |
| 151 | TCAGGCAAATAAGAAGATAAAAAAGATCCGAAGATAACGAGGT | 5 |
| 152 | AGGCCAATGAGAGAATACCGAGCGTAGAGCCA | 5 |
| 153 | TACGAGAGTAAAGAGAGTAGGGCAGTAAAAAGATCCAGCGGT | 5 |
| 154 | AGGAGCATCGGCGCCTCAAGAAGA | 5 |
| 155 | TCCAGAGATGGAACGCTAGGAGAATAAAGCGGTGA | 5 |
| 156 | AAGCGTACAAACGTAGGGAAGTCGGGAGAGT | 5 |
| 157 | AGAGAGATACAGAGAGTAAGAGCCATAGACACCTGAGACGA | 5 |
| 158 | TCGGCAACTGGGCAACATCCAGAGA | 5 |
| 159 | TGGGCACCTAGGCACATCACCGGGTGCA | 5 |
| 160 | AAGGATCACAGAGTAGAACGCTCAAAGAAGTCACCA | 5 |
| 161 | AGTGCACGGGTAAGGGACATGCGA | 5 |
| 162 | AGGTGAGAGCGGGTGGAAAACTCGAC | 5 |
| 163 | AGCTCAACACATGAGGGAATGCCAGAGA | 5 |
| 164 | TGGAGCAAATAAGAGACATGCCGGGCGA | 5 |
| 165 | TGAGGAACTACAACAGTAGCCCAATGCA | 5 |
| 166 | AGGCTAAGGAAGTACAAAAGTCGGGAAAGGATCC | 5 |
| 167 | AGAATGAAACAGTGGAAAGGTCGGAAACTGAGGCGG | 6 |
| 168 | AGTAGAAACAAGTCGCGAGATACGAGAGATGAA | 6 |
| 169 | AGAGCGTCAAAGAATGGAGGACTCAGAAGAT | 6 |
| 170 | AGAAACATCGGGACAGGTGAGAACACTGGGA | 6 |
| 171 | AAGAATCGAAAAAACTACCAAAACTACAAAAACTGCAAAA | 6 |
| 172 | ATGAAAGGAGTGGACCACTGGACAGCCGCGGA | 6 |
| 173 | AGCTCAAGAACTGTGTACAGGTGCAGCGG | 6 |
| 174 | TCAGAAACATCAAAAGGTAACAGCATCGCGGGA | 6 |
| 175 | TCGGAAAATCCGGGAGTCAAAAAGTGCAGAA | 6 |
| 176 | ATGACACGGGTACGAAACCTCAGAAACATAAACAGCT | 6 |
| 177 | ACGGGCATGAGAGCATAAAGAGATAAAAAAATGC | 6 |
| 178 | AGAGCGATACCACACGTAAGGAGATCGCA | 6 |
| 179 | AGGTGCCACGATCAGAAAATAGAGACATCGACGGG | 6 |
| 180 | TGGCGAACTGAGCAACATCCGGGAAT | 6 |
| 181 | AAGAACCTCAAGGGCGTGCAACCGGT | 6 |
| 182 | ACACGAATGAGAGGATGCGGGAGTCAGCGG | 6 |
| 183 | AGTGGGAAGGTGAGGAAAGTAGAAAAAATACAGCC | 6 |
| 184 | AGGTACAAAAGTAAAGCGGGTCAACCGGT | 6 |
| 185 | AGCCAAGGTGCGAAAGTAGGCCCGTG | 6 |
| 186 | AGAAAATCCGGCAATAGAACAGTAAAAAGGTGAGACGCA | 6 |
| 187 | TAACACGGGTGCAAAGCTAAGAGAGATCCACC | 6 |
| 188 | AGTAGGAAGATGGGAAAGCATAACAAAGATAGAAGAATAAGGC | 6 |
| 189 | AGTAGGAGAATGAAAACAATACAGAAAAGTAGGGCAGA | 6 |
| 190 | TAAAGCACTAACGAGCTGACAGGCTGGCAGGA | 6 |
| 191 | TGAAGACATGACACGGATCAAGGCATGGACG | 6 |
| 192 | AGTGCACAGGTCAAGCGCTCCGGCGA | 6 |
| 193 | TGAGCGAATAGCCCGCGTAACACAGTCCG | 6 |
| 194 | ACAAGGTGGGAGCATAGCCCGATCACG | 6 |
| 195 | AGGGTGCAGGCGTGAGAAAAGATCCAGG | 6 |
| 196 | GCTAAAACACTAGAAACATGGCACAGTACAGGGACTG | 6 |
| 197 | AGGAGCTGACAAGATGCACACGGTCAA | 6 |
| 198 | AGGATAAGAAACGTCACGAGGATACCACCATCACGAAA | 6 |
| 199 | TAAGCGGGTCGGAAACATGCCAGACTGGGCAAG | 6 |
| 200 | TCACCAGGATGAAGAAATGAAGAAGTAGCAAGAGGATCC | 6 |
| 201 | AGAAAAACATAAGCAGCATGGAGCGCTACGGG | 7 |
| 202 | AGGATGGCACGGTCGCGGGAATCGA | 7 |
| 203 | ACGCTGAAGCAATCAAAGGGTAGGGAGG | 7 |
| 204 | TCGGGCAATACAGCGATCGGAGGATGAG | 7 |
| 205 | ACGGGTCACAAGAAATGGGACCATCCGCA | 7 |
| 206 | AATCAAGAGATAAGGAAATACAAGGAATAAGAAGGATGAAAACG | 7 |
| 207 | TGCGGAGATGAAGACGATGACGACAATAATGTACA | 7 |
| 208 | ATGAGCAGATACACACGGTGAACCCCGCGGA | 7 |
| 209 | ACGCTAACAAAATACCGAAATAAAAAAATAGCGAGATACAGGGCT | 7 |
| 210 | AAAGAAGTGCAAGGGAGTAAGGCCCTGGC | 7 |
| 211 | AGGGTAACAAGCTCGCGGACGTCACCGC | 7 |
| 212 | CGTGCGGGCAATACGACCGCTAAAGAAGCT | 7 |
| 213 | AGAGAGAATCAGCAGGGTAAAGAAGTAGGACCG | 7 |
| 214 | ACTCGCAAGATCGAGAAATAAAAAAGTGGACCGGG | 7 |
| 215 | TGAGCACGGATGAACAAGTGAAGAAATACAGAGGT | 7 |
| 216 | ACAAGAATCAGAAACCTCGGGACATAGAAACATCCA | 7 |
| 217 | AGAAGATGGGCAGAATCGAACAGGTAGACGAGTG | 7 |
| 218 | AGAAGACTAAAACAATGAAGAACTAGGCCCGCAT | 7 |
| 219 | ACGAGACTGAAAGCATCAAGAGGATAAGGAACTCAGA | 7 |
| 220 | AAACTAGAGAAACTAAGGCGATCAGGAGGATGAGAA | 7 |
| 221 | AATCGACGAGTAAGGGAAGTCCGGGAGT | 7 |
| 222 | ACACGGATGAACCGATACAAGGATGGCGACG | 7 |
| 223 | TCCAGCAATGGAAAGGCTCAGCCGAT | 7 |
| 224 | AACACGGTAGGAGCAATAGAAGAAATAAAGACGTGCG | 7 |
| 225 | CGGAATAAGAAAGGATAACGAGATCAAGACAATGGAACGAGT | 7 |
| 226 | ACAAAAGTCAAAACGTCGAAAGGGTAAAGCACTGACGA | 7 |
| 227 | ACATAGAGCGATAAACACCTGGGAAGATCCGGA | 7 |
| 228 | ACTAGCAAAATCACGACAAATGCAAAGATCAGCCGA | 7 |
| 229 | TCGAGGCATACCAGGGTGAAAAAATCAAAAAAGTA | 7 |
| 230 | ACCGGACCTAAACCAACGTGAGAAAATGCG | 7 |
| 231 | AGCGTACAGACCTGACGAAATCAACAAATGA | 7 |
| 232 | AGAAGTAACAGAGATCCAAAACTGAAAAGGTAAAAGCA | 7 |
| 233 | TGGACACGTAAGCAAGATAGACAAGGGGATCC | 7 |
| 234 | AGAGGTAGCACACGGTGAAAAGCTAAGAACCTCA | 8 |
| 235 | AACGATCGCACCATGACGCGAATGAGACAA | 8 |
| 236 | ATGCCGAAATACACACATACGAAACCTCAAGACCCTA | 8 |
| 237 | ACGGGCTAAAAGAGTCGGAGCCATAGAAAGGT | 8 |
| 238 | AACAGCGATACCGAAATGGAACGGGTGC | 8 |
| 239 | AGCAATAAAGAAGCTAAACGAAGTAGGAAAATAGGGAA | 8 |
| 240 | ATGGAACCAGTGGGACATGTACAGACGAA | 8 |
| 241 | TCGGAAACATGACGACCTGGACGGGT | 8 |
| 242 | ACGGCAAGTAAAAAAATAAAAGAGTAGAGACATGAAAACAT | 8 |
| 243 | ACCGCGGTGAGAGCGTAGAAGCGTGA | 8 |
| 244 | AACAGTAAGCGGCTAAGGGAGTCGGAGGA | 8 |
| 245 | TAAACGGCTGACGGAGACTGACAACCATAAAGC | 8 |
| 246 | AGGTGCAAAGGGATGGCAACGTCAAGGCG | 8 |
| 247 | TCGCAGAAGTAAGGGAATGGCAAAACTAAGCAAAGT | 8 |
| 248 | AGAGCACTAACAAAGGTAGGACGAAGTACGAAGG | 8 |
| 249 | TGAGGAGGTGGAAAGCTGGCGCACA | 8 |
| 250 | TGAGAGAATACAAGAACTAGCGAAGGTACAGCACTGG | 8 |
| 251 | ACGGATCAAAACGGTCGCAACGTAAGACGGT | 8 |
| 252 | AGGGCGGTAGAAAAGCTCAACGACTGAGAGGCA | 8 |
| 253 | TGAGGACGCTAAAGGGAGTAGCAAGCTGAGA | 8 |
| 254 | AAATGCCGGAAGTGAAAAACTGGAAAGATAAGCAAG | 8 |
| 255 | TCCGCCAAGTCGAGAACGTAGAAGAGTACGGG | 8 |
| 256 | AGTACAGAACCTGGGCGAAGATCAGGCGAGT | 8 |
| 257 | AGCAGAGTCAAAAACTGGCAAGATCAGGAGCTA | 8 |
| 258 | ACCAAGGCTCAGGCAATGCAAGACTGA | 8 |
| 259 | ACGGGTCACAAGCGTCAGGACATAGAAGA | 8 |
| 260 | ATGAAAGAGTACCAGAATACACGGGATAGCAGACGT | 8 |
| 261 | AAGGACGATAGGAGCGTGAGCCAATCCG | 8 |
| 262 | AGGATAGAGGAATAAAGCACATAAGCGGGTGAGACCA | 8 |
| 263 | TGAGAGGATCGAAGAATACGGGAATAAGGCGGGT | 8 |
| 264 | AAGAACGTGACGCAAGTGAGGCGATAAGAACAA | 8 |
| 265 | TAGCGCGAATGACGGAGTACCCAAATCAAGGGA | 8 |
| 266 | TCGCCGGGTGAAAAAGTAAGAGAATCGCCAGCGTCCA | 8 |
| 267 | AGGAAGTAGAGGAATACAACAAGTCACGGAAGGATCC | 8 |
| 268 | AGATCGGGAGATAAAGGGAGTAAACGCATCAAACGA | 9 |
| 269 | TGGCGAGATGAACAGGATGACAGGGTCCCGA | 9 |
| 270 | ACATACCGGGATGGAGCGCTGGGA | 9 |
| 271 | ACCTGGAGGAACTAGAAACCTCCGAAGCTCC | 9 |
| 272 | ACGGGTGAACAGGTGGACAGGTAAAAGAATACAACA | 9 |
| 273 | AGTACAACCGTGAAAAACCCTCAGGCGGT | 9 |
| 274 | AGGGAACTCGAAGAAATGAGCGGGTAAGGA | 9 |
| 275 | AGGTAAAGGGATGAACGGGTGGAAAGATGGGA | 9 |
| 276 | AGGTAAAGAGAATGGGACACCATAACCGCAT | 9 |
| 277 | ACGCGGAATGGCAAGAGTGCACAACT | 9 |
| 278 | AGGAGAAGTCACAAAGTGGAAAACTCGCACCGT | 9 |
| 279 | AAGGACCTGGACGGGAATAGACGGGA | 9 |
| 280 | TCGAACGGATATCGAGTACGGAAAGTCCAAGAGC | 9 |
| 281 | TGCGACACTAGAACACATGCACGACTACGCC | 9 |
| 282 | AGGTGGAAAGCGTAAACGCCGTGCAA | 9 |
| 283 | AGCTCGCCCGGAATCAGACAGTGGGCGGA | 9 |
| 284 | TAACGAAAGTAACCCAGGGTGACGCGC | 9 |
| 285 | TGCCAGACTAAAGGAGTAAGGGAGATACAGGCACT | 9 |
| 286 | AAAAGGGATAAGCGGATGACACGGATAGCAA | 9 |
| 287 | ACGTAAGGAGATGACAGGCATAGCAGAACTACGA | 9 |
| 288 | AAATGAACGAATAGAGAGGCTAAGAGGGTGGAGACGA | 9 |
| 289 | TGCAAAGAATAAGAGAATAAAAGACGTGCAGGCATCGA | 9 |
| 290 | AAACTGCAAGAGTAGGGCCATCGGCA | 9 |
| 291 | ACTCGACCGGGTACAACGCTAACGAACGT | 9 |
| 292 | ACAGGGCTCGGCGAATCGACAAACTCGA | 9 |
| 293 | AGGATCAGACCACATGGAAACGTCGGGACA | 9 |
| 294 | ATAACGGAATAAGCAACTACGACGGTGAAGGCGTCA | 9 |
| 295 | ACAAGTGGACGAGTAAAGGCGTGAAGACG | 9 |
| 296 | TCCAGAGCTGGAACCCTGCGGGCAT | 9 |
| 297 | AAAGAGAGAGCTCAATGCGCAGACTGAAACAA | 9 |
| 298 | TAACCAGGTAGAGGAGATCCACCGGTCAGGCGA | 9 |
| 299 | TAAAGAAAGTCGACAGGTGACGAGGTGACGAGGT | 9 |
| 300 | ACGCGAATAAACCAATACCGGAATGAGAAGGT | 9 |
| 301 | ACAGGCGTAGAAAAGATGAGGAGATCAGAGCGATGAAGAGAT | 9 |
| 302 | AGCGAAGTAAAGAAAATACGAAAGTCCGGGAGAACTCGAG | 9 |
| 303 | AGAGCTACAAGACTACAAGAGTCGCGCGCCGTA | 10 |
| 304 | AAAAAAGTGGCGCAGGATGGAAACGTGAGG | 10 |
| 305 | AGGTAGCGAAGGTACAGACCTGGAGAGATCAGGA | 10 |
| 306 | ACTGGCAGAATGAGGACATCAGCCGATGA | 10 |
| 307 | ACGCCTGGGCAAATAACGCAATAGAAGAGT | 10 |
| 308 | AGGCAGGGTAGAGCGCTAACGGAATCCGC | 10 |
| 309 | ACGATAAGAGCGTGGGAGGCTAACAGAATGGC | 10 |
| 310 | CCAATGACGGGACGTAAGCAGATGAAAACAGTGGCGG | 10 |
| 311 | AGCTGGAAGAATCAGAGCAGTCCGAGGG | 10 |
| 312 | TGCAGAGGTAAGGAGATGGGAGAAATGCAAAGA | 10 |
| 313 | ATGCGGAAGTAAACAAATAAGAGGATCGGAGGGCTAA | 10 |
| 314 | AACGGGTCGGCGAGTGAAAAAATCGGGA | 10 |
| 315 | AGTCAGCGCATAAAGGCCTGAAAAAGATAAGCCGA | 10 |
| 316 | TGAAGAAACTGGGCGGATGACGCGGGTGA | 10 |
| 317 | AAGATATCCGGGAATGGCACGGTGGCGGGA | 10 |
| 318 | TGGAAAAGTGCGGGAATCGGGCCGTGA | 10 |
| 319 | ACGAATAGAACGGGTACACGCGATAGGCA | 10 |
| 320 | AGATAACCGGATCGAGAGCACTCAAGCGAT | 10 |
| 321 | ACCAAGCTCGCAAGAGTAGGAGCGT | 10 |
| 322 | ACCGGGAGTGACAAGATAAGAGAGTAGGGAGA | 10 |
| 323 | TCCAGAAGTCACGACATCGGGAGCTGCCGGGA | 10 |
| 324 | TCAGAGGGTCAGGGAATAGCGAAAAATGCAAGAA | 10 |
| 325 | TAGAAACATGCAGACATGAACCGGGTAAGCCAA | 10 |
| 326 | TGAACCACTGACAGAGGTGAGAGCATAGCGAGCT | 10 |
| 327 | AGGACGGTAAAAGAGGTCCAACGCGGTCC | 10 |
| 328 | AGAGATCCGAAAATCGAAAGAGTAAAAAGATAGGAAGGTG | 10 |
| 329 | ACCCACTGCGAGGGATGACAACGTGAAAAA | 10 |
| 330 | AATGGCGCGATAACAACAGTACGAAAGCTACGGGA | 10 |
| 331 | ACTGACAGGGTCGAGCTCTAGCAAAACTGCGGG | 10 |
| 332 | AGTGGAACGGTAGAGGAGTAGGGCAATAGAAACA | 10 |
| 333 | TGAAGCAATGAAAGCCGTAGGGAAACTCCACC | 10 |
| 334 | AGTCGGAGAATACGACAGGTCACGCAAATAAAGCCGTCA | 10 |
| 335 | ACGGGTGACCGGCTACAAGCGTGCAACAAAATAAGAACAGTGGG | 10 |
| 336 | ACAGAGTAGGCCACTAGAACCATAACCAAACTCGAG | 10 |
| 337 | AGACTACAAAAATGCACAGATGGCGAGGTAAAAAGGT | 11 |
| 338 | AGCCAAGCTGCGCGGGTGAGCGGA | 11 |
| 339 | TGGCGAAGTGCACAGGTAAGAGAGTACAC | 11 |
| 340 | AGCTCAAGCAGTAGGGCAAGTAAAGAACTCACA | 11 |
| 341 | AGATAAGCCGATCAAGGCGTCCAAGCG | 11 |
| 342 | TGGCGAGGTGGGAGACTACAACACGAT | 11 |
| 343 | AGAGCAGGTAAAAAAGTAAAAAGATAACAAAAATCCGGGA | 11 |
| 344 | AGTGAAGGAATCAAACAATGGAGAAGTGAGGCAAC | 11 |
| 345 | TGGAGAACTCAGAGCATACGGCAGATGGA | 11 |
| 346 | ACGGTCCGGGAAATCAAGCGAGTGAGAGGGA | 11 |
| 347 | TCGGGAAATAGGCAGAATCAAACAAGTGGGA | 11 |
| 348 | AGATCAACCGGTGAGGAGACTAAACGCAT | 11 |
| 349 | AACCGGATAGAGCCGCTACGGCACTA | 11 |
| 350 | AGACCGGTAGGGAGCTGCGGGAGTGCGAGACA | 11 |
| 351 | TGGACGAGTGGACACATCGAGAGGTCA | 11 |
| 352 | AGGAGATAGAGAGGACTGAGGGACTCCCAGGAT | 11 |
| 353 | AAAAAGGAGTAAGAGAGTCCGGCAGGTCCCAGAT | 11 |
| 354 | ATCGGACGTCAAACGATAAAAAAATGGGCGGAT | 11 |
| 355 | ACAGACGGGTCAAGAAATCGCACGGC | 11 |
| 356 | TCGGACGATGACGGAATAGGAAAATGAGGCGCTAA | 11 |
| 357 | AAAACTAGAGAAATGAGGAAGTACGAGCGTCGCGGA | 11 |
| 358 | ATGAGGAAATACAAGGATAGGCAAATGAGACGATGGAGGCGCA | 11 |
| 359 | TCGACAAGTCAGAAAAATGCCGCGATCGAGG | 11 |
| 360 | ACGTCGACAGAGTAGGACACTGCGGGAAA | 11 |
| 361 | TAAAGGAATCAGAAGGTAGAGAGCGTCACGCGGTGGA | 11 |
| 362 | AAGCTCAGCGCAATCACGGACTAGAAGGA | 11 |
| 363 | ATCCGGACATCGAGAAACTACGAGAAGTGAAAAAGT | 11 |
| 364 | AAGCCGATCCAGACATGCCCACAATGGC | 11 |
| 365 | ACCATAACAGAGCTCAAAACTGGCGAGGAGTG | 11 |
| 366 | AGCCACAGTAAGACAGTCCCGAAATAAACACAT | 11 |
| 367 | ACAAGGCTAGAAAAATGCGAAGAGTGCGAGA | 11 |
| 368 | AGGTCGGGAAGGTAGAGAGAATACAAGGCTGACGGAGTGAGGA | 11 |
| 369 | AATAAGAGAGTGGACAGAGTACGAGAGAGTGCGACCAA | 11 |
| 370 | TAAACGGGTAGGCGAATCAACGGATAGGAGAACTCGAG | 11 |
| 371 | AGAGCCAGTCAAGCAATCGGGAGAATGGC | 12 |
| 372 | ACCGTGGGCGGCTAAGCAAAGTACGAAA | 12 |
| 373 | AATAACAACATGCGGGAATCGGAGCGTCC | 12 |
| 374 | AGAAGTCAAGGAATGGCAAGGGTGCGCAA | 12 |
| 375 | ATGGAAAGGTAAGAGGGATGAACCCATAGAGAAGG | 12 |
| 376 | TGACGGGATGAGAACGCTAAGAAAATCCCAAAA | 12 |
| 377 | TAGGAGAGATAGGAGGGTGGACGAAATGCCA | 12 |
| 378 | AGATGACGCAATGACAGAAGTAACGGGAAGTGAC | 12 |
| 379 | AGACTGGCCCACAATGCAGAGGTAAGGC | 12 |
| 380 | CCTCAACCGAATCGGGCGATGAAACACTGA | 12 |
| 381 | AAACGTAGAAAAGTGCAGCGACTAGCAGGAGTAA | 12 |
| 382 | AACGATGCCCGAATAAAAACCTAAAGGAGTGGGAGA | 12 |
| 383 | ATCCACGAGTAAGAAAATCAAGGAGTAACCGACTCAGA | 12 |
| 384 | AAGATGACCAAGGTCAAGGACTCAAAAGCTCGG | 12 |
| 385 | AGAGTAACCCAGCGTCACAAAATGAGAGCC | 12 |
| 386 | TGCAAAAATAGAAACATGCGGCAACCTGAAACGC | 12 |
| 387 | TGCCAGACAGTGGGAACGTCCACAGG | 12 |
| 388 | TGGAGGGATGGGAAAGTACAACACTGACCAGA | 12 |
| 389 | TCGGAAAATGGCAACACTACGAAGGTGGATATCT | 12 |
| 390 | AGCAGAATGAAAAGGGTAAAGAGACTAAAGCAATCCAGA | 12 |
| 391 | AGTACAACGATAGGAGCGTACGAGAATGCAAAGA | 12 |
| 392 | TGAACGGGTACGACAAGTGAAAAACTCAAGAGATACA | 12 |
| 393 | ACCATGCACGCGATAGACACGTACAAAACTACAAA | 12 |
| 394 | AATGAACACACGTAGGAGAGCTGAACAAAGTAGGC | 12 |
| 395 | CGCATGGAGAAGTACGGCGGCTGGC | 12 |
| 396 | AGAATGAAGGCGTAAGAGCACTAAGCGGAGA | 12 |
| 397 | TGGAGACATAAGCACATGGGAACGTCAAAAAATCAG | 12 |
| 398 | AGAGTGCAAACATAAACACATGAGCTCATGCGAG | 12 |
| 399 | AGTAGCACAAATCGAGAGGTAGAGGCGTCG | 12 |
| 400 | AGGGAGTAGCGGAGTACCCAACATGGACC | 12 |
| 401 | ACTGGAAAACTCAGGGCGGTGGACGGA | 12 |
| 402 | TGAGGAAGGTACACGGGTAGAAACATAGCGCGA | 12 |
| 403 | TGAGAGCAATAAGAAAGGTGAAAGCATGAGAGACTACAGAAGA | 12 |
| 404 | TGGACACGATAAAAACGTAGGCAAGCTCGAG | 12 |
| 405 | AGACCCACTAAGGCAATGAAAGCCTAGAGGCA | 13 |
| 406 | TGACGGGATAAACGGGCTCGGGAAGC | 13 |
| 407 | TGCCGGGCTGACGAGAATGGAGGCCTA | 13 |
| 408 | AAGAAACTGGAGCGATCAGACAGGGTACGACGC | 13 |
| 409 | TCACGCAGGATGACAGCAATACGACGCT | 13 |
| 410 | ACAGGAATGGAAAGAATGCGAGAGCTAAAACAGTCCA | 13 |
| 411 | AGGGTAGAGCGGTAGAGCTCTAGGGAACTA | 13 |
| 412 | AGAAGAGATGGAGAAATAGAGACGATAGAAACCTAGCAAA | 13 |
| 413 | ATCCGAAGCTCGGGAGATCCAGCGAG | 13 |
| 414 | TGAGGAGATACACGAATGCGAGCGATGGCGA | 13 |
| 415 | ACTGCCAACGGCTGAACACAATGAGCAAATGGAGA | 13 |
| 416 | AATAAGCGAACATAGGGCGATAAGAGACCGCGGCA | 13 |
| 417 | ACGTCGGGAGGTCAACAAGTAGAGGAATAAACC | 13 |
| 418 | AGTGGGAAAATCAGAAAATAAAGAGGATAAAGGCGTCAGGA | 13 |
| 419 | AATGGGAGGAATCGGGAAATGAACGCGTAAGA | 13 |
| 420 | AGATAGAAAGGATGCCGAGGAGTCAACCGAA | 13 |
| 421 | TGACAGACGTAGGGAAAGATACAACAATCACCAAATCAAA | 13 |
| 422 | AAGTGCGAGCAGTCGAGGAATCGGGAGGT | 13 |
| 423 | AGAAGGAATGACAACGATGAAGAACATCAAAACGTGAC | 13 |
| 424 | ACCCTAAGGCCCTAGAGCGATAAAAGAGTGAGGCA | 13 |
| 425 | ATCGAGGGATGCGCGCGTAGACA | 13 |
| 426 | AGTGCGAACGTAGACCACTAAGAAAGTCAGCAGAA | 13 |
| 427 | TAAACAGAGTAAACCACTAGCAAGATCAAAGACTAAAAAACTACGC | 13 |
| 428 | ACCGTAGCCAGACTCGGGCAATGA | 13 |
| 429 | ACGGATCAAAAGATAAGGGAAATGACAGGATGCAGA | 13 |
| 430 | AGAATAACGGGACTACAAGGCTAAGGCAGTCAGA | 13 |
| 431 | AAGTAACGCACTGGCAGGGTGAAGACCTGCA | 13 |
| 432 | ACAGTGAAAGGGTCGGGCAATAGCAGA | 13 |
| 433 | AGTGACCACATACGCCAATCGACAAGTAAAGAGGT | 13 |
| 434 | AAAGCAACTAACGAACGTACAAGAAATAACCGGCTGAAAGGA | 13 |
| 435 | ACTAGCGAAATGAGGGAGTGAAGAAATAAGCAGAACT | 13 |
| 436 | AGGGAGCTGCGACGGGTGGCAGAGAGTCGAGAGA | 13 |
| 437 | TACAAAGGTAAACAAACTCGCAAGGGTGAAGAGACCATGG | 13 |
| 438 | AGACGATAAGCAAAATAGGGCCGTGAGACAA | 14 |
| 439 | ATGGCGACATGAAGCAATACCCAAGTGACA | 14 |
| 440 | AGCTAGAGAGGTAACAGCATAGACAACCCTAACGGG | 14 |
| 441 | ACTAGCCCAAATAGAAGAGTAAACGGGTAGGGA | 14 |
| 442 | ACTGAGGACCTGAAAACCTGCAAAGACTGGGC | 14 |
| 443 | AGGGATAGGAACAATAAGAAGATGAACAGATGAGCGAGC | 14 |
| 444 | TCGGGAAATGAGAAAATAAAAGGCGTACGGGAGTGGGAC | 14 |
| 445 | AGTCACGAGAATAAAGGCGTCGGCAGATCA | 14 |
| 446 | ACGGATGCAGGGCATGGGACAGTACGGG | 14 |
| 447 | AGAGTACGCGGCTGAAGAGCTGACACCC | 14 |
| 448 | TGAGGGAAGTAGGCAAATAAAAGGGTAGCCCACT | 14 |
| 449 | AGCGAGCGTCACCGGGTGGAAAGCT | 14 |
| 450 | AGGAACGTCGGAAACTAGGAGAGTCAGCAGC | 14 |
| 451 | TCCGAAGCTGAGAAACTCGGCAGATCACGGAC | 14 |
| 452 | CGCGGGAAGATGAAAAGCTGAGGAGGGTGG | 14 |
| 453 | ACGGGTCAGAACGTGGGAAACTAGACGACG | 14 |
| 454 | TGGGCGAATACGCACGTAAAGGAGTACGACACA | 14 |
| 455 | TCAGGGCCTGGGAAGATACCAAGATGCCGGA | 14 |
| 456 | AGATCGAAAACTAAAGCAGTGGAACAGTCAACAAATCA | 14 |
| 457 | AGGGCGATAAGCGAATAAGGAGGTCAGCAGG | 14 |
| 458 | TGGAAACCGCTAAGACCGTGGGAAACTC | 14 |
| 459 | AGGAAATACGGGCAGTAAGGCGGCTGGC | 14 |
| 460 | AGACTAGCGACATGAGCGGGTCACAGA | 14 |
| 461 | AGGTAACGCAATAACAAAATCGCGCAGTGGCAC | 14 |
| 462 | AGTAAAGGCCTCGGGAAGTGCGGAGA | 14 |
| 463 | TGCAAGAGTACCAAAGGCTAAGGCACTGGGC | 14 |
| 464 | ACGATACGGGAACTAGGGCGACTGACAGCA | 14 |
| 465 | TCCACGCAGTAAGAGAATGGCGGGA | 14 |
| 466 | TGGAGCGCTAAGACGGTGAACCAATAAGGGCCT | 14 |
| 467 | AACAGGATGACAAGGATGCGGGAATGAGCCACTA | 14 |
| 468 | AAGGAAGTAGAGGAGTAAGCCGGGTGCGA | 14 |
| 469 | AGCTGGAGGGAATAGGAAAATACGAGGATGGG | 14 |
| 470 | CAGGTGAGGAGAAATCGGACGGATGAACG | 14 |
| 471 | GCTGGCAAGGTAAAAGGGTAGCGGAAT | 14 |
| 472 | AGAGAGCAGTCCGCAGGTCAAACGGG | 14 |
| 473 | TGCACGGGCTCAGACGGGCCATGG | 14 |
| 474 | AGAAAAATGGCAAGCTAAGAGGAATCAAGAACTGCCC | 15 |
| 475 | ACCTAAGACCAATGAGGCGATAACCGAAATCGGG | 15 |
| 476 | CAATAGCCGAATAAAGGGAATGAGACGGGTGCG | 15 |
| 477 | CGACTACACAAGTGCGCAACTAAAAAAATAACGAAGTGGGA | 15 |
| 478 | AGCGCTACAAAGATGGGCAGATCGGC | 15 |
| 479 | AGGTAAGGACGTAGGGAAGTACAGGAGCTCCG | 15 |
| 480 | CGACTAGGACCATCCAACACTGGCAGGAT | 15 |
| 481 | AGAGCGGTGAAAAGCTGGAGAAACGTCGGG | 15 |
| 482 | ACGTGGGCAGGTCAGAGGGTGCAGA | 15 |
| 483 | AGTGACGGGCATAAGCACATACAACGGTAGC | 15 |
| 484 | AGAGTCGAAAACATAAAGAGACTGGACGAATCAGAGACT | 15 |
| 485 | AGCGGACTAGCCACCTGGGAGAGT | 15 |
| 486 | ACCCGGGATACAAGGGATAAGAGGAATAGGCG | 15 |
| 487 | AGTGGACAGATGGAAGCATGGGAGGATCACAGA | 15 |
| 488 | ACTAGGGAGATAGCGAGATACCAGCGTGGAGA | 15 |
| 489 | AGTAAAAAAATGAGGGACTAAGGGAATGAAAAAGTAAGAAACC | 15 |
| 490 | CGCGGGGTACACCAGTGCAGCAGT | 15 |
| 491 | AGGAGAATACACGAATGCAGCCAGTCAAGAGAA | 15 |
| 492 | ATGAAGAACTAAGAGAGTGCGAGAGTACAGAGCTACGCA | 15 |
| 493 | AGTGCCCAAGTGAGAGAATAGCGGGCCTCA | 15 |
| 494 | AGCGATCAACGACATAACAGGAGTCAGGAGAA | 15 |
| 495 | TCGCAAAGTCACGGGATGCGAGCAGTGG | 15 |
| 496 | ACAAATCAGCAAAATCAAAGACTCACAAGATCCGACAA | 15 |
| 497 | TAGAGGGATAAACAAGATACAAACATCCAGAGACTGGC | 15 |
| 498 | AGGATAAAGAAAGTACAAAGCGTGCCAAGCTAAGGA | 15 |
| 499 | AGTAGAGAAATCCAAGAATACAGAGGTGACGCCGTGA | 15 |
| 500 | AGACATGCGCAGGTGAGCAGGATAGG | 15 |
| 501 | AGACTAAAGGCGTACGGGAATGCGAAACT | 15 |
| 502 | AGAAGGCTGAAAGGATCGACCCACTCGC | 15 |
| 503 | AGCGTAGAGGGCTACGACAACTAAAGACATAAGCAGA | 15 |
| 504 | TGAAGCCCATCAAGGACATGGCGCGA | 15 |
| 505 | TGGGAAGATCCAAGAGATCCAAGCCCTAGGAAAGATGA | 15 |
| 506 | ACGCCTGAACAGCTAAGAGCGGGTCCA | 15 |
| 507 | AGGATAAGAACATGCAGAAATGGACGAGCCATGG | 15 |
| 508 | AGACCAAAAGTCAAGAAAGTACCGGGCTAGAAGAGCTGA | 16 |
| 509 | AGCCATAAGCGAGTAGCAGAATAGAAAGATCCCAA | 16 |
| 510 | AAGTCCCAGGGATAGACGAGTAGGAAGGTGAA | 16 |
| 511 | AAAATGGCGAGGTAGCGACATGCAAAGGT | 16 |
| 512 | AAAACGATGAAAAACTACGAGGGTGGAAGAATAAGC | 16 |
| 513 | AGGTGACGAAGTAAACGGGTCAAACCGAGCTC | 16 |
| 514 | AGATCGAACGATAGGAAACATGACCGGGTCAC | 16 |
| 515 | ACGATCGAGAGGTCCCAAAGGATAGAAGAAGTGA | 16 |
| 516 | AAAGGTGAGCAAGGTGGCGAAAATAAAAAGATA | 16 |
| 517 | AAAGAATAAGACAGTAGCGGGAATACGACACTAGA | 16 |
| 518 | AGGATCGGGACATGCAGCAGTAAACCAA | 16 |
| 519 | TAGGAGGATAACAGGGCATGGAAGAGTGGGACGGT | 16 |
| 520 | AAGACCCTACACGAATACAAGCAGTGCCAGGA | 16 |
| 521 | TGGCGCGAGTGACAAAAAGTAGAAGGGTG | 16 |
| 522 | ACCGAGTCGAGAGATAGAGACGTAAGGAAGTAGGGA | 16 |
| 523 | AGGTGGGACGATCGAAAGATCGAAGAGT | 16 |
| 524 | AGGAGGCGTCGAAAAATCCGGAAAATAGGGA | 16 |
| 525 | AGATGGACGGATCGGGACGGTGAGGAGGA | 16 |
| 526 | ATAGCAAAAGTCCCGCGGGTACGAAAGGT | 16 |
| 527 | CGGGAAGGTCAAGCAATCAGGCGCTCA | 16 |
| 528 | ACGGGACTGAACAAATAAGGACATACACAAGTCGGC | 16 |
| 529 | ACGTCACGAACTCAAAAGGTGGAGAAGGT | 16 |
| 530 | AGCGAAATCGAGGAGTGGAGAAGGTAAAGAA | 16 |
| 531 | ATGGGAAGGCTAAAGAAATGGCAGGGTAGAGA | 16 |
| 532 | ACTGGGACGGTAAACGCATGAAAGAATCAGGGAGT | 16 |
| 533 | AGAAGAACGTGAAGGGATAGGAGAACTCAACAGGGT | 16 |
| 534 | AGCAGAAGTGGAAAGCATGGCAAGAATGGCAGCA | 16 |
| 535 | TGAAAAGATCCAGGAGTAAGCGAGCTGAAGAA | 16 |
| 536 | ATGGAGACGTAACAACATAGCGGGAGTAGGCGCGTGACA | 16 |
| 537 | AGATAACGCGAATGCGGAGGTCGAGGAA | 16 |
| 538 | TCCGCAAGTGAACACGTCAACGCAATGA | 16 |
| 539 | ACGGATGAACACATGCACGAAGTCGACAAGTAAA | 16 |
| 540 | AAACGTGGAAGCCATGACAACATAACGGGA | 16 |
| 541 | TGGCAGGATAAGAGAGTAGAACGATGCACGAGCCATGG | 16 |
| 542 | AGACAGCGATCAGAGGGTAAAACGGGATGA | 17 |
| 543 | AGCAGTGAAAGGACCTCAGCGAATGAAAAACGA | 17 |
| 544 | TGGCCAGATCCAAAGATAAAAAACTGAAAGACTACGGAA | 17 |
| 545 | ATACAAGAATAGAAGGGTAAACGACTGAGAAAGTACGAAGCCT | 17 |
| 546 | AGACGGGTAAAAAAGGTCGGGAAGGGTAACGCCA | 17 |
| 547 | TAGACAAATGAGAAGGTAAAGGCATGGAAAAAATGGAGGCA | 17 |
| 548 | TCGACGAATGCCCGGCTCAAAGGATA | 17 |
| 549 | ACGGACTAGCGCGGTAAAAGGGAATGCGG | 17 |
| 550 | ACGATCGAAGAAGCTCCGGACCGATCC | 17 |
| 551 | ACGGAATAGAGACATACGACAGTGCGCCAA | 17 |
| 552 | ATGGACCGATAAAAGGGTAGACGAAATAACAGGATGA | 17 |
| 553 | ACAGGACTCGGAGAAATAACAAAGTGGAGAAAGTACAA | 17 |
| 554 | AAGTCAACGAATAGGCCAGTGGCAAAAGTGAGCG | 17 |
| 555 | AGTGAACAGGTAGAGGAGTGGAAAAGTACAAAGGA | 17 |
| 556 | TGCAAAAATGAAAAGGTAGAAAACTAAGGCAGTACAGGCAT | 17 |
| 557 | AAACGACTCACAAACTAGAAAACTACAGCAGATCGAAGCAT | 17 |
| 558 | AAAGGAAATAGGAGAGAATAAAAACGCCTAACAAACTACAAGA | 17 |
| 559 | ACTACGCGGAGTGCGAGACGTCAGGCA | 17 |
| 560 | AGTGAGGACCTGAAACAATGCAAGAATGGCGA | 17 |
| 561 | AGTGGACGCGGTAGCGGGATAAGCAAA | 17 |
| 562 | TACACCGGTAGATATCATAGGAAGGTCACGCAAA | 17 |
| 563 | TGGAGGAGTCAAGAAACTGGCCAAGTGA | 17 |
| 564 | AGCCCTCGGCAGATACGCAAAGTACGACAA | 17 |
| 565 | ATAAGAGGCTCAGAAGATCCAGACGAGTGCAGGA | 17 |
| 566 | ATAAGACAATCAAGAGAATGAACGCATCGGAACACT | 17 |
| 567 | AGGCAGCAGTGGGACCGGTAAAAGCAT | 17 |
| 568 | AGCTAGCTCGGGCGATGGAGGCACGT | 17 |
| 569 | ACAAAGGTGACAAAAGTAACGGGAATACCCACCGT | 17 |
| 570 | ACCAGGATGACCAGGGATCGCGAAGATAGCGGA | 17 |
| 571 | ATCGAGCCCTCAGGAGCTAGGCCAGCA | 17 |
| 572 | TAGAGACGTCACGAGATGAAGGGATAAAGGAAGTCA | 17 |
| 573 | AAGAATGGGAGAACTCCGGACGTACGAGGCT | 17 |
| 574 | ACAGAGGTGAAAAGATAAAGCAGGGTGAGGAAGGCATGC | 17 |
| 575 | AGACAAGAGATAGAAAGCAGTGAAAGAATAGGACGGTC | 18 |
| 576 | AGAGGATGGAGGGCCTCCGGGCGTGGC | 18 |
| 577 | ACGACTAGGACGATGCGGAAATAACGACA | 18 |
| 578 | AGTGGAAACCTAGCCAGCTAAGGAAGCTA | 18 |
| 579 | AGGGCAATGGCAAAGTAAGGAAGTACGGAAA | 18 |
| 580 | TAGGACCATAGAAGACTGGACCGATACAGCGCT | 18 |
| 581 | AGGGCGGGTAAACGAGTGAAAGGGTGGA | 18 |
| 582 | ACAATAAGGACAGTGCAGCAGGTAAGACCACTA | 18 |
| 583 | AAAGACTCCACGACGTACAGAGACTCCGCGCC | 18 |
| 584 | TGGAACGATCGAAGCGTAACGGGCAT | 18 |
| 585 | AAGGAAAATAGAGAAGGTCGAGGAAATAAAGGGAAA | 18 |
| 586 | TGGAGAAACTAAGCGGATAGGGAAATAAACGAACC | 18 |
| 587 | TCAGGGAATCCCAAAGTCCGACCAATGAC | 18 |
| 588 | AGACTGCACGCATCCGAGCGTAAAAACA | 18 |
| 589 | TGAGACCAATAACGAGATCGGCAAGTCGAGA | 18 |
| 590 | AGTCGCAGAATCAAACAACCTGAGAACCTGCGGGAGTGA | 18 |
| 591 | ACGGATAAGACGGTAAGAGAAATAAGAGCATGAGAA | 18 |
| 592 | ACTGGGACGATAGAACGATAGCCAAGTAAAAGGGTA | 18 |
| 593 | AGAGAATGGAACGAAGTGCAAAAAGTGGCAGAA | 18 |
| 594 | TGAACAGATAGGCAGATCAGAAGAATGAGGAAGTCGCAGA | 18 |
| 595 | AATAAGAGGGTGGGAGCGATGCCGGGATGCGCGA | 18 |
| 596 | ATGCGAAGGTAAGAGAATGCAGGAGTAAAGAGGACTGAA | 18 |
| 597 | AAGATCGGGACATGAAACGATAGGAAGGTACGGCGA | 18 |
| 598 | TGAGACAGTACAAAAGTGAAAGGGTGACAGCCTGCGCGGA | 18 |
| 599 | TATCAGGGATGCCACGATGGGCACATGCCCAAAATGAAA | 18 |
| 600 | AAATCACCAAATACCAAAATGAAGCCGATGCGGGA | 18 |
| 601 | ATGCGCTAGCTAACGAGCATAAAACGGTAGGAAA | 18 |
| 602 | ATGGAAAGCTAACCGCAGTGGAAGAATAAGGAGCT | 18 |
| 603 | ACGCAAATACGCCGAATAAGGAAGTAGCGGACT | 18 |
| 604 | AAGGAGGTAGACGAATAGGCGAATAACGCGAGTCGAGA | 18 |
| 605 | AATCCAAGACTACAGGACTCAGCAGATGAAAAAACTAGAA | 18 |
| 606 | ACGTGAAGGCCTAAGAAACATAAGACACTGAAAGAGTAGCGGAGGCATGC | 18 |
| 607 | AGAGAGTAAGGAAATGAGAACAGTGAAGACATCCCAAGA | 19 |
| 608 | AATGAAAAAAGTGGAGAGGTCGAACGGTAGAGCAG | 19 |
| 609 | TGGAGAAGGATACGCCGATCGCCGGGA | 19 |
| 610 | TAACCGGGCTAAACACAATGAAACACGTGGCCGA | 19 |
| 611 | ATACGGAGGAATCAGAGGAGGTGGCAGGAC | 19 |
| 612 | TGAACGAGTGGGCGGGATAGAAAAACTACAGCGA | 19 |
| 613 | TACGCGCGATAGGAACCTACGAGAACTAAGAGGA | 19 |
| 614 | TAAAGACATAAGGGCCTACGCACGAGTAAAAGAGT | 19 |
| 615 | ACCGACGTCAGACAATAGAAGGGTAAAAAGATGAACCGA | 19 |
| 616 | TGAGCAAAATCCAGGCGTCGCAAGGTC | 19 |
| 617 | ACGCAGTCAAGACATAAGAGAATGCCAGAAGTACA | 19 |
| 618 | AGCCTGGGACGGCTGAGAGAGATCGGG | 19 |
| 619 | CAGTCAAAAGGGTCAGGACATAGCGGGAT | 19 |
| 620 | AGCCGAATGCAAAGATACGACGGTGCAAGAA | 19 |
| 621 | TCACGGCATAGGCAAGTGCAAAACGTAACAAC | 19 |
| 622 | ACTGGCCAAAATGGAAGACTGAACGCATGAC | 19 |
| 623 | ACGGGTCACGCAGTGCAGACCTGCA | 19 |
| 624 | ACAGTACAGAAATGGAAAACTAGAAGAGTAAGCAAATCGAA | 19 |
| 625 | ACCTCCAAGGGTGGAAGGATGGACAGGTGA | 19 |
| 626 | ACAGGTAAAGAGATCGCGGACATGAGAAGGT | 19 |
| 627 | ACAAAGCTAAACAAGTCGGGAGGTGAACGAATA | 19 |
| 628 | AGGACGGGTAAGGGACCTGGACCGGA | 19 |
| 629 | ATGGCAACATGCAAACATAAGAGGGTCAACCAA | 19 |
| 630 | TGGAAGGCTGAAAAGATCGAAAAATGGGCGAATACAA | 19 |
| 631 | AAGGTAAAGGGATAGCGGGATCAGAAGGTGGGACGA | 19 |
| 632 | TGAAAGAATGAAGAAATACCAAGCGATAACACGATCCGGA | 19 |
| 633 | ACTGCGGGAGCTGAACAAGTCACCGCT | 19 |
| 634 | AGCCAGAGGGTGCAGGGGATATCAAA | 19 |
| 635 | TGGGCAGATACGGAGCGATAAAAACATGAAAGG | 19 |
| 636 | AGTGGGCCACTGGAAGGATCAGCACGTA | 19 |
| 637 | ACGGCCTAAAGGACTAAGAGCACATGAGCGA | 19 |
| 638 | AGTGAAGAGAGTGAAGAAACTAAGAAAGAGTAGAGAGATGCG | 19 |
| 639 | AGAGATAGCGAAAATCGACAACATCGCGGGAG | 19 |
| 640 | TGGAAAACTGACGGGATGACGAGAAGCATGC | 19 |
| 641 | AGAACAGACTAAAAGAATCAACAGATAGAGGAATGAGGAAGTGC | 20 |
| 642 | AGGAAGTAAGGAAAACTGGAAGAGTAACACAATGGGAGA | 20 |
| 643 | ATACAAAAGTCAACCAGATGGACAGATAGAGAAATGACGAGA | 20 |
| 644 | TGGAAACAGTCACACGCTAAGGGAATGGACGCG | 20 |
| 645 | TGACCAGATCGGGAAGATCCGGGCAAT | 20 |
| 646 | AGGACAGTAGAAAGGTGCAGGAATGACAAGATGGCCA | 20 |
| 647 | AATCACAGCATAGAGCCAATAAGACGGTAAAAGGCGT | 20 |
| 648 | AGCACGATAGGACGGGTCACGAGAGTGAG | 20 |
| 649 | AGAGGTGCCAACAACTAGGACAATAAGCCGATAAA | 20 |
| 650 | AGCGTCGAACAATAGAGACGTCCAGAGAATGGACCCA | 20 |
| 651 | ATCCACCGGATAGCAAGAGTAGCGGAGATAAGA | 20 |
| 652 | ACGTCAGGAGATAGCGAAGTCACGAAATGAGAGAGTC | 20 |
| 653 | ACGCGGTAGCACAATAAAGACGTACAAAAGTACAACA | 20 |
| 654 | AAGTGAGAACATCAAAACGATAAGCAGGATAAAAAGGTAAA | 20 |
| 655 | ACGGGATCGGGACGCTCGAAAACTGACGA | 20 |
| 656 | ACTAGAAAAGTAAGAACCTAGAAAAATAGCGGCATAGAAAACT | 20 |
| 657 | AAGGGAATGGCGAACATAAGAGGAATAGGAAGGTGGCGA | 20 |
| 658 | AGTGGAGCAATAAAGGAGGTGGGACGGTCA | 20 |
| 659 | AGAGCTAGAGAAATCGCAACGATACCGGAATCGGGA | 20 |
| 660 | AGTAGAACAAATCAGCGGCGTAGGACAAGT | 20 |
| 661 | CCGGGAATCCACGAGTCGAAAAAATAAGACACTC | 20 |
| 662 | AGAGAGTGCGGAGGCTAAACGGGTGGAA | 20 |
| 663 | AGAACTGGAAAGATCCAGAGCATCGCAGAAT | 20 |
| 664 | AAGACGAGTGCGCGGATCAACGGATA | 20 |
| 665 | ACCACCTAAGGGCGTCGGGCGATA | 20 |
| 666 | ACGGCAGTACCGAAATGGGCTAGCC | 20 |
| 667 | ACGGGATGGGAGAATGGAACCGTAGGA | 20 |
| 668 | CCGGTAGAGAAAGTGGAACACTACGGAGAATGCA | 20 |
| 669 | ACAGTAGGAAAGCTGACGGGCTAAGGCCGGT | 20 |
| 670 | AGGACAAGCTCCGCGCGTGAAGATA | 20 |
| 671 | TCGCGGAGTAGAGGAATACCGGGCC | 20 |
| 672 | ATGACGGGCACTCAACGGCTGAAAG | 20 |
| 673 | AGCTGAAGCAGGTGCAGGACTGGG | 20 |
| 674 | AGGAGTAGGGACGATCACAGGAGCATGC | 20 |
| 675 | AGAAGAAAAGTGGAACACTAGGCGGGTGAACGGGA | 21 |
| 676 | TGAAAAGGATAGAGCGGTGCGAAAGTCAAGAAA | 21 |
| 677 | TAGGAGAACTAACGAAGTGGGAGAGACTAAAGACGTCAGA | 21 |
| 678 | AGATCAAAAGGTCGGAGGCAATGGAGAAGTGCAGCA | 21 |
| 679 | ATCAGACCGTCCGGCGCTGAAAACCA | 21 |
| 680 | ATGAAGACAGATCCAAGAGCTGAGCAGCTAGCGA | 21 |
| 681 | AGAGTGGCGAGCGTAAAGCAGTAGGGAGGTAA | 21 |
| 682 | AAGAACTACAGGCCTCAGACAATCCAGACGTA | 21 |
| 683 | AGAGGGATACAGCCGTCAGGGAGGTA | 21 |
| 684 | AGAGAATGAAAGGATGCGCCCATGAGCCA | 21 |
| 685 | ACTGGGAGGGCTAAAAAACTAGAAGAGTGAAAA | 21 |
| 686 | ACTGGCACCGTGAGAAAATAGCACAATACGAA | 21 |
| 687 | AGGTCGAGAAATCGGGAAAGTGCGGA | 21 |
| 688 | AGTGGACCACTAGCGAGATCAACAGAGTAGGGACA | 21 |
| 689 | TCGAAGAATAAGGCAGAATGCGACAGTACGGGAGA | 21 |
| 690 | TCGAAAGACTGCGAGCGTGACGAGATGA | 21 |
| 691 | AGAGGTAAAAAACTGAAAACATGAGGCGGTGAAAGGGA | 21 |
| 692 | ATAACCAGGTGGGACACAGTGACGGGCA | 21 |
| 693 | TGGCGGGCCTCAAGAGATGCACGACTGAGC | 21 |
| 694 | AGGTGAGAAGGTACACAGATACGAGAATGGAACGGCTCAA | 21 |
| 695 | AACAATAAGAAGGTCGGCCCGTGAGACCAGGTA | 21 |
| 696 | AGAGAGCTGGAGGACCCGCGGAGGTG | 21 |
| 697 | AGCGGGTCAAAAAATCGAGAGATAAGGAGAGTGA | 21 |
| 698 | ACGGGTGAAGACAGTAGAAAAATGAGAGAAATCCGGCAA | 21 |
| 699 | ATAGCAGGACTGGACGCGTACGAAAGAGTGGCAA | 21 |
| 700 | AATAAGCGGATGGCGAGATGGCGGGC | 21 |
| 701 | TCGGCGAGATAGCAAGATGAAGACGTAAGAACGGTAA | 21 |
| 702 | AAGGCTCAAGAGATGAACAAATAAAGAGATACGCGGCTAA | 21 |
| 703 | AAGACCTAAAAGGATAAGAAACTCAAGGCAGTGACGA | 21 |
| 704 | AACGTGCAAGCAGTAACAGAATGAGAAAGGATGA | 21 |
| 705 | ACACCGTGGCGGGATAGGAGAGTGGAGAAATGGAAGAAT | 21 |
| 706 | ACAAGAATAAGAACGGTCGGACGCATAAACAGGTAAGCCA | 21 |
| 707 | ATAAGAGAGGTCAGCACACGTACGAAGGAAGATCT | 21 |
| 708 | AGACGGATAGAAGCATGGCAGAGGATCAGGGA | 22 |
| 709 | AGTCAAACGAATACAAAGATAGCCAACTAGGACCAA | 22 |
| 710 | TCAGAGACTGAGAAGCGTAAGCGAAGTACGACA | 22 |
| 711 | ATCGGAAAGTCGAGGGATGCGAGAGATACAAA | 22 |
| 712 | AGTAAGAGGGTAGAAGGCAGTCGGGAGCCA | 22 |
| 713 | TCCGGGAGCTAACAAAATAAAGAACTGGCAGAGGCT | 22 |
| 714 | AGCGCTCCGGGAGATAGGAAGGATGAC | 22 |
| 715 | AGGCCGTCCGAAAGTACGGAAATCAAAAAGTG | 22 |
| 716 | AGGCACTCAGAGAGTGAAAGCGTAAGAACGGGT | 22 |
| 717 | AGGGAGCTAGGCGGGTAGGCCACA | 22 |
| 718 | TCACGGGATAAAGAGATGACAAGCGTGAAGGA | 22 |
| 719 | ATCCACGGAGTGCGCAGACGTCCAA | 22 |
| 720 | AGGTCCACAACTCCGCCGGGTACA | 22 |
| 721 | ACGGTGAAGCAAATCACGGGCTCGAA | 22 |
| 722 | AAGGTGGCGAGATGGGACCATGAAAGAATCGA | 22 |
| 723 | AGGGTAAGACGATCCAGAGATGAGCCCATAACAAGGT | 22 |
| 724 | AACAACCTAACAGAAGTACCAGAATAGAGCAACTGAAAAAGT | 22 |
| 725 | ACAGCACTGGCAACGTCCAAGGCTCGCGGCC | 22 |
| 726 | TGCGCGCGTGCGCCGAAATAAGGACAAT | 22 |
| 727 | AACGACCTAGGACCGAATACAAAAGCTAACAGACTC | 22 |
| 728 | AGAGCATCCAACGCTGAGCCAGTCAGAAGGT | 22 |
| 729 | AGAGAAGTAAGCGAAATAAAGAAAATAAAGAAATGCCGCGA | 22 |
| 730 | TCGGAAGGTGAAGAACTACCAGGCTACGGAGAA | 22 |
| 731 | TGAAAGGGTAAGAGGGTAAGGACATACAAGAATAAAGAAGCT | 22 |
| 732 | AGGACAATAAACGCCCTAAAACCGCGGAGAGAA | 22 |
| 733 | TAGCCCGATACAAGCGTCCGGGCAT | 22 |
| 734 | AGAAACCTGGAGAAATAGGACAGGTGAAAGGCTGGGC | 22 |
| 735 | ACATGACCGACTGGAGAGCATGGAAACATACACCC | 22 |
| 736 | AGTCAAAAAGTCGAGGAATAGCCGGGTGGC | 22 |
| 737 | ACGGTAACAAAATCAAGAAAATACCGGAGGGTGCAGA | 22 |
| 738 | AGGGTACGACCGGTACAGGACCTAAGAACGA | 22 |
| 739 | TGGCGAGAGTCGGGCCGTGAGGACAGTAAGG | 22 |
| 740 | ACGTGGAGGAAGTAGCAGAATAGCGGGATAGCCAGCT | 22 |
| 741 | ACGAAGAAATGGGCAAGTGCGGAGAAGATCT | 22 |
| 742 | AGATCGAAAAATAGGGAGGTGGCCGGCTGCGA | 23 |
| 743 | AAGTCGGGCGGGTGAAAGCAACTAAAAGGA | 23 |
| 744 | TCGAAGCATGAAAGAGTAGGAAAGTGGAAGAATGAGA | 23 |
| 745 | AGATAACGAAATAAGGAAGGTAAAACGGTGGAGAGATAGAGGACA | 23 |
| 746 | TAACAAAGTGGAAACACTCAAGAGCTAAGGGAACTAGA | 23 |
| 747 | AGCATAACGGAATGGCTAGCATCGGGAGAGT | 23 |
| 748 | AGCGGCATGAAGCGATAGGGAACGCTGACA | 23 |
| 749 | AGAAATAACCGAATCGGGAAATCAAACCATAGAAGACT | 23 |
| 750 | AGACGGGTAACAGAATGGAGGCAATAGGAAACGT | 23 |
| 751 | AAGACACTAACGGGATACCACGAGTGACAAGA | 23 |
| 752 | TCGGAGGATGGCACCATGAAAAGATAGAGAGCT | 23 |
| 753 | AGAAGGGTACGGGAATAGAAAAATGCAAAGCTAGGGC | 23 |
| 754 | CGTCGACCGGTGAGAAGGGTAAAAAGGGTGACAA | 23 |
| 755 | AATGAGAGAATAACCAGATAGGGACGTGAAAGGCT | 23 |
| 756 | AGGCACCTGGAGACAATGAGGCAGTACACGCGT | 23 |
| 757 | ACCAAGATCCAGAGAATCAGACGGTGAGACACTGGAC | 23 |
| 758 | ACCATAAGAAGATGAGGAGGTGAGGGACATGAAACA | 23 |
| 759 | ATAACAACAATAACCACATAAGGGCCTCGAAACGTGG | 23 |
| 760 | AGAGCATACAGCCGGTGCAAAAGTGAGACGGA | 23 |
| 761 | TGCGAAAAATGAACAGGCTGGGACGATACCC | 23 |
| 762 | AGAATGCCAAGATGGCGGCCTGCCGGGA | 23 |
| 763 | TCACCGAGTAGCCAACCTGACAAAAAGTGCGGAA | 23 |
| 764 | ATACGGGCATGAGAAGGTGGAGCACTCAG | 23 |
| 765 | ACGATGAAGACGATACGGACGTACCAAAATGGAA | 23 |
| 766 | AACAATGGGAGCGTAAGCAAAATCAGACGGTAGA | 23 |
| 767 | ACGGTAAAGAGATGCACAAGATGAAGAGCATCA | 23 |
| 768 | ACACATGACAGCCCGCGGGAGTAGG | 23 |
| 769 | CGGAGTAAGCGGGTAACGAGGTGAGCACATA | 23 |
| 770 | AAAAGGTCGCAAAGTACGAAGGTAAGGGAGTGGAG | 23 |
| 771 | AGAGTGAAGGGCCTGGAGGGAGTCGGA | 23 |
| 772 | ACAATGCCGACGTAAGCAAGTCAAACGCTAAGGCA | 23 |
| 773 | AGATAACGGAATGCGAAACTGGCGCCCT | 23 |
| 774 | AAGAACGTAGGACCATAACAAGGTAGAAAAAAGATCT | 23 |
| 775 | AGATGGAAAGCTGAGAGAAAGTCAGAAGATCACAGAC | 24 |
| 776 | TCAGGAGGATCGGGCAGTAGACACGTAAGA | 24 |
| 777 | AGGTAGAGGCCAATGACAGGCTGAAGAGGTGA | 24 |
| 778 | AGGCCTAAAAGAATACGCGGGTCAGGAACA | 24 |
| 779 | ATGCGAAGCCTGGGACGATCGGGAGATA | 24 |
| 780 | AAAGGGATAGAGAAATGGCCGAATAGACCGGT | 24 |
| 781 | ACGGCGCGCTAGCGATAAGGAACT | 24 |
| 782 | AGACGGGTAAAACCGTAGGAAGCTCAGAAACA | 24 |
| 783 | TAACGAAAGCTACAGCAGGTAAGCAAGCTAAGAACC | 24 |
| 784 | TGAGCAGGATAACGCAGTAAGGACACTACGGGA | 24 |
| 785 | ACTAAGCCAGATGACCGGGTACGAACGTCAA | 24 |
| 786 | ACGAATAGCAAAGGTGCGGGACGTGGC | 24 |
| 787 | CGAAGATAGAAAACATACCCAAATGCCGGAATGGGAGA | 24 |
| 788 | ATGCGCAAGTAAAAACATAGGAACAGTAAGGCAA | 24 |
| 789 | TCGGGAGGTGAAAGGGAATGAGACAACTAACA | 24 |
| 790 | AGGTGGGAAAAATCCAGCAGTAAACAGCATAGGGCA | 24 |
| 791 | ATGAGAAGGTAGAACAATAAAGACGATGAGGAAGTAAAAACGGG | 24 |
| 792 | TGGGCAAATGACACGATGAAAAAGTAACGGAGTAA | 24 |
| 793 | ACCCACTCAGAAACTGGAAAAAGTCGAAACATGGGA | 24 |
| 794 | AGAATACACCACATCCAGCGAATAACGCGACTCCCA | 24 |
| 795 | ACCAATAGACGAGTGAAGAGATGGAAGCCCTGGCGA | 24 |
| 796 | ACATGGAGACAATAGGAGGGTCAAGGACGTGGAC | 24 |
| 797 | ACGTACAGCGGTAACGGCCTCAGCAGG | 24 |
| 798 | TGGGAGGCTACGACGAATGGAGGAGTGC | 24 |
| 799 | AGCAATAGGCGGGATAGCAGCCTGCA | 24 |
| 800 | AGGATCGGCAACTGAGAAAGTGAAGAGAATGCCA | 24 |
| 801 | ACCCTGCAAGCGTAAAAAGCGTGAAGGCG | 24 |
| 802 | TCCAGAAGTACGCACACTGGACAAATAGACGAAT | 24 |
| 803 | AAGGGCCTCAAAAGCATCGCGAGGAT | 24 |
| 804 | AAGAAAGTGCGGGAGTAACAACCTCGGGA | 24 |
| 805 | AGTCGGAAAGTAGAAAGGTGGACCGATAACCCG | 24 |
| 806 | CGGGCACAATAAACGAACTGCGAGCA | 24 |
| 807 | TCAGAGAGGTGCAGAAGATACCACGAGTAGAGGCA | 24 |
| 808 | TGGGAACGTGAAAACAATAACGCAAGTCAGGACGAGAGATCT | 24 |

TABLE 2
Backbone sequences used in the eight sample multi-plex assay of Example 1

| | Underlying | | Underlying Spot | | Underlying Spot |
|---|---|---|---|---|---|
| DV2 Tag # | Spot Sequence | DV2 Tag # | Sequence | DV2 Tag # | Sequence |
| tag-306 | 3-5-10-16-17-22 | tag-466 | 3-5-12-14-19-24 | tag-249 | 2-8-10-13-18-24 |
| tag-488 | 4-6-11-13-18-24 | tag-498 | 2-7-12-13-18-24 | tag-517 | 1-6-9-15-20-22 |
| tag-015 | 3-6-12-13-18-24 | tag-565 | 1-6-11-16-17-22 | tag-535 | 4-5-12-14-19-24 |
| tag-025 | 3-8-9-15-20-22 | tag-584 | 4-6-9-15-20-22 | tag-588 | 3-8-11-13-18-24 |
| tag-192 | 3-5-12-15-20-22 | tag-814 | 1-7-10-16-17-22 | tag-599 | 3-6-11-16-17-22 |
| tag-198 | 1-8-10-15-20-22 | tag-134 | 4-6-9-14-19-24 | tag-627 | 1-7-9-16-17-22 |
| tag-265 | 4-7-9-14-19-24 | tag-143 | 1-6-12-13-18-24 | tag-662 | 1-8-9-14-19-24 |
| tag-403 | 4-5-10-16-17-22 | tag-205 | 4-7-10-13-18-24 | tag-764 | 4-6-12-15-20-22 |
| tag-529 | 1-6-11-13-18-24 | tag-270 | 1-8-11-14-19-24 | tag-046 | 2-7-12-15-20-22 |
| tag-596 | 4-6-9-16-17-22 | tag-317 | 2-5-12-15-20-22 | tag-109 | 2-5-11-13-18-24 |
| tag-678 | 2-7-12-14-19-24 | tag-340 | 2-5-10-16-17-22 | tag-200 | 3-8-9-16-17-22 |
| tag-700 | 2-5-11-16-17-22 | tag-503 | 3-6-9-16-17-22 | tag-217 | 4-7-9-15-20-22 |
| tag-725 | 2-8-11-14-19-24 | tag-600 | 3-8-10-15-20-22 | tag-254 | 3-5-12-13-18-24 |
| tag-759 | 1-7-10-13-18-24 | tag-737 | 2-8-9-15-20-22 | tag-331 | 1-8-10-16-17-22 |
| tag-101 | 3-8-10-13-18-24 | tag-815 | 4-5-11-16-17-22 | tag-344 | 4-6-12-14-19-24 |
| tag-195 | 3-6-9-15-20-22 | tag-816 | 1-7-12-14-19-24 | tag-773 | 2-8-11-16-17-22 |
| tag-236 | 2-5-12-14-19-24 | tag-919 | 2-7-9-14-19-24 | tag-802 | 4-5-10-13-18-24 |
| tag-271 | 1-7-12-13-18-24 | tag-013 | 4-6-11-14-19-24 | tag-803 | 3-6-12-14-19-24 |
| tag-470 | 1-8-11-13-18-24 | tag-039 | 2-8-9-16-17-22 | tag-805 | 1-6-11-14-19-24 |
| tag-493 | 1-6-12-15-20-22 | tag-154 | 3-8-10-16-17-22 | tag-924 | 1-7-10-15-20-22 |
| tag-512 | 4-6-11-16-17-22 | tag-158 | 1-6-12-14-19-24 | tag-004 | 3-8-11-14-19-24 |
| tag-528 | 4-7-10-16-17-22 | tag-259 | 1-7-12-15-20-22 | tag-047 | 3-5-11-16-17-22 |
| tag-607 | 4-5-11-14-19-24 | tag-336 | 4-5-11-13-18-24 | tag-066 | 3-6-11-13-18-24 |
| tag-629 | 2-8-9-14-19-24 | tag-402 | 2-7-9-15-20-22 | tag-130 | 2-8-10-15-20-22 |
| tag-790 | 3-5-10-15-20-22 | tag-418 | 3-5-10-13-18-24 | tag-392 | 4-6-12-13-18-24 |
| tag-809 | 2-7-9-16-17-22 | tag-665 | 2-5-12-13-18-24 | tag-463 | 4-5-12-15-20-22 |
| tag-026 | 1-8-10-13-18-24 | tag-731 | 3-6-9-14-19-24 | tag-686 | 1-8-9-15-20-22 |
| tag-041 | 4-7-9-16-17-22 | tag-741 | 4-7-10-15-20-22 | tag-689 | 2-8-10-16-17-22 |
| tag-075 | 3-8-9-14-19-24 | tag-911 | 1-8-11-16-17-22 | tag-693 | 4-7-12-14-19-24 |
| tag-219 | 3-6-12-15-20-22 | tag-002 | 4-7-12-13-18-24 | tag-750 | 2-7-10-13-18-24 |
| tag-225 | 2-8-11-13-18-24 | tag-006 | 2-5-10-15-20-22 | tag-804 | 1-7-9-14-19-24 |
| tag-260 | 2-5-11-14-19-24 | tag-056 | 2-7-10-16-17-22 | tag-905 | 1-6-9-16-17-22 |
| tag-427 | 4-5-10-15-20-22 | tag-218 | 3-5-11-14-19-24 | | |

TABLE 3
Dye colors coupled to oligos for each sample for
the eight sample multi-plex assay of Example 1.

| | | SAMPLE ID | | | | | | | |
|---|---|---|---|---|---|---|---|---|---|
| | | A | B | C | D | E | F | G | H |
| SPOT | 1 | Blue | Blue | Blue | Blue | Blue | Blue | Blue | Blue |
| ID | 2 | Green | Green | Green | Green | Green | Green | Green | Green |
| | 3 | Yellow | Yellow | Yellow | Yellow | Yellow | Yellow | Yellow | Yellow |
| | 4 | Red | Red | Red | Red | Red | Red | Red | Red |
| | 5 | Blue | Blue | Blue | Blue | Blue | Blue | Blue | Blue |
| | 6 | Green | Green | Green | Green | Green | Green | Green | Green |
| | 7 | Yellow | Yellow | Yellow | Yellow | Yellow | Yellow | Yellow | Yellow |
| | 8 | Red | Red | Red | Red | Red | Red | Red | Red |
| | 9 | Blue | Blue | Blue | Blue | Blue | Blue | Blue | Blue |
| | 10 | Green | Green | Green | Green | Green | Green | Green | Green |
| | 11 | Yellow | Yellow | Yellow | Yellow | Yellow | Yellow | Yellow | Yellow |
| | 12 | Red | Red | Red | Red | Red | Red | Red | Red |
| | 13 | Blue | Blue | Blue | Blue | Blue | Blue | Blue | Blue |
| | 14 | Green | Green | Green | Green | Green | Green | Green | Green |
| | 15 | Yellow | Yellow | Yellow | Yellow | Yellow | Yellow | Yellow | Yellow |
| | 16 | Red | Red | Red | Red | Red | Red | Red | Red |
| | 17 | Green | Blue | Blue | Blue | Yellow | Yellow | Green | Green |
| | 18 | Yellow | Yellow | Yellow | Red | Red | Red | Green | Green |
| | 19 | Yellow | Yellow | Yellow | Red | Red | Red | Blue | Blue |
| | 20 | Green | Blue | Blue | Blue | Red | Red | Green | Green |
| | 22 | Blue | Green | Red | Yellow | Blue | Green | Yellow | Red |
| | 24 | Blue | Green | Red | Yellow | Blue | Green | Yellow | Red |

Dye colors coupled to oligonucleotide for each sample for eight sample-plex assay (Blue = Alexa 488, Green = Alexa 546, Yellow = Texas Red-X, Red = Alexa 647). Spots ID numbers 1 to 16 were used to identify the target nucleic acid and Spot ID numbers 17 to 20, 22, and 24 were used to identify the sample.

To clarify Tables 2 and 3, DV2 tag-306, as an example, which has an underlying spot sequence of 3-5-10-16-17-22, would comprise (in order) a first position hybridized to a plurality of yellow fluorophore labeled oligonucleotides, a second position hybridized to a plurality of blue fluorophore labeled oligonucleotides, a third position hybridized to a plurality of green fluorophore labeled oligonucleotides, and a fourth position hybridized to a plurality of red fluorophore labeled oligonucleotides; the first through fourth positions are for identifying a target nucleic acid. The DV2 tag-306 would identify the sample as Sample A if it further comprises (in order) a fifth position hybridized to a plurality of a green fluorophore labeled oligonucleotides followed by a sixth position hybridized to a plurality of a blue fluorophore labeled oligonucleotides. However, the DV2 tag-306 would identify the sample as Sample B if it instead further comprises fifth and six positions that are hybridized to a plurality of blue fluorophore labeled oligonucleotides and green fluorophore labeled oligonucleotides.

Spot sequences/Spot IDs 1, 5, 9, 13, 17, and 21 correspond to SEQ ID NO: 1 to SEQ ID NO: 33, SEQ ID NO: 133 to SEQ ID NO: 166, SEQ ID NO: 268 to SEQ ID NO: 302, SEQ ID NO: 405 to SEQ ID NO: 437, SEQ ID NO: 542 to SEQ ID NO: 574, and SEQ ID NO: 675 to SEQ ID NO: 707, respectively.

Spot sequences/Spot IDs 2, 6, 10, 14, 18, and 22 correspond to SEQ ID NO: 34 to SEQ ID NO: 66, SEQ ID NO: 167 to SEQ ID NO: 200, SEQ ID NO: 303 to SEQ ID NO: 336, SEQ ID NO: 438 to SEQ ID NO: 473, SEQ ID NO: 575 to SEQ ID NO: 606, and SEQ ID NO: 708 to SEQ ID NO: 741 respectively.

Spot sequences/Spot IDs 4, 8, 12, 16, 20, and 24 correspond to SEQ ID NO: 101 to SEQ ID NO: 132, SEQ ID NO: 234 to SEQ ID NO: 267, SEQ ID NO: 371 to SEQ ID NO: 404, SEQ ID NO: 508 to SEQ ID NO: 541, SEQ ID NO: 641 to SEQ ID NO: 674, and SEQ ID NO: 775 to SEQ ID NO: 808, respectively.

Spot sequences/Spot IDs 3, 7, 11, 15, 19, and 23 correspond to SEQ ID NO: 67 to SEQ ID NO: 100, SEQ ID NO: 201 to SEQ ID NO: 233, SEQ ID NO: 337 to SEQ ID NO: 370, SEQ ID NO: 474 to SEQ ID NO: 507, SEQ ID NO: 607 to SEQ ID NO: 640, and SEQ ID NO: 742 to SEQ ID NO: 774, respectively.

### Example 2

### Thirty-Two Sample-Plex Assay Using Probes Comprising Three Positions for Target Identification and Three Positions for Sample Identification

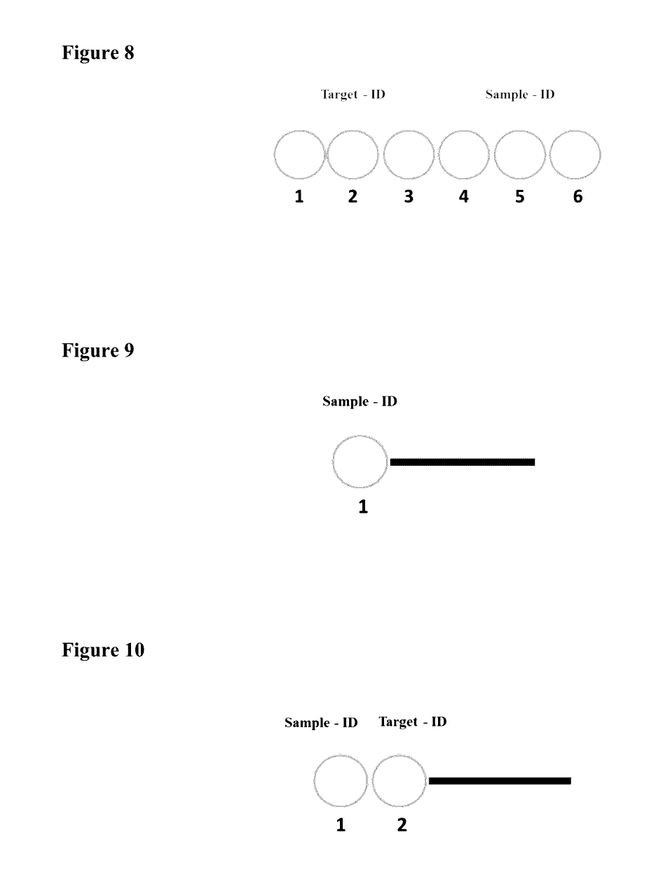

The steps used in Example 2 are similar to those described in Example 1 with the exception that the six position probe backbone used in this Example had three positions for target identification and three positions for sample identification. Here, the first three positions adjacent to the thirty-five deoxynucleotide target binding domain were for target identification. A schematic of a backbone used in this Example is shown in FIG. 8. Backbone sequences and labeled oligos used for each sample are listed in Table 4 and Table 5.

After the hybridization reactions, samples were either processed as single samples (a single-plex assay) or pooled into a combined sample with thirty-one other samples (a thirty-two sample multi-plex assay). Samples were processed on a NanoString Technologies® Prep Station and codes were counted with a Gen2 Digital Analyzer.

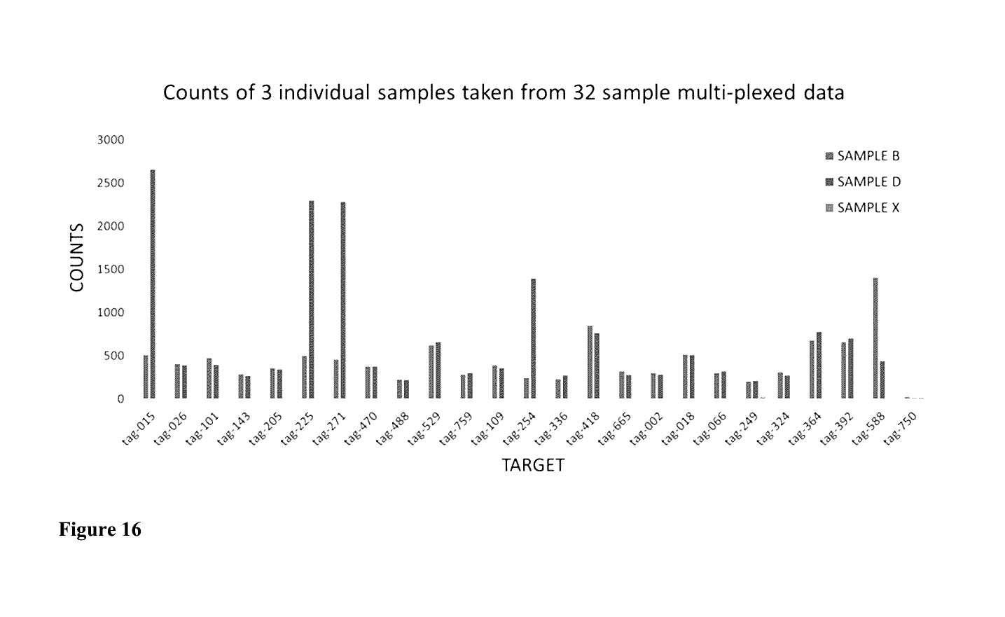

FIG. 16 shows a subset of data from this Example. Here, thirty-two independent hybridization reactions with differing amounts of target nucleic acid and different mixes of labeled oligonucleotides to identify samples were used (see Table 5 for oligo spot colors used for each sample). Each reaction contained probes against twenty-five target nucleic acids and thirty-two samples (totaling 800 total data points). The thirty-two samples had various concentrations of twenty-five target nucleic acids from 320 fM to 3.2 fM. 15 μl of each hybridization reaction was pooled (480 μl total) and 120 μl of this combined sample was loaded onto each of four lanes on a NanoString Technologies® Prep Station. Counts were determined with a NanoString Technologies® Digital Analyzer. Counts were summed across all four lanes for the final counts shown in the Figures. Samples B and D had identical concentrations for twenty of the twenty-five target nucleic acids. Sample B had one target nucleic acid at a higher concentration (orange arrow) and Sample D had four target nucleic acids at a higher concentration (blue arrows). Sample X contained none of the target nucleic acids and gave almost zero counts.

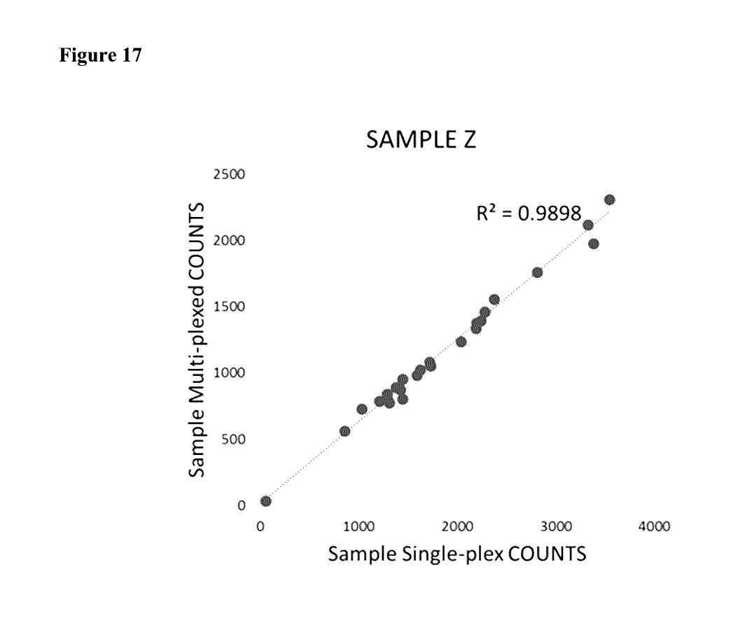

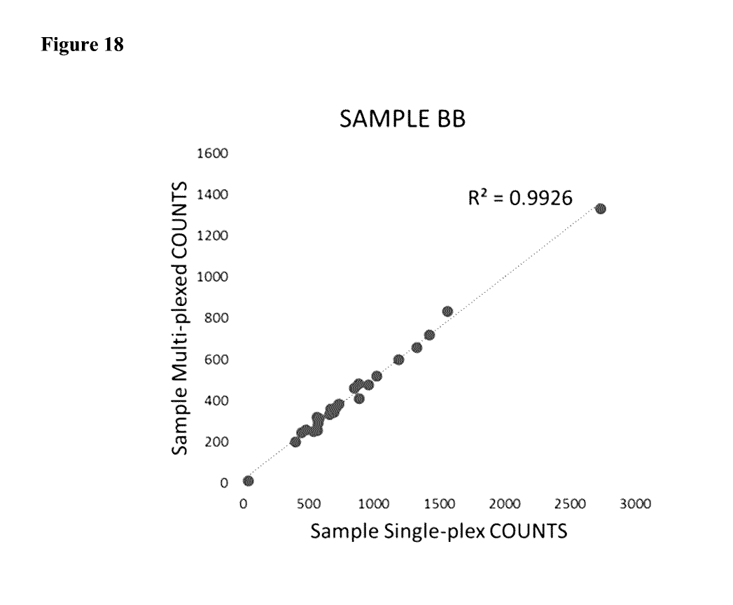

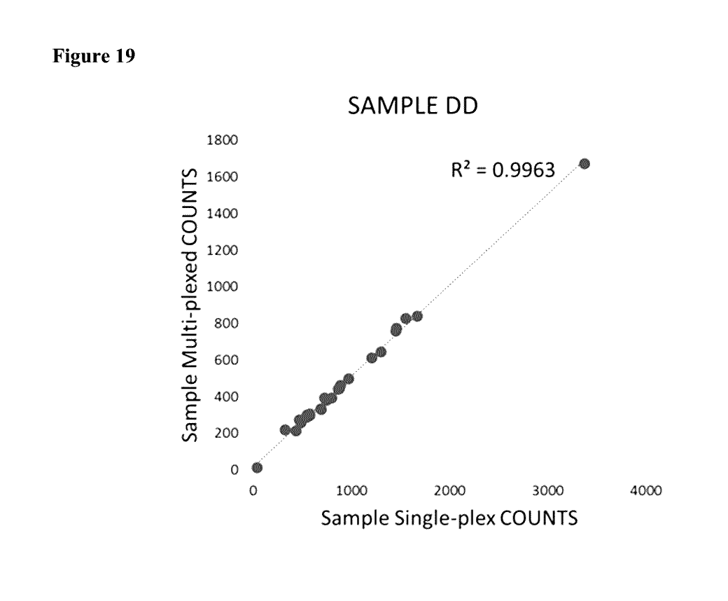

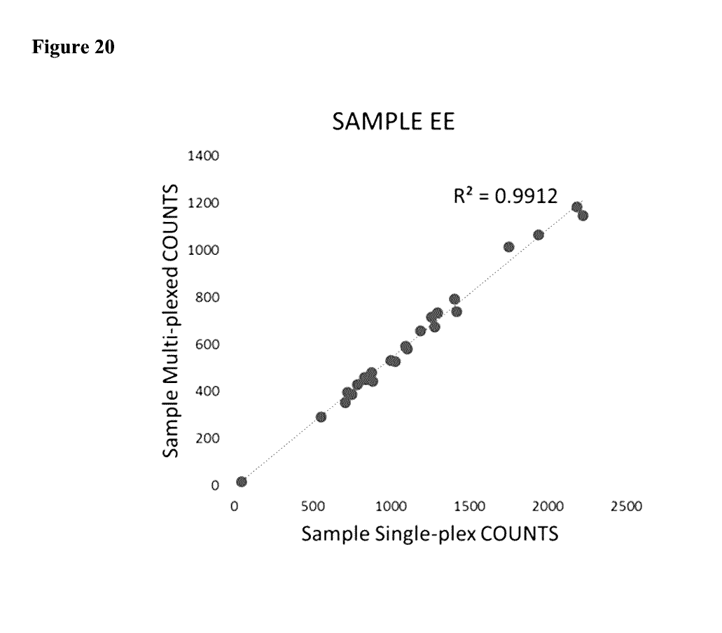

FIGS. 17 to 20 show high correlation (nearly 1.00) between counts from samples detected alone and not pooled into a combined sample (a single-plexed assay) and those samples that were pooled into a combined sample (a multi-plexed assay). Here, plots of counts from hybridization reactions with identical amounts of target nucleic acid processed as a single-plex (one hybridization, not mixed with other hybridzations) or multi-plexed (present with thirty-two total separate hybridization reactions combined).

TABLE 4
Backbone sequences used in the thirty-two sample
multi-plex assay of Example 2

| | Underlying Spot | | Underlying Spot |
|---|---|---|---|
| DV2 Tag # | Sequence | DV2 Tag # | Sequence |
| tag-418 | 3-5-10-13-18-24 | tag-759 | 1-7-10-13-18-24 |
| tag-665 | 2-5-12-13-18-24 | tag-002 | 4-7-12-13-18-24 |
| tag-015 | 3-6-12-13-18-24 | tag-018 | 3-5-11-13-18-24 |
| tag-026 | 1-8-10-13-18-24 | tag-066 | 3-6-11-13-18-24 |
| tag-101 | 3-8-10-13-18-24 | tag-109 | 2-5-11-13-18-24 |
| tag-143 | 1-6-12-13-18-24 | tag-249 | 2-8-10-13-18-24 |
| tag-205 | 4-7-10-13-18-24 | tag-254 | 3-5-12-13-18-24 |
| tag-225 | 2-8-11-13-18-24 | tag-324 | 4-5-12-13-18-24 |
| tag-271 | 1-7-12-13-18-24 | tag-336 | 4-5-11-13-18-24 |
| tag-470 | 1-8-11-13-18-24 | tag-364 | 2-5-10-13-18-24 |
| tag-488 | 4-6-11-13-18-24 | tag-392 | 4-6-12-13-18-24 |
| tag-498 | 2-7-12-13-18-24 | tag-588 | 3-8-11-13-18-24 |
| tag-529 | 1-6-11-13-18-24 | tag-750 | 2-7-10-13-18-24 |

TABLE 5
Dye colors coupled to oligos for each sample for the
thirty-two sample multi-plex assay of Example 2.

| | | SAMPLE ID | | | | | | | | | | | | | | | |
|---|---|---|---|---|---|---|---|---|---|---|---|---|---|---|---|---|---|
| | | A | B | C | D | E | F | G | H | I | J | K | L | M | N | O | P |
| SPOT | 1 | B | B | B | B | B | B | B | B | B | G | G | G | G | G | G | G |
| ID | 2 | G | G | G | G | G | G | G | G | G | Y | Y | Y | Y | Y | Y | Y |
| | 3 | Y | Y | Y | Y | Y | Y | Y | Y | Y | R | R | R | R | R | R | R |
| | 4 | R | R | R | R | R | R | R | R | R | B | B | B | B | B | B | B |
| | 5 | B | B | B | B | B | B | B | B | B | G | G | G | G | G | G | G |
| | 6 | G | G | G | G | G | G | G | G | G | Y | Y | Y | Y | Y | Y | Y |
| | 7 | Y | Y | Y | Y | Y | Y | Y | Y | Y | R | R | R | R | R | R | R |
| | 8 | R | R | R | R | R | R | R | R | R | B | B | B | B | B | B | B |
| | 10 | G | G | G | G | G | G | G | G | G | Y | Y | Y | Y | Y | Y | Y |
| | 11 | Y | Y | Y | Y | Y | Y | Y | Y | Y | R | R | R | R | R | R | R |
| | 12 | R | R | R | R | R | R | R | R | R | B | B | B | B | B | B | B |
| | 13 | B | B | B | B | B | B | B | B | B | G | G | G | G | G | G | G |
| | 18 | G | G | G | Y | Y | Y | R | R | R | Y | Y | Y | R | R | R | B |
| | 24 | B | Y | R | B | G | R | B | G | Y | B | G | R | B | G | Y | G |
| | | SAMPLE ID | | | | | | | | | | | | | | | |
| | | | | | | | | | | | | A | B | C | D | E | F |
| | | Q | R | S | T | U | V | W | X | Y | Z | A | B | C | D | E | F |
| SPOT | 1 | G | G | Y | Y | Y | Y | Y | Y | Y | Y | Y | R | R | R | R | R |
| ID | 2 | Y | Y | R | R | R | R | R | R | R | R | R | B | B | B | B | B |
| | 3 | R | R | B | B | B | B | B | B | B | B | B | G | G | G | G | G |
| | 4 | B | B | G | G | G | G | G | G | G | G | G | Y | Y | Y | Y | Y |
| | 5 | G | G | Y | Y | Y | Y | Y | Y | Y | Y | Y | R | R | R | R | R |
| | 6 | Y | Y | R | R | R | R | R | R | R | R | R | B | B | B | B | B |
| | 7 | R | R | B | B | B | B | B | B | B | B | B | G | G | G | G | G |
| | 8 | B | B | G | G | G | G | G | G | G | G | G | Y | Y | Y | Y | Y |
| | 10 | Y | Y | R | R | R | R | R | R | R | R | R | B | B | B | B | B |
| | 11 | R | R | B | B | B | B | B | B | B | B | B | G | G | G | G | G |
| | 12 | B | B | G | G | G | G | G | G | G | G | G | Y | Y | Y | Y | Y |
| | 13 | G | G | Y | Y | Y | Y | Y | Y | Y | Y | Y | R | R | R | R | R |
| | 18 | B | B | R | R | R | B | B | B | G | G | G | B | B | B | G | G |
| | 24 | Y | R | B | G | Y | G | Y | R | B | Y | R | G | Y | R | B | Y |

Dye colors coupled to oligos for each sample for 32 sample-plex assay (B: “Blue” = Alexa 488, G: “Green” = Alexa 546, Y: “Yellow” = Texas Red-X, and R: “Red” = Alexa 647). Spot ID numbers 1 to 8 and 10 to 12 were used to identify the target nucleic acid. Spot ID numbers 13, 18, and 24 were used to identify the sample.

The contents of Tables 4 and 5 are similar to the contents of Tables 2 and 3, respectively. Thus, DV2 tag-418, as an example, which has an underlying spot sequence of 3-5-10-13-18-24, would comprise (in order) a first position hybridized to a plurality of yellow fluorophore labeled oligonucleotides, a second position hybridized to a plurality of blue fluorophore labeled oligonucleotides, and a third position hybridized to a plurality of green fluorophore labeled oligonucleotides; the first through third positions are for identifying a target nucleic acid. The DV2 tag-418 would identify the sample as Sample A if it further comprises (in order) a fourth position hybridized to a plurality of a blue fluorophore labeled oligonucleotides, a fifth position hybridized to a plurality of a green fluorophore labeled oligonucleotides, and a sixth position hybridized to a plurality of a blue fluorophore labeled oligonucleotides. However, the DV2 tag-418 would identify the sample as Sample B if it instead further comprises fourth, fifth and six positions that are hybridized to a plurality of blue fluorophore labeled oligonucleotides, green fluorophore labeled oligonucleotides, and yellow fluorophore labeled oligonucleotides.

### OTHER EMBODIMENTS

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

What is claimed is:

1. A single-stranded nucleic acid probe comprising at least three regions:
at least a first region capable of binding to a target nucleic acid in a sample;
at least a second region capable of binding to at least a first plurality of labeled single-stranded oligonucleotides, wherein the first plurality of labeled single-stranded oligonucleotides identifies the target nucleic acid in the sample; and
at least a third region capable of binding to at least a second plurality of labeled single-stranded oligonucleotides, wherein the second plurality of labeled single-stranded oligonucleotides identifies the sample.

2. The single-stranded nucleic acid probe of claim 1, wherein the target nucleic acid is a synthetic oligonucleotide.

3. The single-stranded nucleic acid probe of claim 1 or claim 2, wherein the target nucleic acid is obtained from a biological sample.

4. The single-stranded nucleic acid probe of any one of claims 1 to 3, wherein the second region comprises at least two positions for binding to at least two first pluralities of labeled single-stranded oligonucleotides.

5. The single-stranded nucleic acid probe of claim 4, wherein the second region comprises at least three positions for binding to at least three first pluralities of labeled single-stranded oligonucleotides.

6. The single-stranded nucleic acid probe of claim 5, wherein the second region comprises at least four positions for binding to at least four first pluralities of labeled single-stranded oligonucleotides.

7. The single-stranded nucleic acid probe of claim 6, wherein the second region comprises at least five positions for binding to at least five first pluralities of labeled single-stranded oligonucleotides.

8. The single-stranded nucleic acid probe of claim 7, wherein the second region comprises at least six positions for binding to at least six first pluralities of labeled single-stranded oligonucleotides.

9. The single-stranded nucleic acid probe of claim 8, wherein the second region comprises at least ten positions for binding to at least ten first pluralities of labeled single-stranded oligonucleotides.

10. The single-stranded nucleic acid probe of any one of claims 1 to 9, wherein the first plurality of labeled single-stranded oligonucleotides comprises or is complementary to one or more of SEQ ID NO: 1 to SEQ ID NO: 808.

11. The single-stranded nucleic acid probe of any one of claims 1 to 10, wherein the third region comprises at least two positions for binding to at least two second pluralities of labeled single-stranded oligonucleotides.

12. The single-stranded nucleic acid probe of claim 4, wherein the third region comprises at least two positions for binding to at least two second pluralities of labeled single-stranded oligonucleotides.

13. The single-stranded nucleic acid probe of claim 5, wherein the third region comprises at least two positions for binding to at least two second pluralities of labeled single-stranded oligonucleotides.

14. The single-stranded nucleic acid probe of claim 6, wherein the third region comprises at least two positions for binding to at least two second pluralities of labeled single-stranded oligonucleotides.

15. The single-stranded nucleic acid probe of claim 7, wherein the third region comprises at least two positions for binding to at least two second pluralities of labeled single-stranded oligonucleotides.

16. The single-stranded nucleic acid probe of claim 8, wherein the third region comprises at least two positions for binding to at least two second pluralities of labeled single-stranded oligonucleotides.

17. The single-stranded nucleic acid probe of claim 9, wherein the third region comprises at least two positions for binding to at least two second pluralities of labeled single-stranded oligonucleotides.

18. The single-stranded nucleic acid probe of any one of claims 1 to 17, wherein the third region comprises at least three positions for binding to at least three second pluralities of labeled single-stranded oligonucleotides.

19. The single-stranded nucleic acid probe of claim 4, wherein the third region comprises at least three positions for binding to at least three second pluralities of labeled single-stranded oligonucleotides.

20. The single-stranded nucleic acid probe of claim 5, wherein the third region comprises at least three positions for binding to at least three second pluralities of labeled single-stranded oligonucleotides.

21. The single-stranded nucleic acid probe of claim 6, wherein the third region comprises at least three positions for binding to at least three second pluralities of labeled single-stranded oligonucleotides.

22. The single-stranded nucleic acid probe of claim 7, wherein the third region comprises at least three positions for binding to at least three second pluralities of labeled single-stranded oligonucleotides.

23. The single-stranded nucleic acid probe of claim 8, wherein the third region comprises at least three positions for binding to at least three second pluralities of labeled single-stranded oligonucleotides.

24. The single-stranded nucleic acid probe of claim 9, wherein the third region comprises at least three positions for binding to at least three second pluralities of labeled single-stranded oligonucleotides.

25. The single-stranded nucleic acid probe of any one of claims 1 to 24, wherein the third region comprises at least four positions for binding to at least four second pluralities of labeled single-stranded oligonucleotides.

26. The single-stranded nucleic acid probe of claim 4, wherein the third region comprises at least four positions for binding to at least four second pluralities of labeled single-stranded oligonucleotides.

27. The single-stranded nucleic acid probe of claim 5, wherein the third region comprises at least four positions for binding to at least four second pluralities of labeled single-stranded oligonucleotides.

28. The single-stranded nucleic acid probe of claim 6, wherein the third region comprises at least four positions for binding to at least four second pluralities of labeled single-stranded oligonucleotides.

29. The single-stranded nucleic acid probe of claim 7, wherein the third region comprises at least four positions for binding to at least four second pluralities of labeled single-stranded oligonucleotides.

30. The single-stranded nucleic acid probe of claim 8, wherein the third region comprises at least four positions for binding to at least four second pluralities of labeled single-stranded oligonucleotides.

31. The single-stranded nucleic acid probe of claim 9, wherein the third region comprises at least four positions for binding to at least four second pluralities of labeled single-stranded oligonucleotides.

32. The single-stranded nucleic acid probe of any one of claims 1 to 31, wherein the third region comprises at least five positions for binding to at least five second pluralities of labeled single-stranded oligonucleotides.

33. The single-stranded nucleic acid probe of any one of claims 1 to 32, wherein the third region comprises at least six positions for binding to at least six second pluralities of labeled single-stranded oligonucleotides.

34. The single-stranded nucleic acid probe of any one of claims 1 to 33, wherein the third region comprises at least ten positions for binding to at least ten second pluralities of labeled single-stranded oligonucleotides.

35. The single-stranded nucleic acid probe of any one of claims 1 to 34, wherein the second plurality of labeled single-stranded oligonucleotides comprises or is complementary to one or more of SEQ ID NO: 1 to SEQ ID NO: 808.

36. The single-stranded nucleic acid probe of any one of claims 1 to 35, wherein the labeled single-stranded oligonucleotide comprises deoxyribonucleotides.

37. The single-stranded nucleic acid probe of any one of claims 1 to 36, wherein the labeled single-stranded oligonucleotide comprises a label monomer selected from the group consisting of a fluorochrome, quantum dot, dye, enzyme, nanoparticle, chemiluminescent marker, biotin, and another monomer that can be detected directly or indirectly.

38. The single-stranded nucleic acid probe of claim 4, wherein a label monomer at a first position of the second region is spectrally or spatially distinguishable from a label monomer at a second position of the second region.

39. The single-stranded nucleic acid probe of claim 11, wherein a label monomer at a first position of the third region is spectrally or spatially distinguishable from a label monomer at a second position of the third region.

40. The single-stranded nucleic acid probe of any one of claims 1 to 39, wherein a label monomer at a position of the second region that is adjacent to a position of the third region differs from a label monomer at the position of the third region that is adjacent to the position of the second region and wherein the label monomers are spectrally or spatially distinguishable.

41. The single-stranded nucleic acid probe of any one of claims 1 to 40, wherein at least one labeled single-stranded oligonucleotide has a melting/hybridization temperatures of between about 65° C. and about 85° C.

42. The single-stranded nucleic acid probe of any one of claims 1 to 41, wherein at least one labeled single-stranded oligonucleotide has a melting/hybridization temperatures of about 80° C.

43. The single-stranded nucleic acid probe of any one of claims 1 to 41 further comprising a single-stranded or double-stranded RNA, DNA, PNA, or other polynucleotide analogue spacer between the first region and the second region and/or between the first region and the third region.

44. A composition comprising at least two single-stranded nucleic acid probes, comprising
(a) at least a first single-stranded nucleic acid probe comprising at least three regions:
at least a first region capable of binding to a first sequence of a target nucleic acid in a sample;
at least a second region capable of binding to at least a first plurality of labeled single-stranded oligonucleotides, wherein the first plurality of labeled single-stranded oligonucleotides identifies the target nucleic acid in the sample; and
at least a third region capable of binding to at least a second plurality of labeled single-stranded oligonucleotides, wherein the second plurality of labeled single-stranded oligonucleotides identifies the sample; and
(b) at least a second single-stranded nucleic acid probe comprising at least two regions:
at least a first region capable of binding to a second sequence of the target nucleic acid in a sample, wherein the first and the second sequences of the target nucleic acid are different or capable of binding to a second target nucleic acid; and
at least a second region comprising at least one affinity moiety.

45. The composition of claim 44, wherein the target nucleic acid is a synthetic oligonucleotide.

46. The composition of claim 44 or claim 45, wherein the target nucleic acid is obtained from a biological sample.

47. The composition of any one of claims 44 to 47, wherein the at least one affinity moiety is biotin, avidin, or streptavidin.

48. The composition of any one of claims 44 to 47, wherein the target nucleic acid in a sample is a portion of a first protein probe that is released from or present in the first protein probe which includes a first region capable of binding to first target protein in a sample and a second region including a partially double-stranded nucleic acid or including a single-stranded nucleic acid.

49. A composition comprising a plurality of single-stranded nucleic acid probes, each single-stranded nucleic acid probe comprising at least three regions:
at least a first region capable of binding to a target nucleic acid in a sample;
at least a second region capable of binding to a first plurality of labeled single-stranded oligonucleotides, wherein the first plurality of labeled single-stranded oligonucleotides identifies the target nucleic acid in the sample; and
at least a third region capable of binding to a second plurality of labeled single-stranded oligonucleotides, wherein the second plurality of labeled single-stranded oligonucleotides identifies the sample;
wherein the plurality of single-stranded nucleic acid probes are capable of binding to different target nucleic acids obtained from the same sample or the plurality of single-stranded nucleic acid probes are capable of binding to the same target nucleic acid obtained from different samples.

50. The composition of claim 49, wherein each target nucleic acid in a sample is a portion of a first protein probe that is released from or present in the first protein probe which includes a first region capable of binding to first target protein in a sample and a second region including a partially double-stranded nucleic acid or including a single-stranded nucleic acid.

51. A method for simultaneously detecting a target nucleic acid in at least two samples comprising:
(1) contacting a first sample with one or more first single-stranded nucleic acid probes capable of identifying a first target nucleic acid and the first sample;
(2) contacting the first sample with a first plurality of labeled single-stranded oligonucleotides capable of binding to the first single-stranded nucleic acid probes that can identify the first target nucleic acid,
(3) contacting the first sample with a second plurality of labeled single-stranded oligonucleotides capable of binding to the first single-stranded nucleic acid probes that can identify the first sample;
(4) contacting at least a second sample with one or more at least second single-stranded nucleic acid probes capable of identifying the first target nucleic acid and the at least second sample
(5) contacting the at least second sample with the first plurality of labeled single-stranded oligonucleotides capable of binding to the at least second single-stranded nucleic acid probes that can identify the first target nucleic acid,
(6) contacting the at least second sample with at least a third plurality of labeled single-stranded oligonucleotides capable of binding to the at least second single-stranded nucleic acid probes that can identify the at least second sample;
wherein the first sample and the at least second sample are different;
(7) pooling the sample of step (3) and the sample of step (6) to form a combined sample; and
(8) detecting the first and the at least second single-stranded nucleic acid probes in the combined sample, thereby simultaneously detecting the target nucleic acid in at least two samples.

52. The method of claim 51, wherein the first target nucleic acid is a synthetic oligonucleotide.

53. The method of claim 51 or claim 52 wherein the first target nucleic acid is obtained from a biological sample.

54. The method of any one of claims 51 to 53, wherein the first target nucleic acid is a portion of a first protein probe that is released from or present in the first protein probe which includes a first region capable of binding to first target protein in a sample and a second region including a partially double-stranded nucleic acid or including a single-stranded nucleic acid.

55. The method of claim any one of claims 49 to 51, further comprising contacting the first and at least second sample with at least a third single-stranded nucleic acid probe comprising at least two regions:
at least a first region capable of binding to a second sequence of the first target nucleic acid in a sample, wherein the first and the second sequences of the first target nucleic acid are different or capable of binding to a second target nucleic acid; and
at least a second region comprising at least one affinity moiety.

56. A method for simultaneously detecting a target nucleic acid in at least two samples comprising:
(1) contacting a first sample with one or more first single-stranded nucleic acid probes capable of identifying a first target nucleic acid and the first sample to form one or more first complexes, wherein the first single-stranded nucleic acid probes comprise a first plurality of labeled single-stranded oligonucleotides that can identify the first target nucleic acid;
(2) contacting the one or more first complexes with a second plurality of labeled single-stranded oligonucleotides capable of binding to the first single-stranded nucleic acid probes that can identify the first sample;
(3) contacting at least a second sample with one or more at least second single-stranded nucleic acid probes capable of identifying the first target nucleic acid and the at least second sample to form one or more second complexes, wherein the at least second single-stranded nucleic acid probes comprise the first plurality of labeled single-stranded oligonucleotides that can identify the first target nucleic acid;
(4) contacting the one or more second complexes with at least a third plurality of labeled single-stranded oligonucleotides capable of binding to the at least second single-stranded nucleic acid probes that can identify the at least second sample;
wherein the first sample and the at least second sample are different;
(5) pooling the sample of step (2) and the sample of step (4) to form a combined sample; and
(6) detecting the first and the at least second single-stranded nucleic acid probes in the combined sample, thereby simultaneously detecting the target nucleic acid in at least two samples.

57. The method of claim 56, wherein the first target nucleic acid is a portion of a first protein probe that is released from or present in the first protein probe which includes a first region capable of binding to first target protein in a sample and a second region including a partially double-stranded nucleic acid or including a single-stranded nucleic acid.

58. A method for simultaneously detecting a target nucleic acid in at least two samples comprising:
(1) contacting a first sample with one or more first single-stranded nucleic acid probes capable of identifying a first target nucleic acid and the first sample, wherein the first single-stranded nucleic acid probes comprise a first plurality of labeled single-stranded oligonucleotides that can identify the first target nucleic acid and a second plurality of labeled single-stranded oligonucleotides that can identify the first sample;
(2) contacting at least a second sample with one or more at least second single-stranded nucleic acid probes capable of identifying the first target nucleic acid and the at least second sample, wherein the at least second single-stranded nucleic acid probes comprise the first plurality of labeled single-stranded oligonucleotides that can identify the first target nucleic acid and at least a third plurality of labeled single-stranded oligonucleotides that can identify the at least second sample;
wherein the first sample and the at least second sample are different;
(3) pooling the sample of step (1) and the sample of step (2) to form a combined sample; and
(4) detecting the first and the at least second single-stranded nucleic acid probes in the combined sample, thereby simultaneously detecting the target nucleic acid in at least two samples.

59. The method of claim 58, wherein the first target nucleic acid is a portion of a first protein probe that is released from or present in the first protein probe which includes a first region capable of binding to first target protein in a sample and a second region including a partially double-stranded nucleic acid or including a single-stranded nucleic acid.

60. A method for simultaneously detecting a target nucleic acid in at least two samples comprising:
(1) contacting one or more first single-stranded nucleic acid probes capable of identifying a first target nucleic acid and the first sample with a second plurality of labeled single-stranded oligonucleotides capable of binding to the first single-stranded nucleic acid probes that can identify the first sample to form one or more first complexes, wherein the first single-stranded nucleic acid probes comprise a first plurality of labeled single-stranded oligonucleotides that can identify the first target nucleic acid;
(2) contacting the one or more first complexes with the first sample;
(3) contacting one or more at least second single-stranded nucleic acid probes capable of identifying the first target nucleic acid and the at least second sample with at least a third plurality of labeled single-stranded oligonucleotides capable of binding to the at least second single-stranded nucleic acid probes that can identify the at least second sample to form one or more second complexes, wherein the second single-stranded nucleic acid probes comprise the first plurality of labeled single-stranded oligonucleotides that can identify the first target nucleic acid;
(4) contacting the one or more second complexes with at least a second sample;
wherein the first sample and the at least second sample are different;
(5) pooling the sample of step (2) and the sample of step (4) to form a combined sample; and
(6) detecting the first and the at least second single-stranded nucleic acid probes in the combined sample, thereby simultaneously detecting the target nucleic acid in at least two samples.

61. The method of claim 60, wherein the first target nucleic acid is a portion of a first protein probe that is released from or present in the first protein probe which includes a first region capable of binding to first target protein in a sample and a second region including a partially double-stranded nucleic acid or including a single-stranded nucleic acid.

62. A kit comprising
a first container comprising
a plurality of single-stranded nucleic acid probes, each single-stranded nucleic acid probe comprising at least three regions:
at least a first region capable of binding to a first target nucleic acid;
at least a second region capable of binding to a first plurality of labeled single-stranded oligonucleotides, wherein the first plurality of labeled single-stranded oligonucleotides identifies the first target nucleic acid; and
at least a third region capable of binding to at least a second or third plurality of labeled single-stranded oligonucleotides; and
the first plurality of labeled single-stranded oligonucleotides;
a second container comprising the second plurality of labeled single-stranded oligonucleotides that can identify the first sample; and
at least a third container comprising at least the third plurality of labeled single-stranded oligonucleotides that can identify the at least second sample.

63. The kit of claim 62, wherein the target nucleic acid is a synthetic oligonucleotide.

64. The kit of claim 62 or claim 63, wherein the target nucleic acid is obtained from a biological sample.

65. The kit of any one of claims 62 to 64, further comprising a second single-stranded nucleic acid probe or a plurality of second single-stranded probes each probe comprising at least two regions:
at least a first region capable of binding to a second sequence of the first target nucleic acid in a sample, wherein the first and the second sequences of the first target nucleic acid are different or capable of binding to a second target nucleic acid; and
at least a second region comprising at least one affinity moiety.

66. The kit of claim any one of claims 62 to 65 further comprising at least a fourth container comprising a plurality of first protein probes each protein probe comprising a first region capable of binding to a target protein in a sample and a second region comprising a partially double-stranded nucleic acid or including a single-stranded nucleic acid.

67. A kit comprising
a first container comprising a plurality of single-stranded nucleic acid probes, each single-stranded nucleic acid probe comprising at least three regions:
at least a first region capable of binding to a target nucleic acid;
at least a second region capable of binding to a first plurality of labeled single-stranded oligonucleotides, wherein the first plurality of labeled single-stranded oligonucleotides identifies the target nucleic acid; and
at least a third region capable of binding to at least a second or third plurality of labeled single-stranded oligonucleotides;
a second container comprising the first plurality of labeled single-stranded oligonucleotides;
a third container comprising the second plurality of labeled single-stranded oligonucleotides that can identify the first sample; and
at least a fourth container comprising at least the third plurality of labeled single-stranded oligonucleotides that can identify the at least second sample.

68. The kit of claim 67 further comprising at least a fifth container comprising a plurality of first protein probes each protein probe comprising a first region capable of binding to a target protein in a sample and a second region comprising a partially double-stranded nucleic acid or including a single-stranded nucleic acid.

69. A kit comprising
a first container comprising
a plurality of single-stranded nucleic acid probes, each single-stranded nucleic acid probe comprising at least three regions:
at least a first region capable of binding to a target nucleic acid;
at least a second region capable of binding to a first plurality of labeled single-stranded oligonucleotides, wherein the first plurality of labeled single-stranded oligonucleotides identifies the target nucleic acid; and
at least a third region capable of binding to at least a second or third plurality of labeled single-stranded oligonucleotides;
the first plurality of labeled single-stranded oligonucleotides; and
the second plurality of labeled single-stranded oligonucleotides that can identify a first sample
at least a second container comprising
the plurality of single-stranded nucleic acid probes;
the first plurality of labeled single-stranded oligonucleotides; and
the at least third plurality of labeled single-stranded oligonucleotides that can identify at least a second sample.

70. The kit of claim 69 further comprising at least a third container comprising a plurality of first protein probes each protein probe comprising a first region capable of binding to a target protein in a sample and a second region comprising a partially double-stranded nucleic acid or including a single-stranded nucleic acid.

71. A single-stranded nucleic acid probe comprising at least two regions:
at least a first region capable of binding to a target nucleic acid in a sample; and
at least a second region capable of binding to at least a plurality of labeled single-stranded oligonucleotides, wherein the plurality of labeled single-stranded oligonucleotides identifies the sample.

72. The single-stranded nucleic acid probe of claim 71, wherein the target nucleic acid is a synthetic oligonucleotide.

73. The single-stranded nucleic acid probe of claim 71 or claim 72, wherein the target nucleic acid is obtained from a biological sample.

74. The single-stranded nucleic acid probe of any one of claims 71 to 73, wherein the second region comprises at least two positions for binding to at least two pluralities of labeled single-stranded oligonucleotides.

75. The single-stranded nucleic acid probe of claim 74, wherein the second region comprises at least three positions for binding to at least three pluralities of labeled single-stranded oligonucleotides.

76. The single-stranded nucleic acid probe of claim 75, wherein the second region comprises at least four positions for binding to at least four pluralities of labeled single-stranded oligonucleotides.

77. The single-stranded nucleic acid probe of claim 76, wherein the second region comprises at least five positions for binding to at least five pluralities of labeled single-stranded oligonucleotides.

78. The single-stranded nucleic acid probe of claim 77, wherein the second region comprises at least six positions for binding to at least six pluralities of labeled single-stranded oligonucleotides.

79. The single-stranded nucleic acid probe of claim 78, wherein the second region comprises at least ten positions for binding to at least ten pluralities of labeled single-stranded oligonucleotides.

80. The single-stranded nucleic acid probe of any one of claims 71 to 79, wherein the plurality of labeled single-stranded oligonucleotides comprises or is complementary to one or more of SEQ ID NO: 1 to SEQ ID NO: 808.

81. The single-stranded nucleic acid probe of any one of claims 71 to 80, wherein the labeled single-stranded oligonucleotide comprises deoxyribonucleotides.

82. The single-stranded nucleic acid probe of any one of claims 71 to 81, wherein the labeled single-stranded oligonucleotide comprises a label monomer selected from the group consisting of a fluorochrome, quantum dot, dye, enzyme, nanoparticle, chemiluminescent marker, biotin, and another monomer that can be detected directly or indirectly.

83. The single-stranded nucleic acid probe of any one of claims 71 to 82, wherein a label monomer at a first position of the second region is spectrally or spatially distinguishable from a label monomer at a second position of the second region.

84. The single-stranded nucleic acid probe of any one of claims 71 to 83, wherein at least one labeled single-stranded oligonucleotide has a melting/hybridization temperatures of between about 65° C. and about 85° C.

85. The single-stranded nucleic acid probe of any one of claims 71 to 84, wherein at least one labeled single-stranded oligonucleotide has a melting/hybridization temperatures of about 80° C.

86. The single-stranded nucleic acid probe of any one of claims 71 to 85 further comprising a single-stranded or double-stranded RNA, DNA, PNA, or other polynucleotide analogue spacer between the first region and the second region and/or between the first region and the third region.


SEQUENCE LISTING (813 sequences)

SEQ ID NO: 1 - DNA, 35 bp, Artificial Sequence
  Synthetic polynucleotide 1 (B1DV1)
aggtagacaaaagtaagccagtggcacagtgagga

SEQ ID NO: 2 - DNA, 27 bp, Artificial Sequence
  Synthetic polynucleotide 1 (B1DV1)
agatgagcgagctgaggacaatgacgg

SEQ ID NO: 3 - DNA, 27 bp, Artificial Sequence
  Synthetic polynucleotide 1 (B1DV1)
agtcggaggaatcagagcggtgagaca

SEQ ID NO: 4 - DNA, 38 bp, Artificial Sequence
  Synthetic polynucleotide 1 (B1DV1)
agtggaggatatcaaagataagagcatagggaaatgca

SEQ ID NO: 5 - DNA, 28 bp, Artificial Sequence
  Synthetic polynucleotide 1 (B1DV1)
acaatggaaacgtcccaaggtggaagcg

SEQ ID NO: 6 - DNA, 39 bp, Artificial Sequence
  Synthetic polynucleotide 1 (B1DV1)
tgggagaatgaagaggtaagcaaatagaagacgtaggga

SEQ ID NO: 7 - DNA, 36 bp, Artificial Sequence
  Synthetic polynucleotide 1 (B1DV1)
acatgaaaccatgcagaagataagaaaatgccagaa

SEQ ID NO: 8 - DNA, 37 bp, Artificial Sequence
  Synthetic polynucleotide 1 (B1DV1)
tacgacggtgagagaaatcaaccagtacaagcgctga

SEQ ID NO: 9 - DNA, 30 bp, Artificial Sequence
  Synthetic polynucleotide 1 (B1DV1)
acagctaccgaggtagcgagatgaacaaga

SEQ ID NO: 10 - DNA, 31 bp, Artificial Sequence
  Synthetic polynucleotide 1 (B1DV1)
tgcgaacctcaggaactcaagaagtagcgaa

SEQ ID NO: 11 - DNA, 26 bp, Artificial Sequence
  Synthetic polynucleotide 1 (B1DV1)
atcgaccgggtcgggaaagtcgagaa

SEQ ID NO: 12 - DNA, 35 bp, Artificial Sequence
  Synthetic polynucleotide 1 (B1DV1)
ataagaacgtaccagggatacagaactagggacgt

SEQ ID NO: 13 - DNA, 27 bp, Artificial Sequence
  Synthetic polynucleotide 1 (B1DV1)
aggagggtgggacgatacggcgctgaa

SEQ ID NO: 14 - DNA, 26 bp, Artificial Sequence
  Synthetic polynucleotide 1 (B1DV1)
acgggtgggagggtaacagggtggaa

SEQ ID NO: 15 - DNA, 37 bp, Artificial Sequence
  Synthetic polynucleotide 1 (B1DV1)
aagtaagagactaaggaactgaaacagctaacaggct

SEQ ID NO: 16 - DNA, 35 bp, Artificial Sequence
  Synthetic polynucleotide 1 (B1DV1)
aagggaacatggagaaataaagacactggagcgca

SEQ ID NO: 17 - DNA, 34 bp, Artificial Sequence
  Synthetic polynucleotide 1 (B1DV1)
tccggaagatagagaaaatgagagcgtgaaacca

SEQ ID NO: 18 - DNA, 31 bp, Artificial Sequence
  Synthetic polynucleotide 1 (B1DV1)
tgaaagggatcaagaggtgacggagcataga

SEQ ID NO: 19 - DNA, 31 bp, Artificial Sequence
  Synthetic polynucleotide 1 (B1DV1)
aagctgaaacaaatagggaagctgaagacca

SEQ ID NO: 20 - DNA, 31 bp, Artificial Sequence
  Synthetic polynucleotide 1 (B1DV1)
taagcgggctgccaaagataagagagtgaca

SEQ ID NO: 21 - DNA, 29 bp, Artificial Sequence
  Synthetic polynucleotide 1 (B1DV1)
agatacgcgccgtggagaagtgcaggaca

SEQ ID NO: 22 - DNA, 26 bp, Artificial Sequence
  Synthetic polynucleotide 1 (B1DV1)
taaaacaatggccgcatcaggccggg

SEQ ID NO: 23 - DNA, 34 bp, Artificial Sequence
  Synthetic polynucleotide 1 (B1DV1)
tgagggcaatacaagagctagaagagtaccgcga

SEQ ID NO: 24 - DNA, 33 bp, Artificial Sequence
  Synthetic polynucleotide 1 (B1DV1)
taggaaggtggcaccagtaaggaaataagccca

SEQ ID NO: 25 - DNA, 35 bp, Artificial Sequence
  Synthetic polynucleotide 1 (B1DV1)
tgaggacatacacgagtcgaaaaataagcgagtca

SEQ ID NO: 26 - DNA, 26 bp, Artificial Sequence
  Synthetic polynucleotide 1 (B1DV1)
aacgctaggccaactggcggcatggg

SEQ ID NO: 27 - DNA, 25 bp, Artificial Sequence
  Synthetic polynucleotide 1 (B1DV1)
acggtgcgcgggtcgacagaggtgt

SEQ ID NO: 28 - DNA, 36 bp, Artificial Sequence
  Synthetic polynucleotide 1 (B1DV1)
acaagtgacaggatgaaagcataagaaggtgacgca

SEQ ID NO: 29 - DNA, 31 bp, Artificial Sequence
  Synthetic polynucleotide 1 (B1DV1)
actagggccatacaaagagtggaccaatcca

SEQ ID NO: 30 - DNA, 30 bp, Artificial Sequence
  Synthetic polynucleotide 1 (B1DV1)
aacctgcgaagataggaggataacaccggt

SEQ ID NO: 31 - DNA, 36 bp, Artificial Sequence
  Synthetic polynucleotide 1 (B1DV1)
agggcaactacaaggatcaaaggatgaaagaataaa

SEQ ID NO: 32 - DNA, 30 bp, Artificial Sequence
  Synthetic polynucleotide 1 (B1DV1)
acactaagggcgtccaacagtaccgaagtc

SEQ ID NO: 33 - DNA, 40 bp, Artificial Sequence
  Synthetic polynucleotide 1 (B1DV1)
agggcgtcacaggctgaacagaactcaaccgaagtctaga

SEQ ID NO: 34 - DNA, 39 bp, Artificial Sequence
  Synthetic polynucleotide 2 (G1DV1)
agaaataacaggaatagcacaagtaggaacataaacaga

SEQ ID NO: 35 - DNA, 42 bp, Artificial Sequence
  Synthetic polynucleotide 2 (G1DV1)
tgcacaccaataaagagatacggaaataaagacatagagaca

SEQ ID NO: 36 - DNA, 38 bp, Artificial Sequence
  Synthetic polynucleotide 2 (G1DV1)
tccgcaaataaagggatgaaacaataaccgggtcgcga

SEQ ID NO: 37 - DNA, 27 bp, Artificial Sequence
  Synthetic polynucleotide 2 (G1DV1)
agctaacacccatcgccagctcgggca

SEQ ID NO: 38 - DNA, 36 bp, Artificial Sequence
  Synthetic polynucleotide 2 (G1DV1)
aactgatatcctcgaaggactagcaagatggaaaca

SEQ ID NO: 39 - DNA, 37 bp, Artificial Sequence
  Synthetic polynucleotide 2 (G1DV1)
tggacaagtaaaagggtgaagaagtaccacaaactca

SEQ ID NO: 40 - DNA, 34 bp, Artificial Sequence
  Synthetic polynucleotide 2 (G1DV1)
acagctaagacaagtcgggaagtaaaaaaatcca

SEQ ID NO: 41 - DNA, 28 bp, Artificial Sequence
  Synthetic polynucleotide 2 (G1DV1)
agggtaacagaatggcgaagtgagcgaa

SEQ ID NO: 42 - DNA, 32 bp, Artificial Sequence
  Synthetic polynucleotide 2 (G1DV1)
agtagagcaatgacggcatggagaaatgggaa

SEQ ID NO: 43 - DNA, 29 bp, Artificial Sequence
  Synthetic polynucleotide 2 (G1DV1)
aaaatcacgagatagacgagtgggcaggt

SEQ ID NO: 44 - DNA, 24 bp, Artificial Sequence
  Synthetic polynucleotide 2 (G1DV1)
aggcgggtgaccggaatacgacaa

SEQ ID NO: 45 - DNA, 27 bp, Artificial Sequence
  Synthetic polynucleotide 2 (G1DV1)
tgagagactgccgcagtgcgaaagtgg

SEQ ID NO: 46 - DNA, 31 bp, Artificial Sequence
  Synthetic polynucleotide 2 (G1DV1)
cgggataaaaaagctgaagggagtcaaacca

SEQ ID NO: 47 - DNA, 33 bp, Artificial Sequence
  Synthetic polynucleotide 2 (G1DV1)
tcagcgggatacagaagtagaacaatgcacaga

SEQ ID NO: 48 - DNA, 25 bp, Artificial Sequence
  Synthetic polynucleotide 2 (G1DV1)
tgcgcaggtaagcgggtgcaagact

SEQ ID NO: 49 - DNA, 32 bp, Artificial Sequence
  Synthetic polynucleotide 2 (G1DV1)
agaaaagctcggcaaatcgccgaatagacaaa

SEQ ID NO: 50 - DNA, 25 bp, Artificial Sequence
  Synthetic polynucleotide 2 (G1DV1)
tcggaaggtgagcgggtacgaagac

SEQ ID NO: 51 - DNA, 26 bp, Artificial Sequence
  Synthetic polynucleotide 2 (G1DV1)
tggcggccataacgccatgagggcat

SEQ ID NO: 52 - DNA, 34 bp, Artificial Sequence
  Synthetic polynucleotide 2 (G1DV1)
agcaaaagtacccaaataaggcagtcagagagtg

SEQ ID NO: 53 - DNA, 25 bp, Artificial Sequence
  Synthetic polynucleotide 2 (G1DV1)
acgggctacagcggctaggccgact

SEQ ID NO: 54 - DNA, 30 bp, Artificial Sequence
  Synthetic polynucleotide 2 (G1DV1)
acagcagtgcgggaagtgaaaaaagctcga

SEQ ID NO: 55 - DNA, 31 bp, Artificial Sequence
  Synthetic polynucleotide 2 (G1DV1)
aggatgcaaccatgagccaataaaggcgtcg

SEQ ID NO: 56 - DNA, 30 bp, Artificial Sequence
  Synthetic polynucleotide 2 (G1DV1)
agagctgccaagatgcgaaagtgaggacat

SEQ ID NO: 57 - DNA, 39 bp, Artificial Sequence
  Synthetic polynucleotide 2 (G1DV1)
agacgaatagaaaaatgagacgatagcgaggtgacacca

SEQ ID NO: 58 - DNA, 25 bp, Artificial Sequence
  Synthetic polynucleotide 2 (G1DV1)
taccagggtccggcaggtagccaga

SEQ ID NO: 59 - DNA, 39 bp, Artificial Sequence
  Synthetic polynucleotide 2 (G1DV1)
tagggaaatagaacgatcaaaggatagcgaaacgtcaga

SEQ ID NO: 60 - DNA, 35 bp, Artificial Sequence
  Synthetic polynucleotide 2 (G1DV1)
aactcgggagctaaaaggataacggaatcagatgt

SEQ ID NO: 61 - DNA, 26 bp, Artificial Sequence
  Synthetic polynucleotide 2 (G1DV1)
acaacgcagatcagaacctgggagca

SEQ ID NO: 62 - DNA, 37 bp, Artificial Sequence
  Synthetic polynucleotide 2 (G1DV1)
agtggagccaatcaacaagataagaaaaatgaaggca

SEQ ID NO: 63 - DNA, 22 bp, Artificial Sequence
  Synthetic polynucleotide 2 (G1DV1)
tggaaacggtccgggcagtgga

SEQ ID NO: 64 - DNA, 34 bp, Artificial Sequence
  Synthetic polynucleotide 2 (G1DV1)
acgatccaagagtaggaaaatcagaagcgtacga

SEQ ID NO: 65 - DNA, 41 bp, Artificial Sequence
  Synthetic polynucleotide 2 (G1DV1)
aactacaagcgtcagggagtagaaaagtaacacaatgaaga

SEQ ID NO: 66 - DNA, 43 bp, Artificial Sequence
  Synthetic polynucleotide 2 (G1DV1)
aactaagcacgtgaaagagtagaggaactaggaaacatctaga

SEQ ID NO: 67 - DNA, 48 bp, Artificial Sequence
  Synthetic polynucleotide 3 (Y1DV1)
agagggcgtaacaaaggtcgaaaactcaaacactacaaaaatgggaca

SEQ ID NO: 68 - DNA, 28 bp, Artificial Sequence
  Synthetic polynucleotide 3 (Y1DV1)
aatagcgcccatcggaagcctgagcgga

SEQ ID NO: 69 - DNA, 33 bp, Artificial Sequence
  Synthetic polynucleotide 3 (Y1DV1)
tagcggactacggcagtaaagagatagcggagc

SEQ ID NO: 70 - DNA, 29 bp, Artificial Sequence
  Synthetic polynucleotide 3 (Y1DV1)
tgaaagcctcaggacgtgaacaaatggga

SEQ ID NO: 71 - DNA, 29 bp, Artificial Sequence
  Synthetic polynucleotide 3 (Y1DV1)
aggataaccgggtaggagggtgaaacaga

SEQ ID NO: 72 - DNA, 33 bp, Artificial Sequence
  Synthetic polynucleotide 3 (Y1DV1)
tgagacggtaagaaaagtaagagaaggtgcgat

SEQ ID NO: 73 - DNA, 25 bp, Artificial Sequence
  Synthetic polynucleotide 3 (Y1DV1)
atcgcgggcgtcacgcaacgtgcaa

SEQ ID NO: 74 - DNA, 37 bp, Artificial Sequence
  Synthetic polynucleotide 3 (Y1DV1)
aaatgacggaataaaagaatcgaggaggtcaaggcga

SEQ ID NO: 75 - DNA, 32 bp, Artificial Sequence
  Synthetic polynucleotide 3 (Y1DV1)
taagcgcgtgagaggatagaaagatcgagcca

SEQ ID NO: 76 - DNA, 23 bp, Artificial Sequence
  Synthetic polynucleotide 3 (Y1DV1)
tagcgggccatcagcggcgtgcg

SEQ ID NO: 77 - DNA, 30 bp, Artificial Sequence
  Synthetic polynucleotide 3 (Y1DV1)
aggagtcgcaccactcaagagctaacccga

SEQ ID NO: 78 - DNA, 26 bp, Artificial Sequence
  Synthetic polynucleotide 3 (Y1DV1)
tcagcgagtacagcgggtagaagcgt

SEQ ID NO: 79 - DNA, 30 bp, Artificial Sequence
  Synthetic polynucleotide 3 (Y1DV1)
cgcgggatagaggaagtccaaagatcccga

SEQ ID NO: 80 - DNA, 28 bp, Artificial Sequence
  Synthetic polynucleotide 3 (Y1DV1)
actgccagcgtaggaacactgaccacat

SEQ ID NO: 81 - DNA, 33 bp, Artificial Sequence
  Synthetic polynucleotide 3 (Y1DV1)
agcaccatcaaaagctgaacgagatgagacact

SEQ ID NO: 82 - DNA, 28 bp, Artificial Sequence
  Synthetic polynucleotide 3 (Y1DV1)
acgcaggatgagaacgtcgcaagcatga

SEQ ID NO: 83 - DNA, 31 bp, Artificial Sequence
  Synthetic polynucleotide 3 (Y1DV1)
accgggtgcagagctaggagaataccgccga

SEQ ID NO: 84 - DNA, 26 bp, Artificial Sequence
  Synthetic polynucleotide 3 (Y1DV1)
tcaggccacgtaggaagatccaagcc

SEQ ID NO: 85 - DNA, 33 bp, Artificial Sequence
  Synthetic polynucleotide 3 (Y1DV1)
tggcacagagtcaagacgctagaaaaatgaaga

SEQ ID NO: 86 - DNA, 28 bp, Artificial Sequence
  Synthetic polynucleotide 3 (Y1DV1)
agtcagcaaagtagggagggtgggagca

SEQ ID NO: 87 - DNA, 29 bp, Artificial Sequence
  Synthetic polynucleotide 3 (Y1DV1)
tgaacgagtgaggacataggacgatccca

SEQ ID NO: 88 - DNA, 34 bp, Artificial Sequence
  Synthetic polynucleotide 3 (Y1DV1)
aagtgacggaatgaccaggtgagaaagtagggca

SEQ ID NO: 89 - DNA, 38 bp, Artificial Sequence
  Synthetic polynucleotide 3 (Y1DV1)
aatcaagcagtcaaagcgtgaagaaaactagaaggcgt

SEQ ID NO: 90 - DNA, 34 bp, Artificial Sequence
  Synthetic polynucleotide 3 (Y1DV1)
aaaagagtggagaagctccgacagatacaaaagt

SEQ ID NO: 91 - DNA, 24 bp, Artificial Sequence
  Synthetic polynucleotide 3 (Y1DV1)
agaggcctgagagggtcgggcaaa

SEQ ID NO: 92 - DNA, 30 bp, Artificial Sequence
  Synthetic polynucleotide 3 (Y1DV1)
atcccagactcggagaatcgcgacaatgca

SEQ ID NO: 93 - DNA, 27 bp, Artificial Sequence
  Synthetic polynucleotide 3 (Y1DV1)
aacgtggcgcggtgggcgaggtgccga

SEQ ID NO: 94 - DNA, 33 bp, Artificial Sequence
  Synthetic polynucleotide 3 (Y1DV1)
aatcacgcgaatggacggatatgtacactgaaa

SEQ ID NO: 95 - DNA, 28 bp, Artificial Sequence
  Synthetic polynucleotide 3 (Y1DV1)
aagctcacaaaataagcggatcgggaca

SEQ ID NO: 96 - DNA, 35 bp, Artificial Sequence
  Synthetic polynucleotide 3 (Y1DV1)
tcggaacctgagacgaatacagacgataaagcaat

SEQ ID NO: 97 - DNA, 24 bp, Artificial Sequence
  Synthetic polynucleotide 3 (Y1DV1)
aaccgactagacgagtgccagggt

SEQ ID NO: 98 - DNA, 37 bp, Artificial Sequence
  Synthetic polynucleotide 3 (Y1DV1)
aggaacgtaccaaaagtccagaagtccacgggtggac

SEQ ID NO: 99 - DNA, 34 bp, Artificial Sequence
  Synthetic polynucleotide 3 (Y1DV1)
agactagagaacagtacagaaaatgccaccctaa

SEQ ID NO: 100 - DNA, 36 bp, Artificial Sequence
  Synthetic polynucleotide 3 (Y1DV1)
acgggtaagggaatgcgggagtggacaaactctaga

SEQ ID NO: 101 - DNA, 25 bp, Artificial Sequence
  Synthetic
aggacaaatgacgggatgccaggga

SEQ ID NO: 102 - DNA, 25 bp, Artificial Sequence
  Synthetic polynucleotide 4 (R1DV1)
tgcacgactggcaggatggcaacgt

SEQ ID NO: 103 - DNA, 25 bp, Artificial Sequence
  Synthetic polynucleotide 4 (R1DV1)
aagaggctaccacgctccgggaggt

SEQ ID NO: 104 - DNA, 34 bp, Artificial Sequence
  Synthetic polynucleotide 4 (R1DV1)
aaggcgatcgaaagagtagagaagcatacccgca

SEQ ID NO: 105 - DNA, 33 bp, Artificial Sequence
  Synthetic polynucleotide 4 (R1DV1)
taggacaataaaagggtaagggaagtgggagca

SEQ ID NO: 106 - DNA, 35 bp, Artificial Sequence
  Synthetic polynucleotide 4 (R1DV1)
tccgaaaatagaaccctacaggcaagtagacaggt

SEQ ID NO: 107 - DNA, 27 bp, Artificial Sequence
  Synthetic polynucleotide 4 (R1DV1)
aaacgcatgagaagacctgggcgcggg

SEQ ID NO: 108 - DNA, 26 bp, Artificial Sequence
  Synthetic polynucleotide 4 (R1DV1)
tcggaacactgcgccagtaaggccca

SEQ ID NO: 109 - DNA, 35 bp, Artificial Sequence
  Synthetic polynucleotide 4 (R1DV1)
tgaaaaacggtgagagatatcacagaactggaaca

SEQ ID NO: 110 - DNA, 34 bp, Artificial Sequence
  Synthetic polynucleotide 4 (R1DV1)
actccggacatacagaaatacacgaatagggcaa

SEQ ID NO: 111 - DNA, 33 bp, Artificial Sequence
  Synthetic polynucleotide 4 (R1DV1)
tagacgagtaacaacgataacgcgagtagcgcg

SEQ ID NO: 112 - DNA, 34 bp, Artificial Sequence
  Synthetic polynucleotide 4 (R1DV1)
agatacgaagagtagcaagactcggaggagtcca

SEQ ID NO: 113 - DNA, 41 bp, Artificial Sequence
  Synthetic polynucleotide 4 (R1DV1)
agaaaataagcagatggcaaagatagggaagaataaacgac

SEQ ID NO: 114 - DNA, 30 bp, Artificial Sequence
  Synthetic polynucleotide 4 (R1DV1)
tggagccagtgaggagctaaaagggtcagc

SEQ ID NO: 115 - DNA, 35 bp, Artificial Sequence
  Synthetic polynucleotide 4 (R1DV1)
agactggacgggtaaggagatacaaaactcagcga

SEQ ID NO: 116 - DNA, 37 bp, Artificial Sequence
  Synthetic polynucleotide 4 (R1DV1)
atcaaaaaatacaggcaagtaccagccatccacgggt

SEQ ID NO: 117 - DNA, 25 bp, Artificial Sequence
  Synthetic polynucleotide 4 (R1DV1)
aacgggatgccaccgtccggcaaat

SEQ ID NO: 118 - DNA, 32 bp, Artificial Sequence
  Synthetic polynucleotide 4 (R1DV1)
aaaagaatccagcagctagagccggtaaccca

SEQ ID NO: 119 - DNA, 32 bp, Artificial Sequence
  Synthetic polynucleotide 4 (R1DV1)
atcccgagagtcgaagagatggcagaatgaga

SEQ ID NO: 120 - DNA, 33 bp, Artificial Sequence
  Synthetic polynucleotide 4 (R1DV1)
acaatacaagggataagcagggtacgggaatga

SEQ ID NO: 121 - DNA, 40 bp, Artificial Sequence
  Synthetic polynucleotide 4 (R1DV1)
aacggtagaggacatacgaggataacaacactaaaggact

SEQ ID NO: 122 - DNA, 37 bp, Artificial Sequence
  Synthetic polynucleotide 4 (R1DV1)
agagagaataaacagcgtgacacgactaaaaaaaggt

SEQ ID NO: 123 - DNA, 30 bp, Artificial Sequence
  Synthetic polynucleotide 4 (R1DV1)
acgggcgatcaaacaagtggaagactccaa

SEQ ID NO: 124 - DNA, 32 bp, Artificial Sequence
  Synthetic polynucleotide 4 (R1DV1)
aagataacgcggtgagcgaatacggaggtcga

SEQ ID NO: 125 - DNA, 29 bp, Artificial Sequence
  Synthetic polynucleotide 4 (R1DV1)
aaaaaatagacgggatggcgggatgccag

SEQ ID NO: 126 - DNA, 42 bp, Artificial Sequence
  Synthetic polynucleotide 4 (R1DV1)
agtagaagccctaagagaatagaaagcataaaacactaggca

SEQ ID NO: 127 - DNA, 36 bp, Artificial Sequence
  Synthetic polynucleotide 4 (R1DV1)
aacttgtacaatagacgggtgaaaagaatgaggcga

SEQ ID NO: 128 - DNA, 37 bp, Artificial Sequence
  Synthetic polynucleotide 4 (R1DV1)
tcaaagcatagcaaaatcaggaactaagcagggtcgc

SEQ ID NO: 129 - DNA, 32 bp, Artificial Sequence
  Synthetic polynucleotide 4 (R1DV1)
cggcatggaaacatagccaaatagggaggact

SEQ ID NO: 130 - DNA, 32 bp, Artificial Sequence
  Synthetic polynucleotide 4 (R1DV1)
agcagggtggaaggatgacgaactagaagcga

SEQ ID NO: 131 - DNA, 34 bp, Artificial Sequence
  Synthetic polynucleotide 4 (R1DV1)
tcaacggataaggaggtgcacagatagcacgaat

SEQ ID NO: 132 - DNA, 39 bp, Artificial Sequence
  Synthetic polynucleotide 4 (R1DV1)
aacgcgggtaagaaagtacaagaatgagcaacgtctaga

SEQ ID NO: 133 - DNA, 35 bp, Artificial Sequence
  Synthetic polynucleotide 5 (B2DV1)
agaaggatacaggaatcaagacatgcagggaacta

SEQ ID NO: 134 - DNA, 32 bp, Artificial Sequence
  Synthetic polynucleotide 5 (B2DV1)
aagacataccggaataagagaggctaaaggga

SEQ ID NO: 135 - DNA, 37 bp, Artificial Sequence
  Synthetic polynucleotide 5 (B2DV1)
taaggagagtacgaggagtaaagacagatcgacggga

SEQ ID NO: 136 - DNA, 35 bp, Artificial Sequence
  Synthetic polynucleotide 5 (B2DV1)
ataacaagagtcaagacgtgaccgacctgagcgca

SEQ ID NO: 137 - DNA, 28 bp, Artificial Sequence
  Synthetic polynucleotide 5 (B2DV1)
tcgagaccgcgggagactcggcgggtga

SEQ ID NO: 138 - DNA, 39 bp, Artificial Sequence
  Synthetic polynucleotide 5 (B2DV1)
acaaatggaaggataagaacgggtagaagaactacgcga

SEQ ID NO: 139 - DNA, 35 bp, Artificial Sequence
  Synthetic polynucleotide 5 (B2DV1)
atgaagggatgtacaaggtagggcagataagaggg

SEQ ID NO: 140 - DNA, 29 bp, Artificial Sequence
  Synthetic polynucleotide 5 (B2DV1)
tggcgagatgaaaaggctcggaaaactca

SEQ ID NO: 141 - DNA, 26 bp, Artificial Sequence
  Synthetic polynucleotide 5 (B2DV1)
accaactggagaactaggccggtcga

SEQ ID NO: 142 - DNA, 36 bp, Artificial Sequence
  Synthetic polynucleotide 5 (B2DV1)
aggaatagaaacgtaagcgagataagaggatggcgc

SEQ ID NO: 143 - DNA, 28 bp, Artificial Sequence
  Synthetic polynucleotide 5 (B2DV1)
agactgggacaatcgggaggtcgagaga

SEQ ID NO: 144 - DNA, 37 bp, Artificial Sequence
  Synthetic polynucleotide 5 (B2DV1)
tcacgggaatagacacgtccaagaaatgaacaaggta

SEQ ID NO: 145 - DNA, 31 bp, Artificial Sequence
  Synthetic polynucleotide 5 (B2DV1)
aggacatgcgggaataagaaactcaaaccct

SEQ ID NO: 146 - DNA, 30 bp, Artificial Sequence
  Synthetic polynucleotide 5 (B2DV1)
aggccagtcacagaagtaacccagtacgca

SEQ ID NO: 147 - DNA, 32 bp, Artificial Sequence
  Synthetic polynucleotide 5 (B2DV1)
agtcaccgagtcggaacgatgacaaagtaaaa

SEQ ID NO: 148 - DNA, 40 bp, Artificial Sequence
  Synthetic polynucleotide 5 (B2DV1)
aaggtgaaagaatgagagcactaaaaaagtaaaagagatc

SEQ ID NO: 149 - DNA, 26 bp, Artificial Sequence
  Synthetic polynucleotide 5 (B2DV1)
aggcgcgtgggcgagatgcagaggta

SEQ ID NO: 150 - DNA, 40 bp, Artificial Sequence
  Synthetic polynucleotide 5 (B2DV1)
acgaaataaaaagatcgcagaatccaaagactacggagga

SEQ ID NO: 151 - DNA, 43 bp, Artificial Sequence
  Synthetic polynucleotide 5 (B2DV1)
tcaggcaaataagaagataaaaaagatccgaagataacgaggt

SEQ ID NO: 152 - DNA, 32 bp, Artificial Sequence
  Synthetic polynucleotide 5 (B2DV1)
aggccaatgagagaataccgagcgtagagcca

SEQ ID NO: 153 - DNA, 42 bp, Artificial Sequence
  Synthetic polynucleotide 5 (B2DV1)
tacgagagtaaagagagtagggcagtaaaaagatccagcggt

SEQ ID NO: 154 - DNA, 24 bp, Artificial Sequence
  Synthetic polynucleotide 5 (B2DV1)
aggagcatcggcgcctcaagaaga

SEQ ID NO: 155 - DNA, 35 bp, Artificial Sequence
  Synthetic polynucleotide 5 (B2DV1)
tccagagatggaacgctaggagaataaagcggtga

SEQ ID NO: 156 - DNA, 31 bp, Artificial Sequence
  Synthetic polynucleotide 5 (B2DV1)
aagcgtacaaacgtagggaagtcgggagagt

SEQ ID NO: 157 - DNA, 41 bp, Artificial Sequence
  Synthetic polynucleotide 5 (B2DV1)
agagagatacagagagtaagagccatagacacctgagacga

SEQ ID NO: 158 - DNA, 25 bp, Artificial Sequence
  Synthetic polynucleotide 5 (B2DV1)
tcggcaactgggcaacatccagaga

SEQ ID NO: 159 - DNA, 28 bp, Artificial Sequence
  Synthetic polynucleotide 5 (B2DV1)
tgggcacctaggcacatcaccgggtgca

SEQ ID NO: 160 - DNA, 36 bp, Artificial Sequence
  Synthetic polynucleotide 5 (B2DV1)
aaggatcacagagtagaacgctcaaagaagtcacca

SEQ ID NO: 161 - DNA, 24 bp, Artificial Sequence
  Synthetic polynucleotide 5 (B2DV1)
agtgcacgggtaagggacatgcga

SEQ ID NO: 162 - DNA, 26 bp, Artificial Sequence
  Synthetic polynucleotide 5 (B2DV1)
aggtgagagcgggtggaaaactcgac

SEQ ID NO: 163 - DNA, 28 bp, Artificial Sequence
  Synthetic polynucleotide 5 (B2DV1)
agctcaacacatgagggaatgccagaga

SEQ ID NO: 164 - DNA, 28 bp, Artificial Sequence
  Synthetic polynucleotide 5 (B2DV1)
tggagcaaataagagacatgccgggcga

SEQ ID NO: 165 - DNA, 28 bp, Artificial Sequence
  Synthetic polynucleotide 5 (B2DV1)
tgaggaactacaacagtagcccaatgca

SEQ ID NO: 166 - DNA, 34 bp, Artificial Sequence
  Synthetic polynucleotide 5 (B2DV1)
aggctaaggaagtacaaaagtcgggaaaggatcc

SEQ ID NO: 167 - DNA, 36 bp, Artificial Sequence
  Synthetic polynucleotide 6 (G2DV1)
agaatgaaacagtggaaaggtcggaaactgaggcgg

SEQ ID NO: 168 - DNA, 33 bp, Artificial Sequence
  Synthetic polynucleotide 6 (G2DV1)
agtagaaacaagtcgcgagatacgagagatgaa

SEQ ID NO: 169 - DNA, 31 bp, Artificial Sequence
  Synthetic polynucleotide 6 (G2DV1)
agagcgtcaaagaatggaggactcagaagat

SEQ ID NO: 170 - DNA, 31 bp, Artificial Sequence
  Synthetic polynucleotide 6 (G2DV1)
agaaacatcgggacaggtgagaacactggga

SEQ ID NO: 171 - DNA, 40 bp, Artificial Sequence
  Synthetic polynucleotide 6 (G2DV1)
aagaatcgaaaaaactaccaaaactacaaaaactgcaaaa

SEQ ID NO: 172 - DNA, 32 bp, Artificial Sequence
  Synthetic polynucleotide 6 (G2DV1)
atgaaaggagtggaccactggacagccgcgga

SEQ ID NO: 173 - DNA, 29 bp, Artificial Sequence
  Synthetic polynucleotide 6 (G2DV1)
agctcaagaactgtgtacaggtgcagcgg

SEQ ID NO: 174 - DNA, 33 bp, Artificial Sequence
  Synthetic polynucleotide 6 (G2DV1)
tcagaaacatcaaaaggtaacagcatcgcggga

SEQ ID NO: 175 - DNA, 31 bp, Artificial Sequence
  Synthetic polynucleotide 6 (G2DV1)
tcggaaaatccgggagtcaaaaagtgcagaa

SEQ ID NO: 176 - DNA, 37 bp, Artificial Sequence
  Synthetic polynucleotide 6 (G2DV1)
atgacacgggtacgaaacctcagaaacataaacagct

SEQ ID NO: 177 - DNA, 34 bp, Artificial Sequence
  Synthetic polynucleotide 6 (G2DV1)
acgggcatgagagcataaagagataaaaaaatgc

SEQ ID NO: 178 - DNA, 29 bp, Artificial Sequence
  Synthetic polynucleotide 6 (G2DV1)
agagcgataccacacgtaaggagatcgca

SEQ ID NO: 179 - DNA, 35 bp, Artificial Sequence
  Synthetic polynucleotide 6 (G2DV1)
aggtgccacgatcagaaaatagagacatcgacggg

SEQ ID NO: 180 - DNA, 26 bp, Artificial Sequence
  Synthetic polynucleotide 6 (G2DV1)
tggcgaactgagcaacatccgggaat

SEQ ID NO: 181 - DNA, 26 bp, Artificial Sequence
  Synthetic polynucleotide 6 (G2DV1)
aagaacctcaagggcgtgcaaccggt

SEQ ID NO: 182 - DNA, 30 bp, Artificial Sequence
  Synthetic polynucleotide 6 (G2DV1)
acacgaatgagaggatgcgggagtcagcgg

SEQ ID NO: 183 - DNA, 35 bp, Artificial Sequence
  Synthetic polynucleotide 6 (G2DV1)
agtgggaaggtgaggaaagtagaaaaaatacagcc

SEQ ID NO: 184 - DNA, 29 bp, Artificial Sequence
  Synthetic polynucleotide 6 (G2DV1)
aggtacaaaagtaaagcgggtcaaccggt

SEQ ID NO: 185 - DNA, 26 bp, Artificial Sequence
  Synthetic polynucleotide 6 (G2DV1)
agccaaggtgcgaaagtaggcccgtg

SEQ ID NO: 186 - DNA, 39 bp, Artificial Sequence
  Synthetic polynucleotide 6 (G2DV1)
agaaaatccggcaatagaacagtaaaaaggtgagacgca

SEQ ID NO: 187 - DNA, 32 bp, Artificial Sequence
  Synthetic polynucleotide 6 (G2DV1)
taacacgggtgcaaagctaagagagatccacc

SEQ ID NO: 188 - DNA, 43 bp, Artificial Sequence
  Synthetic polynucleotide 6 (G2DV1)
agtaggaagatgggaaagcataacaaagatagaagaataaggc

SEQ ID NO: 189 - DNA, 38 bp, Artificial Sequence
  Synthetic polynucleotide 6 (G2DV1)
agtaggagaatgaaaacaatacagaaaagtagggcaga

SEQ ID NO: 190 - DNA, 32 bp, Artificial Sequence
  Synthetic polynucleotide 6 (G2DV1)
taaagcactaacgagctgacaggctggcagga

SEQ ID NO: 191 - DNA, 31 bp, Artificial Sequence
  Synthetic polynucleotide 6 (G2DV1)
tgaagacatgacacggatcaaggcatggacg

SEQ ID NO: 192 - DNA, 26 bp, Artificial Sequence
  Synthetic polynucleotide 6 (G2DV1)
agtgcacaggtcaagcgctccggcga

SEQ ID NO: 193 - DNA, 29 bp, Artificial Sequence
  Synthetic polynucleotide 6 (G2DV1)
tgagcgaatagcccgcgtaacacagtccg

SEQ ID NO: 194 - DNA, 27 bp, Artificial Sequence
  Synthetic polynucleotide 6 (G2DV1)
acaaggtgggagcatagcccgatcacg

SEQ ID NO: 195 - DNA, 28 bp, Artificial Sequence
  Synthetic polynucleotide 6 (G2DV1)
agggtgcaggcgtgagaaaagatccagg

SEQ ID NO: 196 - DNA, 37 bp, Artificial Sequence
  Synthetic polynucleotide 6 (G2DV1)
gctaaaacactagaaacatggcacagtacagggactg

SEQ ID NO: 197 - DNA, 27 bp, Artificial Sequence
  Synthetic polynucleotide 6 (G2DV1)
aggagctgacaagatgcacacggtcaa

SEQ ID NO: 198 - DNA, 38 bp, Artificial Sequence
  Synthetic polynucleotide 6 (G2DV1)
aggataagaaacgtcacgaggataccaccatcacgaaa

SEQ ID NO: 199 - DNA, 33 bp, Artificial Sequence
  Synthetic polynucleotide 6 (G2DV1)
taagcgggtcggaaacatgccagactgggcaag

SEQ ID NO: 200 - DNA, 39 bp, Artificial Sequence
  Synthetic polynucleotide 6 (G2DV1)
tcaccaggatgaagaaatgaagaagtagcaagaggatcc

SEQ ID NO: 201 - DNA, 32 bp, Artificial Sequence
  Synthetic polynucleotide 7 (Y2DV1)
agaaaaacataagcagcatggagcgctacggg

SEQ ID NO: 202 - DNA, 25 bp, Artificial Sequence
  Synthetic polynucleotide 7 (Y2DV1)
aggatggcacggtcgcgggaatcga

SEQ ID NO: 203 - DNA, 28 bp, Artificial Sequence
  Synthetic polynucleotide 7 (Y2DV1)
acgctgaagcaatcaaagggtagggagg

SEQ ID NO: 204 - DNA, 28 bp, Artificial Sequence
  Synthetic polynucleotide 7 (Y2DV1)
tcgggcaatacagcgatcggaggatgag

SEQ ID NO: 205 - DNA, 29 bp, Artificial Sequence
  Synthetic polynucleotide 7 (Y2DV1)
acgggtcacaagaaatgggaccatccgca

SEQ ID NO: 206 - DNA, 44 bp, Artificial Sequence
  Synthetic polynucleotide 7 (Y2DV1)
aatcaagagataaggaaatacaaggaataagaaggatgaaaacg

SEQ ID NO: 207 - DNA, 35 bp, Artificial Sequence
  Synthetic polynucleotide 7 (Y2DV1)
tgcggagatgaagacgatgacgacaataatgtaca

SEQ ID NO: 208 - DNA, 31 bp, Artificial Sequence
  Synthetic polynucleotide 7 (Y2DV1)
atgagcagatacacacggtgaaccccgcgga

SEQ ID NO: 209 - DNA, 45 bp, Artificial Sequence
  Synthetic polynucleotide 7 (Y2DV1)
acgctaacaaaataccgaaataaaaaaatagcgagatacagggct

SEQ ID NO: 210 - DNA, 29 bp, Artificial Sequence
  Synthetic polynucleotide 7 (Y2DV1)
aaagaagtgcaagggagtaaggccctggc

SEQ ID NO: 211 - DNA, 28 bp, Artificial Sequence
  Synthetic polynucleotide 7 (Y2DV1)
agggtaacaagctcgcggacgtcaccgc

SEQ ID NO: 212 - DNA, 30 bp, Artificial Sequence
  Synthetic polynucleotide 7 (Y2DV1)
cgtgcgggcaatacgaccgctaaagaagct

SEQ ID NO: 213 - DNA, 33 bp, Artificial Sequence
  Synthetic polynucleotide 7 (Y2DV1)
agagagaatcagcagggtaaagaagtaggaccg

SEQ ID NO: 214 - DNA, 35 bp, Artificial Sequence
  Synthetic polynucleotide 7 (Y2DV1)
actcgcaagatcgagaaataaaaaagtggaccggg

SEQ ID NO: 215 - DNA, 35 bp, Artificial Sequence
  Synthetic polynucleotide 7 (Y2DV1)
tgagcacggatgaacaagtgaagaaatacagaggt

SEQ ID NO: 216 - DNA, 36 bp, Artificial Sequence
  Synthetic polynucleotide 7 (Y2DV1)
acaagaatcagaaacctcgggacatagaaacatcca

SEQ ID NO: 217 - DNA, 34 bp, Artificial Sequence
  Synthetic polynucleotide 7 (Y2DV1)
agaagatgggcagaatcgaacaggtagacgagtg

SEQ ID NO: 218 - DNA, 34 bp, Artificial Sequence
  Synthetic polynucleotide 7 (Y2DV1)
agaagactaaaacaatgaagaactaggcccgcat

SEQ ID NO: 219 - DNA, 37 bp, Artificial Sequence
  Synthetic polynucleotide 7 (Y2DV1)
acgagactgaaagcatcaagaggataaggaactcaga

SEQ ID NO: 220 - DNA, 36 bp, Artificial Sequence
  Synthetic polynucleotide 7 (Y2DV1)
aaactagagaaactaaggcgatcaggaggatgagaa

SEQ ID NO: 221 - DNA, 28 bp, Artificial Sequence
  Synthetic polynucleotide 7 (Y2DV1)
aatcgacgagtaagggaagtccgggagt

SEQ ID NO: 222 - DNA, 31 bp, Artificial Sequence
  Synthetic polynucleotide 7 (Y2DV1)
acacggatgaaccgatacaaggatggcgacg

SEQ ID NO: 223 - DNA, 26 bp, Artificial Sequence
  Synthetic polynucleotide 7 (Y2DV1)
tccagcaatggaaaggctcagccgat

SEQ ID NO: 224 - DNA, 37 bp, Artificial Sequence
  Synthetic polynucleotide 7 (Y2DV1)
aacacggtaggagcaatagaagaaataaagacgtgcg

SEQ ID NO: 225 - DNA, 42 bp, Artificial Sequence
  Synthetic polynucleotide 7 (Y2DV1)
cggaataagaaaggataacgagatcaagacaatggaacgagt

SEQ ID NO: 226 - DNA, 38 bp, Artificial Sequence
  Synthetic polynucleotide 7 (Y2DV1)
acaaaagtcaaaacgtcgaaagggtaaagcactgacga

SEQ ID NO: 227 - DNA, 33 bp, Artificial Sequence
  Synthetic polynucleotide 7 (Y2DV1)
acatagagcgataaacacctgggaagatccgga

SEQ ID NO: 228 - DNA, 36 bp, Artificial Sequence
  Synthetic polynucleotide 7 (Y2DV1)
actagcaaaatcacgacaaatgcaaagatcagccga

SEQ ID NO: 229 - DNA, 35 bp, Artificial Sequence
  Synthetic polynucleotide 7 (Y2DV1)
tcgaggcataccagggtgaaaaaatcaaaaaagta

SEQ ID NO: 230 - DNA, 30 bp, Artificial Sequence
  Synthetic polynucleotide 7 (Y2DV1)
accggacctaaaccaacgtgagaaaatgcg

SEQ ID NO: 231 - DNA, 31 bp, Artificial Sequence
  Synthetic polynucleotide 7 (Y2DV1)
agcgtacagacctgacgaaatcaacaaatga

SEQ ID NO: 232 - DNA, 38 bp, Artificial Sequence
  Synthetic polynucleotide 7 (Y2DV1)
agaagtaacagagatccaaaactgaaaaggtaaaagca

SEQ ID NO: 233 - DNA, 32 bp, Artificial Sequence
  Synthetic polynucleotide 7 (Y2DV1)
tggacacgtaagcaagatagacaaggggatcc

SEQ ID NO: 234 - DNA, 34 bp, Artificial Sequence
  Synthetic polynucleotide 8 (R2DV1)
agaggtagcacacggtgaaaagctaagaacctca

SEQ ID NO: 235 - DNA, 30 bp, Artificial Sequence
  Synthetic polynucleotide 8 (R2DV1)
aacgatcgcaccatgacgcgaatgagacaa

SEQ ID NO: 236 - DNA, 37 bp, Artificial Sequence
  Synthetic polynucleotide 8 (R2DV1)
atgccgaaatacacacatacgaaacctcaagacccta

SEQ ID NO: 237 - DNA, 32 bp, Artificial Sequence
  Synthetic polynucleotide 8 (R2DV1)
acgggctaaaagagtcggagccatagaaaggt

SEQ ID NO: 238 - DNA, 28 bp, Artificial Sequence
  Synthetic polynucleotide 8 (R2DV1)
aacagcgataccgaaatggaacgggtgc

SEQ ID NO: 239 - DNA, 38 bp, Artificial Sequence
  Synthetic polynucleotide 8 (R2DV1)
agcaataaagaagctaaacgaagtaggaaaatagggaa

SEQ ID NO: 240 - DNA, 29 bp, Artificial Sequence
  Synthetic polynucleotide 8 (R2DV1)
atggaaccagtgggacatgtacagacgaa

SEQ ID NO: 241 - DNA, 26 bp, Artificial Sequence
  Synthetic polynucleotide 8 (R2DV1)
tcggaaacatgacgacctggacgggt

SEQ ID NO: 242 - DNA, 41 bp, Artificial Sequence
  Synthetic polynucleotide 8 (R2DV1)
acggcaagtaaaaaaataaaagagtagagacatgaaaacat

SEQ ID NO: 243 - DNA, 26 bp, Artificial Sequence
  Synthetic polynucleotide 8 (R2DV1)
accgcggtgagagcgtagaagcgtga

SEQ ID NO: 244 - DNA, 29 bp, Artificial Sequence
  Synthetic polynucleotide 8 (R2DV1)
aacagtaagcggctaagggagtcggagga

SEQ ID NO: 245 - DNA, 33 bp, Artificial Sequence
  Synthetic polynucleotide 8 (R2DV1)
taaacggctgacggagactgacaaccataaagc

SEQ ID NO: 246 - DNA, 29 bp, Artificial Sequence
  Synthetic polynucleotide 8 (R2DV1)
aggtgcaaagggatggcaacgtcaaggcg

SEQ ID NO: 247 - DNA, 36 bp, Artificial Sequence
  Synthetic polynucleotide 8 (R2DV1)
tcgcagaagtaagggaatggcaaaactaagcaaagt

SEQ ID NO: 248 - DNA, 34 bp, Artificial Sequence
  Synthetic polynucleotide 8 (R2DV1)
agagcactaacaaaggtaggacgaagtacgaagg

SEQ ID NO: 249 - DNA, 25 bp, Artificial Sequence
  Synthetic polynucleotide 8 (R2DV1)
tgaggaggtggaaagctggcgcaca

SEQ ID NO: 250 - DNA, 37 bp, Artificial Sequence
  Synthetic polynucleotide 8 (R2DV1)
tgagagaatacaagaactagcgaaggtacagcactgg

SEQ ID NO: 251 - DNA, 31 bp, Artificial Sequence
  Synthetic polynucleotide 8 (R2DV1)
acggatcaaaacggtcgcaacgtaagacggt

SEQ ID NO: 252 - DNA, 33 bp, Artificial Sequence
  Synthetic polynucleotide 8 (R2DV1)
agggcggtagaaaagctcaacgactgagaggca

SEQ ID NO: 253 - DNA, 31 bp, Artificial Sequence
  Synthetic polynucleotide 8 (R2DV1)
tgaggacgctaaagggagtagcaagctgaga

SEQ ID NO: 254 - DNA, 36 bp, Artificial Sequence
  Synthetic polynucleotide 8 (R2DV1)
aaatgccggaagtgaaaaactggaaagataagcaag

SEQ ID NO: 255 - DNA, 32 bp, Artificial Sequence
  Synthetic polynucleotide 8 (R2DV1)
tccgccaagtcgagaacgtagaagagtacggg

SEQ ID NO: 256 - DNA, 31 bp, Artificial Sequence
  Synthetic polynucleotide 8 (R2DV1)
agtacagaacctgggcgaagatcaggcgagt

SEQ ID NO: 257 - DNA, 33 bp, Artificial Sequence
  Synthetic polynucleotide 8 (R2DV1)
agcagagtcaaaaactggcaagatcaggagcta

SEQ ID NO: 258 - DNA, 27 bp, Artificial Sequence
  Synthetic polynucleotide 8 (R2DV1)
accaaggctcaggcaatgcaagactga

SEQ ID NO: 259 - DNA, 29 bp, Artificial Sequence
  Synthetic polynucleotide 8 (R2DV1)
acgggtcacaagcgtcaggacatagaaga

SEQ ID NO: 260 - DNA, 36 bp, Artificial Sequence
  Synthetic polynucleotide 8 (R2DV1)
atgaaagagtaccagaatacacgggatagcagacgt

SEQ ID NO: 261 - DNA, 28 bp, Artificial Sequence
  Synthetic polynucleotide 8 (R2DV1)
aaggacgataggagcgtgagccaatccg

SEQ ID NO: 262 - DNA, 37 bp, Artificial Sequence
  Synthetic polynucleotide 8 (R2DV1)
aggatagaggaataaagcacataagcgggtgagacca

SEQ ID NO: 263 - DNA, 34 bp, Artificial Sequence
  Synthetic polynucleotide 8 (R2DV1)
tgagaggatcgaagaatacgggaataaggcgggt

SEQ ID NO: 264 - DNA, 33 bp, Artificial Sequence
  Synthetic polynucleotide 8 (R2DV1)
aagaacgtgacgcaagtgaggcgataagaacaa

SEQ ID NO: 265 - DNA, 33 bp, Artificial Sequence
  Synthetic polynucleotide 8 (R2DV1)
tagcgcgaatgacggagtacccaaatcaaggga

SEQ ID NO: 266 - DNA, 37 bp, Artificial Sequence
  Synthetic polynucleotide 8 (R2DV1)
tcgccgggtgaaaaagtaagagaatcgccagcgtcca

SEQ ID NO: 267 - DNA, 37 bp, Artificial Sequence
  Synthetic polynucleotide 8 (R2DV1)
aggaagtagaggaatacaacaagtcacggaaggatcc

SEQ ID NO: 268 - DNA, 36 bp, Artificial Sequence
  Synthetic polynucleotide 9 (B3DV1)
agatcgggagataaagggagtaaacgcatcaaacga

SEQ ID NO: 269 - DNA, 31 bp, Artificial Sequence
  Synthetic polynucleotide 9 (B3DV1)
tggcgagatgaacaggatgacagggtcccga

SEQ ID NO: 270 - DNA, 24 bp, Artificial Sequence
  Synthetic polynucleotide 9 (B3DV1)
acataccgggatggagcgctggga

SEQ ID NO: 271 - DNA, 31 bp, Artificial Sequence
  Synthetic polynucleotide 9 (B3DV1)
acctggaggaactagaaacctccgaagctcc

SEQ ID NO: 272 - DNA, 36 bp, Artificial Sequence
  Synthetic polynucleotide 9 (B3DV1)
acgggtgaacaggtggacaggtaaaagaatacaaca

SEQ ID NO: 273 - DNA, 29 bp, Artificial Sequence
  Synthetic polynucleotide 9 (B3DV1)
agtacaaccgtgaaaaaccctcaggcggt

SEQ ID NO: 274 - DNA, 30 bp, Artificial Sequence
  Synthetic polynucleotide 9 (B3DV1)
agggaactcgaagaaatgagcgggtaagga

SEQ ID NO: 275 - DNA, 32 bp, Artificial Sequence
  Synthetic polynucleotide 9 (B3DV1)
aggtaaagggatgaacgggtggaaagatggga

SEQ ID NO: 276 - DNA, 31 bp, Artificial Sequence
  Synthetic polynucleotide 9 (B3DV1)
aggtaaagagaatgggacaccataaccgcat

SEQ ID NO: 277 - DNA, 26 bp, Artificial Sequence
  Synthetic polynucleotide 9 (B3DV1)
acgcggaatggcaagagtgcacaact

SEQ ID NO: 278 - DNA, 33 bp, Artificial Sequence
  Synthetic polynucleotide 9 (B3DV1)
aggagaagtcacaaagtggaaaactcgcaccgt

SEQ ID NO: 279 - DNA, 26 bp, Artificial Sequence
  Synthetic polynucleotide 9 (B3DV1)
aaggacctggacgggaatagacggga

SEQ ID NO: 280 - DNA, 34 bp, Artificial Sequence
  Synthetic polynucleotide 9 (B3DV1)
tcgaacggatatcgagtacggaaagtccaagagc

SEQ ID NO: 281 - DNA, 31 bp, Artificial Sequence
  Synthetic polynucleotide 9 (B3DV1)
tgcgacactagaacacatgcacgactacgcc

SEQ ID NO: 282 - DNA, 26 bp, Artificial Sequence
  Synthetic polynucleotide 9 (B3DV1)
aggtggaaagcgtaaacgccgtgcaa

SEQ ID NO: 283 - DNA, 29 bp, Artificial Sequence
  Synthetic polynucleotide 9 (B3DV1)
agctcgcccggaatcagacagtgggcgga

SEQ ID NO: 284 - DNA, 27 bp, Artificial Sequence
  Synthetic polynucleotide 9 (B3DV1)
taacgaaagtaacccagggtgacgcgc

SEQ ID NO: 285 - DNA, 35 bp, Artificial Sequence
  Synthetic polynucleotide 9 (B3DV1)
tgccagactaaaggagtaagggagatacaggcact

SEQ ID NO: 286 - DNA, 31 bp, Artificial Sequence
  Synthetic polynucleotide 9 (B3DV1)
aaaagggataagcggatgacacggatagcaa

SEQ ID NO: 287 - DNA, 34 bp, Artificial Sequence
  Synthetic polynucleotide 9 (B3DV1)
acgtaaggagatgacaggcatagcagaactacga

SEQ ID NO: 288 - DNA, 37 bp, Artificial Sequence
  Synthetic polynucleotide 9 (B3DV1)
aaatgaacgaatagagaggctaagagggtggagacga

SEQ ID NO: 289 - DNA, 38 bp, Artificial Sequence
  Synthetic polynucleotide 9 (B3DV1)
tgcaaagaataagagaataaaagacgtgcaggcatcga

SEQ ID NO: 290 - DNA, 26 bp, Artificial Sequence
  Synthetic polynucleotide 9 (B3DV1)
aaactgcaagagtagggccatcggca

SEQ ID NO: 291 - DNA, 29 bp, Artificial Sequence
  Synthetic polynucleotide 9 (B3DV1)
actcgaccgggtacaacgctaacgaacgt

SEQ ID NO: 292 - DNA, 28 bp, Artificial Sequence
  Synthetic polynucleotide 9 (B3DV1)
acagggctcggcgaatcgacaaactcga

SEQ ID NO: 293 - DNA, 30 bp, Artificial Sequence
  Synthetic polynucleotide 9 (B3DV1)
aggatcagaccacatggaaacgtcgggaca

SEQ ID NO: 294 - DNA, 36 bp, Artificial Sequence
  Synthetic polynucleotide 9 (B3DV1)
ataacggaataagcaactacgacggtgaaggcgtca

SEQ ID NO: 295 - DNA, 29 bp, Artificial Sequence
  Synthetic polynucleotide 9 (B3DV1)
acaagtggacgagtaaaggcgtgaagacg

SEQ ID NO: 296 - DNA, 25 bp, Artificial Sequence
  Synthetic polynucleotide 9 (B3DV1)
tccagagctggaaccctgcgggcat

SEQ ID NO: 297 - DNA, 32 bp, Artificial Sequence
  Synthetic polynucleotide 9 (B3DV1)
aaagagagagctcaatgcgcagactgaaacaa

SEQ ID NO: 298 - DNA, 33 bp, Artificial Sequence
  Synthetic polynucleotide 9 (B3DV1)
taaccaggtagaggagatccaccggtcaggcga

SEQ ID NO: 299 - DNA, 34 bp, Artificial Sequence
  Synthetic polynucleotide 9 (B3DV1)
taaagaaagtcgacaggtgacgaggtgacgaggt

SEQ ID NO: 300 - DNA, 32 bp, Artificial Sequence
  Synthetic polynucleotide 9 (B3DV1)
acgcgaataaaccaataccggaatgagaaggt

SEQ ID NO: 301 - DNA, 42 bp, Artificial Sequence
  Synthetic polynucleotide 9 (B3DV1)
acaggcgtagaaaagatgaggagatcagagcgatgaagagat

SEQ ID NO: 302 - DNA, 40 bp, Artificial Sequence
  Synthetic polynucleotide 9 (B3DV1)
agcgaagtaaagaaaatacgaaagtccgggagaactcgag

SEQ ID NO: 303 - DNA, 33 bp, Artificial Sequence
  Synthetic polynucleotide 10 (G3DV1)
agagctacaagactacaagagtcgcgcgccgta

SEQ ID NO: 304 - DNA, 30 bp, Artificial Sequence
  Synthetic polynucleotide 10 (G3DV1)
aaaaaagtggcgcaggatggaaacgtgagg

SEQ ID NO: 305 - DNA, 34 bp, Artificial Sequence
  Synthetic polynucleotide 10 (G3DV1)
aggtagcgaaggtacagacctggagagatcagga

SEQ ID NO: 306 - DNA, 29 bp, Artificial Sequence
  Synthetic polynucleotide 10 (G3DV1)
actggcagaatgaggacatcagccgatga

SEQ ID NO: 307 - DNA, 30 bp, Artificial Sequence
  Synthetic polynucleotide 10 (G3DV1)
acgcctgggcaaataacgcaatagaagagt

SEQ ID NO: 308 - DNA, 29 bp, Artificial Sequence
  Synthetic polynucleotide 10 (G3DV1)
aggcagggtagagcgctaacggaatccgc

SEQ ID NO: 309 - DNA, 32 bp, Artificial Sequence
  Synthetic polynucleotide 10 (G3DV1)
acgataagagcgtgggaggctaacagaatggc

SEQ ID NO: 310 - DNA, 37 bp, Artificial Sequence
  Synthetic polynucleotide 10 (G3DV1)
ccaatgacgggacgtaagcagatgaaaacagtggcgg

SEQ ID NO: 311 - DNA, 28 bp, Artificial Sequence
  Synthetic polynucleotide 10 (G3DV1)
agctggaagaatcagagcagtccgaggg

SEQ ID NO: 312 - DNA, 33 bp, Artificial Sequence
  Synthetic polynucleotide 10 (G3DV1)
tgcagaggtaaggagatgggagaaatgcaaaga

SEQ ID NO: 313 - DNA, 37 bp, Artificial Sequence
  Synthetic polynucleotide 10 (G3DV1)
atgcggaagtaaacaaataagaggatcggagggctaa

SEQ ID NO: 314 - DNA, 28 bp, Artificial Sequence
  Synthetic polynucleotide 10 (G3DV1)
aacgggtcggcgagtgaaaaaatcggga

SEQ ID NO: 315 - DNA, 35 bp, Artificial Sequence
  Synthetic polynucleotide 10 (G3DV1)
agtcagcgcataaaggcctgaaaaagataagccga

SEQ ID NO: 316 - DNA, 29 bp, Artificial Sequence
  Synthetic polynucleotide 10 (G3DV1)
tgaagaaactgggcggatgacgcgggtga

SEQ ID NO: 317 - DNA, 30 bp, Artificial Sequence
  Synthetic polynucleotide 10 (G3DV1)
aagatatccgggaatggcacggtggcggga

SEQ ID NO: 318 - DNA, 27 bp, Artificial Sequence
  Synthetic polynucleotide 10 (G3DV1)
tggaaaagtgcgggaatcgggccgtga

SEQ ID NO: 319 - DNA, 29 bp, Artificial Sequence
  Synthetic polynucleotide 10 (G3DV1)
acgaatagaacgggtacacgcgataggca

SEQ ID NO: 320 - DNA, 30 bp, Artificial Sequence
  Synthetic polynucleotide 10 (G3DV1)
agataaccggatcgagagcactcaagcgat

SEQ ID NO: 321 - DNA, 25 bp, Artificial Sequence
  Synthetic polynucleotide 10 (G3DV1)
accaagctcgcaagagtaggagcgt

SEQ ID NO: 322 - DNA, 32 bp, Artificial Sequence
  Synthetic polynucleotide 10 (G3DV1)
accgggagtgacaagataagagagtagggaga

SEQ ID NO: 323 - DNA, 32 bp, Artificial Sequence
  Synthetic polynucleotide 10 (G3DV1)
tccagaagtcacgacatcgggagctgccggga

SEQ ID NO: 324 - DNA, 34 bp, Artificial Sequence
  Synthetic polynucleotide 10 (G3DV1)
tcagagggtcagggaatagcgaaaaatgcaagaa

SEQ ID NO: 325 - DNA, 33 bp, Artificial Sequence
  Synthetic polynucleotide 10 (G3DV1)
tagaaacatgcagacatgaaccgggtaagccaa

SEQ ID NO: 326 - DNA, 34 bp, Artificial Sequence
  Synthetic polynucleotide 10 (G3DV1)
tgaaccactgacagaggtgagagcatagcgagct

SEQ ID NO: 327 - DNA, 29 bp, Artificial Sequence
  Synthetic polynucleotide 10 (G3DV1)
aggacggtaaaagaggtccaacgcggtcc

SEQ ID NO: 328 - DNA, 40 bp, Artificial Sequence
  Synthetic polynucleotide 10 (G3DV1)
agagatccgaaaatcgaaagagtaaaaagataggaaggtg

SEQ ID NO: 329 - DNA, 30 bp, Artificial Sequence
  Synthetic polynucleotide 10 (G3DV1)
acccactgcgagggatgacaacgtgaaaaa

SEQ ID NO: 330 - DNA, 35 bp, Artificial Sequence
  Synthetic polynucleotide 10 (G3DV1)
aatggcgcgataacaacagtacgaaagctacggga

SEQ ID NO: 331 - DNA, 33 bp, Artificial Sequence
  Synthetic polynucleotide 10 (G3DV1)
actgacagggtcgagctctagcaaaactgcggg

SEQ ID NO: 332 - DNA, 34 bp, Artificial Sequence
  Synthetic polynucleotide 10 (G3DV1)
agtggaacggtagaggagtagggcaatagaaaca

SEQ ID NO: 333 - DNA, 32 bp, Artificial Sequence
  Synthetic polynucleotide 10 (G3DV1)
tgaagcaatgaaagccgtagggaaactccacc

SEQ ID NO: 334 - DNA, 39 bp, Artificial Sequence
  Synthetic polynucleotide 10 (G3DV1)
agtcggagaatacgacaggtcacgcaaataaagccgtca

SEQ ID NO: 335 - DNA, 44 bp, Artificial Sequence
  Synthetic polynucleotide 10 (G3DV1)
acgggtgaccggctacaagcgtgcaacaaaataagaacagtggg

SEQ ID NO: 336 - DNA, 36 bp, Artificial Sequence
  Synthetic polynucleotide 10 (G3DV1)
acagagtaggccactagaaccataaccaaactcgag

SEQ ID NO: 337 - DNA, 37 bp, Artificial Sequence
  Synthetic polynucleotide 11 (Y3DV1)
agactacaaaaatgcacagatggcgaggtaaaaaggt

SEQ ID NO: 338 - DNA, 24 bp, Artificial Sequence
  Synthetic polynucleotide 11 (Y3DV1)
agccaagctgcgcgggtgagcgga

SEQ ID NO: 339 - DNA, 29 bp, Artificial Sequence
  Synthetic polynucleotide 11 (Y3DV1)
tggcgaagtgcacaggtaagagagtacac

SEQ ID NO: 340 - DNA, 33 bp, Artificial Sequence
  Synthetic polynucleotide 11 (Y3DV1)
agctcaagcagtagggcaagtaaagaactcaca

SEQ ID NO: 341 - DNA, 27 bp, Artificial Sequence
  Synthetic polynucleotide 11 (Y3DV1)
agataagccgatcaaggcgtccaagcg

SEQ ID NO: 342 - DNA, 27 bp, Artificial Sequence
  Synthetic polynucleotide 11 (Y3DV1)
tggcgaggtgggagactacaacacgat

SEQ ID NO: 343 - DNA, 40 bp, Artificial Sequence
  Synthetic polynucleotide 11 (Y3DV1)
agagcaggtaaaaaagtaaaaagataacaaaaatccggga

SEQ ID NO: 344 - DNA, 35 bp, Artificial Sequence
  Synthetic polynucleotide 11 (Y3DV1)
agtgaaggaatcaaacaatggagaagtgaggcaac

SEQ ID NO: 345 - DNA, 29 bp, Artificial Sequence
  Synthetic polynucleotide 11 (Y3DV1)
tggagaactcagagcatacggcagatgga

SEQ ID NO: 346 - DNA, 31 bp, Artificial Sequence
  Synthetic polynucleotide 11 (Y3DV1)
acggtccgggaaatcaagcgagtgagaggga

SEQ ID NO: 347 - DNA, 31 bp, Artificial Sequence
  Synthetic polynucleotide 11 (Y3DV1)
tcgggaaataggcagaatcaaacaagtggga

SEQ ID NO: 348 - DNA, 29 bp, Artificial Sequence
  Synthetic polynucleotide 11 (Y3DV1)
agatcaaccggtgaggagactaaacgcat

SEQ ID NO: 349 - DNA, 26 bp, Artificial Sequence
  Synthetic polynucleotide 11 (Y3DV1)
aaccggatagagccgctacggcacta

SEQ ID NO: 350 - DNA, 32 bp, Artificial Sequence
  Synthetic polynucleotide 11 (Y3DV1)
agaccggtagggagctgcgggagtgcgagaca

SEQ ID NO: 351 - DNA, 27 bp, Artificial Sequence
  Synthetic polynucleotide 11 (Y3DV1)
tggacgagtggacacatcgagaggtca

SEQ ID NO: 352 - DNA, 33 bp, Artificial Sequence
  Synthetic polynucleotide 11 (Y3DV1)
aggagatagagaggactgagggactcccaggat

SEQ ID NO: 353 - DNA, 34 bp, Artificial Sequence
  Synthetic polynucleotide 11 (Y3DV1)
aaaaaggagtaagagagtccggcaggtcccagat

SEQ ID NO: 354 - DNA, 33 bp, Artificial Sequence
  Synthetic polynucleotide 11 (Y3DV1)
atcggacgtcaaacgataaaaaaatgggcggat

SEQ ID NO: 355 - DNA, 26 bp, Artificial Sequence
  Synthetic polynucleotide 11 (Y3DV1)
acagacgggtcaagaaatcgcacggc

SEQ ID NO: 356 - DNA, 35 bp, Artificial Sequence
  Synthetic polynucleotide 11 (Y3DV1)
tcggacgatgacggaataggaaaatgaggcgctaa

SEQ ID NO: 357 - DNA, 36 bp, Artificial Sequence
  Synthetic polynucleotide 11 (Y3DV1)
aaaactagagaaatgaggaagtacgagcgtcgcgga

SEQ ID NO: 358 - DNA, 43 bp, Artificial Sequence
  Synthetic polynucleotide 11 (Y3DV1)
atgaggaaatacaaggataggcaaatgagacgatggaggcgca

SEQ ID NO: 359 - DNA, 31 bp, Artificial Sequence
  Synthetic polynucleotide 11 (Y3DV1)
tcgacaagtcagaaaaatgccgcgatcgagg

SEQ ID NO: 360 - DNA, 29 bp, Artificial Sequence
  Synthetic polynucleotide 11 (Y3DV1)
acgtcgacagagtaggacactgcgggaaa

SEQ ID NO: 361 - DNA, 37 bp, Artificial Sequence
  Synthetic polynucleotide 11 (Y3DV1)
taaaggaatcagaaggtagagagcgtcacgcggtgga

SEQ ID NO: 362 - DNA, 29 bp, Artificial Sequence
  Synthetic polynucleotide 11 (Y3DV1)
aagctcagcgcaatcacggactagaagga

SEQ ID NO: 363 - DNA, 36 bp, Artificial Sequence
  Synthetic polynucleotide 11 (Y3DV1)
atccggacatcgagaaactacgagaagtgaaaaagt

SEQ ID NO: 364 - DNA, 28 bp, Artificial Sequence
  Synthetic polynucleotide 11 (Y3DV1)
aagccgatccagacatgcccacaatggc

SEQ ID NO: 365 - DNA, 32 bp, Artificial Sequence
  Synthetic polynucleotide 11 (Y3DV1)
accataacagagctcaaaactggcgaggagtg

SEQ ID NO: 366 - DNA, 33 bp, Artificial Sequence
  Synthetic polynucleotide 11 (Y3DV1)
agccacagtaagacagtcccgaaataaacacat

SEQ ID NO: 367 - DNA, 31 bp, Artificial Sequence
  Synthetic polynucleotide 11 (Y3DV1)
acaaggctagaaaaatgcgaagagtgcgaga

SEQ ID NO: 368 - DNA, 43 bp, Artificial Sequence
  Synthetic polynucleotide 11 (Y3DV1)
aggtcgggaaggtagagagaatacaaggctgacggagtgagga

SEQ ID NO: 369 - DNA, 38 bp, Artificial Sequence
  Synthetic polynucleotide 11 (Y3DV1)
aataagagagtggacagagtacgagagagtgcgaccaa

SEQ ID NO: 370 - DNA, 38 bp, Artificial Sequence
  Synthetic polynucleotide 11 (Y3DV1)
taaacgggtaggcgaatcaacggataggagaactcgag

SEQ ID NO: 371 - DNA, 29 bp, Artificial Sequence
  Synthetic polynucleotide 12 (R3DV1)
agagccagtcaagcaatcgggagaatggc

SEQ ID NO: 372 - DNA, 28 bp, Artificial Sequence
  Synthetic polynucleotide 12 (R3DV1)
accgtgggcggctaagcaaagtacgaaa

SEQ ID NO: 373 - DNA, 29 bp, Artificial Sequence
  Synthetic polynucleotide 12 (R3DV1)
aataacaacatgcgggaatcggagcgtcc

SEQ ID NO: 374 - DNA, 29 bp, Artificial Sequence
  Synthetic polynucleotide 12 (R3DV1)
agaagtcaaggaatggcaagggtgcgcaa

SEQ ID NO: 375 - DNA, 35 bp, Artificial Sequence
  Synthetic polynucleotide 12 (R3DV1)
atggaaaggtaagagggatgaacccatagagaagg

SEQ ID NO: 376 - DNA, 33 bp, Artificial Sequence
  Synthetic polynucleotide 12 (R3DV1)
tgacgggatgagaacgctaagaaaatcccaaaa

SEQ ID NO: 377 - DNA, 31 bp, Artificial Sequence
  Synthetic polynucleotide 12 (R3DV1)
taggagagataggagggtggacgaaatgcca

SEQ ID NO: 378 - DNA, 34 bp, Artificial Sequence
  Synthetic polynucleotide 12 (R3DV1)
agatgacgcaatgacagaagtaacgggaagtgac

SEQ ID NO: 379 - DNA, 28 bp, Artificial Sequence
  Synthetic polynucleotide 12 (R3DV1)
agactggcccacaatgcagaggtaaggc

SEQ ID NO: 380 - DNA, 30 bp, Artificial Sequence
  Synthetic polynucleotide 12 (R3DV1)
cctcaaccgaatcgggcgatgaaacactga

SEQ ID NO: 381 - DNA, 34 bp, Artificial Sequence
  Synthetic polynucleotide 12 (R3DV1)
aaacgtagaaaagtgcagcgactagcaggagtaa

SEQ ID NO: 382 - DNA, 36 bp, Artificial Sequence
  Synthetic polynucleotide 12 (R3DV1)
aacgatgcccgaataaaaacctaaaggagtgggaga

SEQ ID NO: 383 - DNA, 38 bp, Artificial Sequence
  Synthetic polynucleotide 12 (R3DV1)
atccacgagtaagaaaatcaaggagtaaccgactcaga

SEQ ID NO: 384 - DNA, 33 bp, Artificial Sequence
  Synthetic polynucleotide 12 (R3DV1)
aagatgaccaaggtcaaggactcaaaagctcgg

SEQ ID NO: 385 - DNA, 30 bp, Artificial Sequence
  Synthetic polynucleotide 12 (R3DV1)
agagtaacccagcgtcacaaaatgagagcc

SEQ ID NO: 386 - DNA, 34 bp, Artificial Sequence
  Synthetic polynucleotide 12 (R3DV1)
tgcaaaaatagaaacatgcggcaacctgaaacgc

SEQ ID NO: 387 - DNA, 26 bp, Artificial Sequence
  Synthetic polynucleotide 12 (R3DV1)
tgccagacagtgggaacgtccacagg

SEQ ID NO: 388 - DNA, 32 bp, Artificial Sequence
  Synthetic polynucleotide 12 (R3DV1)
tggagggatgggaaagtacaacactgaccaga

SEQ ID NO: 389 - DNA, 34 bp, Artificial Sequence
  Synthetic polynucleotide 12 (R3DV1)
tcggaaaatggcaacactacgaaggtggatatct

SEQ ID NO: 390 - DNA, 39 bp, Artificial Sequence
  Synthetic polynucleotide 12 (R3DV1)
agcagaatgaaaagggtaaagagactaaagcaatccaga

SEQ ID NO: 391 - DNA, 34 bp, Artificial Sequence
  Synthetic polynucleotide 12 (R3DV1)
agtacaacgataggagcgtacgagaatgcaaaga

SEQ ID NO: 392 - DNA, 37 bp, Artificial Sequence
  Synthetic polynucleotide 12 (R3DV1)
tgaacgggtacgacaagtgaaaaactcaagagataca

SEQ ID NO: 393 - DNA, 35 bp, Artificial Sequence
  Synthetic polynucleotide 12 (R3DV1)
accatgcacgcgatagacacgtacaaaactacaaa

SEQ ID NO: 394 - DNA, 35 bp, Artificial Sequence
  Synthetic polynucleotide 12 (R3DV1)
aatgaacacacgtaggagagctgaacaaagtaggc

SEQ ID NO: 395 - DNA, 25 bp, Artificial Sequence
  Synthetic polynucleotide 12 (R3DV1)
cgcatggagaagtacggcggctggc

SEQ ID NO: 396 - DNA, 31 bp, Artificial Sequence
  Synthetic polynucleotide 12 (R3DV1)
agaatgaaggcgtaagagcactaagcggaga

SEQ ID NO: 397 - DNA, 36 bp, Artificial Sequence
  Synthetic polynucleotide 12 (R3DV1)
tggagacataagcacatgggaacgtcaaaaaatcag

SEQ ID NO: 398 - DNA, 34 bp, Artificial Sequence
  Synthetic polynucleotide 12 (R3DV1)
agagtgcaaacataaacacatgagctcatgcgag

SEQ ID NO: 399 - DNA, 30 bp, Artificial Sequence
  Synthetic polynucleotide 12 (R3DV1)
agtagcacaaatcgagaggtagaggcgtcg

SEQ ID NO: 400 - DNA, 29 bp, Artificial Sequence
  Synthetic polynucleotide 12 (R3DV1)
agggagtagcggagtacccaacatggacc

SEQ ID NO: 401 - DNA, 27 bp, Artificial Sequence
  Synthetic polynucleotide 12 (R3DV1)
actggaaaactcagggcggtggacgga

SEQ ID NO: 402 - DNA, 33 bp, Artificial Sequence
  Synthetic polynucleotide 12 (R3DV1)
tgaggaaggtacacgggtagaaacatagcgcga

SEQ ID NO: 403 - DNA, 43 bp, Artificial Sequence
  Synthetic polynucleotide 12 (R3DV1)
tgagagcaataagaaaggtgaaagcatgagagactacagaaga

SEQ ID NO: 404 - DNA, 31 bp, Artificial Sequence
  Synthetic polynucleotide 12 (R3DV1)
tggacacgataaaaacgtaggcaagctcgag

SEQ ID NO: 405 - DNA, 32 bp, Artificial Sequence
  Synthetic polynucleotide 13 (B4DV1)
agacccactaaggcaatgaaagcctagaggca

SEQ ID NO: 406 - DNA, 26 bp, Artificial Sequence
  Synthetic polynucleotide 13 (B4DV1)
tgacgggataaacgggctcgggaagc

SEQ ID NO: 407 - DNA, 27 bp, Artificial Sequence
  Synthetic polynucleotide 13 (B4DV1)
tgccgggctgacgagaatggaggccta

SEQ ID NO: 408 - DNA, 33 bp, Artificial Sequence
  Synthetic polynucleotide 13 (B4DV1)
aagaaactggagcgatcagacagggtacgacgc

SEQ ID NO: 409 - DNA, 28 bp, Artificial Sequence
  Synthetic polynucleotide 13 (B4DV1)
tcacgcaggatgacagcaatacgacgct

SEQ ID NO: 410 - DNA, 37 bp, Artificial Sequence
  Synthetic polynucleotide 13 (B4DV1)
acaggaatggaaagaatgcgagagctaaaacagtcca

SEQ ID NO: 411 - DNA, 30 bp, Artificial Sequence
  Synthetic polynucleotide 13 (B4DV1)
agggtagagcggtagagctctagggaacta

SEQ ID NO: 412 - DNA, 40 bp, Artificial Sequence
  Synthetic polynucleotide 13 (B4DV1)
agaagagatggagaaatagagacgatagaaacctagcaaa

SEQ ID NO: 413 - DNA, 26 bp, Artificial Sequence
  Synthetic polynucleotide 13 (B4DV1)
atccgaagctcgggagatccagcgag

SEQ ID NO: 414 - DNA, 31 bp, Artificial Sequence
  Synthetic polynucleotide 13 (B4DV1)
tgaggagatacacgaatgcgagcgatggcga

SEQ ID NO: 415 - DNA, 35 bp, Artificial Sequence
  Synthetic polynucleotide 13 (B4DV1)
actgccaacggctgaacacaatgagcaaatggaga

SEQ ID NO: 416 - DNA, 35 bp, Artificial Sequence
  Synthetic polynucleotide 13 (B4DV1)
aataagcgaacatagggcgataagagaccgcggca

SEQ ID NO: 417 - DNA, 33 bp, Artificial Sequence
  Synthetic polynucleotide 13 (B4DV1)
acgtcgggaggtcaacaagtagaggaataaacc

SEQ ID NO: 418 - DNA, 41 bp, Artificial Sequence
  Synthetic polynucleotide 13 (B4DV1)
agtgggaaaatcagaaaataaagaggataaaggcgtcagga

SEQ ID NO: 419 - DNA, 32 bp, Artificial Sequence
  Synthetic polynucleotide 13 (B4DV1)
aatgggaggaatcgggaaatgaacgcgtaaga

SEQ ID NO: 420 - DNA, 31 bp, Artificial Sequence
  Synthetic polynucleotide 13 (B4DV1)
agatagaaaggatgccgaggagtcaaccgaa

SEQ ID NO: 421 - DNA, 40 bp, Artificial Sequence
  Synthetic polynucleotide 13 (B4DV1)
tgacagacgtagggaaagatacaacaatcaccaaatcaaa

SEQ ID NO: 422 - DNA, 29 bp, Artificial Sequence
  Synthetic polynucleotide 13 (B4DV1)
aagtgcgagcagtcgaggaatcgggaggt

SEQ ID NO: 423 - DNA, 38 bp, Artificial Sequence
  Synthetic polynucleotide 13 (B4DV1)
agaaggaatgacaacgatgaagaacatcaaaacgtgac

SEQ ID NO: 424 - DNA, 35 bp, Artificial Sequence
  Synthetic polynucleotide 13 (B4DV1)
accctaaggccctagagcgataaaagagtgaggca

SEQ ID NO: 425 - DNA, 23 bp, Artificial Sequence
  Synthetic polynucleotide 13 (B4DV1)
atcgagggatgcgcgcgtagaca

SEQ ID NO: 426 - DNA, 35 bp, Artificial Sequence
  Synthetic polynucleotide 13 (B4DV1)
agtgcgaacgtagaccactaagaaagtcagcagaa

SEQ ID NO: 427 - DNA, 46 bp, Artificial Sequence
  Synthetic polynucleotide 13 (B4DV1)
taaacagagtaaaccactagcaagatcaaagactaaaaaactacgc

SEQ ID NO: 428 - DNA, 24 bp, Artificial Sequence
  Synthetic polynucleotide 13 (B4DV1)
accgtagccagactcgggcaatga

SEQ ID NO: 429 - DNA, 36 bp, Artificial Sequence
  Synthetic polynucleotide 13 (B4DV1)
acggatcaaaagataagggaaatgacaggatgcaga

SEQ ID NO: 430 - DNA, 34 bp, Artificial Sequence
  Synthetic polynucleotide 13 (B4DV1)
agaataacgggactacaaggctaaggcagtcaga

SEQ ID NO: 431 - DNA, 31 bp, Artificial Sequence
  Synthetic polynucleotide 13 (B4DV1)
aagtaacgcactggcagggtgaagacctgca

SEQ ID NO: 432 - DNA, 27 bp, Artificial Sequence
  Synthetic polynucleotide 13 (B4DV1)
acagtgaaagggtcgggcaatagcaga

SEQ ID NO: 433 - DNA, 35 bp, Artificial Sequence
  Synthetic polynucleotide 13 (B4DV1)
agtgaccacatacgccaatcgacaagtaaagaggt

SEQ ID NO: 434 - DNA, 42 bp, Artificial Sequence
  Synthetic polynucleotide 13 (B4DV1)
aaagcaactaacgaacgtacaagaaataaccggctgaaagga

SEQ ID NO: 435 - DNA, 37 bp, Artificial Sequence
  Synthetic polynucleotide 13 (B4DV1)
actagcgaaatgagggagtgaagaaataagcagaact

SEQ ID NO: 436 - DNA, 34 bp, Artificial Sequence
  Synthetic polynucleotide 13 (B4DV1)
agggagctgcgacgggtggcagagagtcgagaga

SEQ ID NO: 437 - DNA, 40 bp, Artificial Sequence
  Synthetic polynucleotide 13 (B4DV1)
tacaaaggtaaacaaactcgcaagggtgaagagaccatgg

SEQ ID NO: 438 - DNA, 31 bp, Artificial Sequence
  Synthetic polynucleotide 14 (G4DV1)
agacgataagcaaaatagggccgtgagacaa

SEQ ID NO: 439 - DNA, 30 bp, Artificial Sequence
  Synthetic polynucleotide 14 (G4DV1)
atggcgacatgaagcaatacccaagtgaca

SEQ ID NO: 440 - DNA, 36 bp, Artificial Sequence
  Synthetic polynucleotide 14 (G4DV1)
agctagagaggtaacagcatagacaaccctaacggg

SEQ ID NO: 441 - DNA, 33 bp, Artificial Sequence
  Synthetic polynucleotide 14 (G4DV1)
actagcccaaatagaagagtaaacgggtaggga

SEQ ID NO: 442 - DNA, 32 bp, Artificial Sequence
  Synthetic polynucleotide 14 (G4DV1)
actgaggacctgaaaacctgcaaagactgggc

SEQ ID NO: 443 - DNA, 39 bp, Artificial Sequence
  Synthetic polynucleotide 14 (G4DV1)
agggataggaacaataagaagatgaacagatgagcgagc

SEQ ID NO: 444 - DNA, 39 bp, Artificial Sequence
  Synthetic polynucleotide 14 (G4DV1)
tcgggaaatgagaaaataaaaggcgtacgggagtgggac

SEQ ID NO: 445 - DNA, 30 bp, Artificial Sequence
  Synthetic polynucleotide 14 (G4DV1)
agtcacgagaataaaggcgtcggcagatca

SEQ ID NO: 446 - DNA, 28 bp, Artificial Sequence
  Synthetic polynucleotide 14 (G4DV1)
acggatgcagggcatgggacagtacggg

SEQ ID NO: 447 - DNA, 28 bp, Artificial Sequence
  Synthetic polynucleotide 14 (G4DV1)
agagtacgcggctgaagagctgacaccc

SEQ ID NO: 448 - DNA, 34 bp, Artificial Sequence
  Synthetic polynucleotide 14 (G4DV1)
tgagggaagtaggcaaataaaagggtagcccact

SEQ ID NO: 449 - DNA, 25 bp, Artificial Sequence
  Synthetic polynucleotide 14 (G4DV1)
agcgagcgtcaccgggtggaaagct

SEQ ID NO: 450 - DNA, 31 bp, Artificial Sequence
  Synthetic polynucleotide 14 (G4DV1)
aggaacgtcggaaactaggagagtcagcagc

SEQ ID NO: 451 - DNA, 32 bp, Artificial Sequence
  Synthetic polynucleotide 14 (G4DV1)
tccgaagctgagaaactcggcagatcacggac

SEQ ID NO: 452 - DNA, 30 bp, Artificial Sequence
  Synthetic polynucleotide 14 (G4DV1)
cgcgggaagatgaaaagctgaggagggtgg

SEQ ID NO: 453 - DNA, 30 bp, Artificial Sequence
  Synthetic polynucleotide 14 (G4DV1)
acgggtcagaacgtgggaaactagacgacg

SEQ ID NO: 454 - DNA, 33 bp, Artificial Sequence
  Synthetic polynucleotide 14 (G4DV1)
tgggcgaatacgcacgtaaaggagtacgacaca

SEQ ID NO: 455 - DNA, 31 bp, Artificial Sequence
  Synthetic polynucleotide 14 (G4DV1)
tcagggcctgggaagataccaagatgccgga

SEQ ID NO: 456 - DNA, 38 bp, Artificial Sequence
  Synthetic polynucleotide 14 (G4DV1)
agatcgaaaactaaagcagtggaacagtcaacaaatca

SEQ ID NO: 457 - DNA, 31 bp, Artificial Sequence
  Synthetic polynucleotide 14 (G4DV1)
agggcgataagcgaataaggaggtcagcagg

SEQ ID NO: 458 - DNA, 28 bp, Artificial Sequence
  Synthetic polynucleotide 14 (G4DV1)
tggaaaccgctaagaccgtgggaaactc

SEQ ID NO: 459 - DNA, 28 bp, Artificial Sequence
  Synthetic polynucleotide 14 (G4DV1)
aggaaatacgggcagtaaggcggctggc

SEQ ID NO: 460 - DNA, 27 bp, Artificial Sequence
  Synthetic polynucleotide 14 (G4DV1)
agactagcgacatgagcgggtcacaga

SEQ ID NO: 461 - DNA, 33 bp, Artificial Sequence
  Synthetic polynucleotide 14 (G4DV1)
aggtaacgcaataacaaaatcgcgcagtggcac

SEQ ID NO: 462 - DNA, 26 bp, Artificial Sequence
  Synthetic polynucleotide 14 (G4DV1)
agtaaaggcctcgggaagtgcggaga

SEQ ID NO: 463 - DNA, 31 bp, Artificial Sequence
  Synthetic polynucleotide 14 (G4DV1)
tgcaagagtaccaaaggctaaggcactgggc

SEQ ID NO: 464 - DNA, 30 bp, Artificial Sequence
  Synthetic polynucleotide 14 (G4DV1)
acgatacgggaactagggcgactgacagca

SEQ ID NO: 465 - DNA, 25 bp, Artificial Sequence
  Synthetic polynucleotide 14 (G4DV1)
tccacgcagtaagagaatggcggga

SEQ ID NO: 466 - DNA, 33 bp, Artificial Sequence
  Synthetic polynucleotide 14 (G4DV1)
tggagcgctaagacggtgaaccaataagggcct

SEQ ID NO: 467 - DNA, 34 bp, Artificial Sequence
  Synthetic polynucleotide 14 (G4DV1)
aacaggatgacaaggatgcgggaatgagccacta

SEQ ID NO: 468 - DNA, 29 bp, Artificial Sequence
  Synthetic polynucleotide 14 (G4DV1)
aaggaagtagaggagtaagccgggtgcga

SEQ ID NO: 469 - DNA, 32 bp, Artificial Sequence
  Synthetic polynucleotide 14 (G4DV1)
agctggagggaataggaaaatacgaggatggg

SEQ ID NO: 470 - DNA, 29 bp, Artificial Sequence
  Synthetic polynucleotide 14 (G4DV1)
caggtgaggagaaatcggacggatgaacg

SEQ ID NO: 471 - DNA, 27 bp, Artificial Sequence
  Synthetic polynucleotide 14 (G4DV1)
gctggcaaggtaaaagggtagcggaat

SEQ ID NO: 472 - DNA, 26 bp, Artificial Sequence
  Synthetic polynucleotide 14 (G4DV1)
agagagcagtccgcaggtcaaacggg

SEQ ID NO: 473 - DNA, 24 bp, Artificial Sequence
  Synthetic polynucleotide 14 (G4DV1)
tgcacgggctcagacgggccatgg

SEQ ID NO: 474 - DNA, 37 bp, Artificial Sequence
  Synthetic polynucleotide 15 (Y4DV1)
agaaaaatggcaagctaagaggaatcaagaactgccc

SEQ ID NO: 475 - DNA, 34 bp, Artificial Sequence
  Synthetic polynucleotide 15 (Y4DV1)
acctaagaccaatgaggcgataaccgaaatcggg

SEQ ID NO: 476 - DNA, 33 bp, Artificial Sequence
  Synthetic polynucleotide 15 (Y4DV1)
caatagccgaataaagggaatgagacgggtgcg

SEQ ID NO: 477 - DNA, 41 bp, Artificial Sequence
  Synthetic polynucleotide 15 (Y4DV1)
cgactacacaagtgcgcaactaaaaaaataacgaagtggga

SEQ ID NO: 478 - DNA, 26 bp, Artificial Sequence
  Synthetic polynucleotide 15 (Y4DV1)
agcgctacaaagatgggcagatcggc

SEQ ID NO: 479 - DNA, 32 bp, Artificial Sequence
  Synthetic polynucleotide 15 (Y4DV1)
aggtaaggacgtagggaagtacaggagctccg

SEQ ID NO: 480 - DNA, 29 bp, Artificial Sequence
  Synthetic polynucleotide 15 (Y4DV1)
cgactaggaccatccaacactggcaggat

SEQ ID NO: 481 - DNA, 30 bp, Artificial Sequence
  Synthetic polynucleotide 15 (Y4DV1)
agagcggtgaaaagctggagaaacgtcggg

SEQ ID NO: 482 - DNA, 25 bp, Artificial Sequence
  Synthetic polynucleotide 15 (Y4DV1)
acgtgggcaggtcagagggtgcaga

SEQ ID NO: 483 - DNA, 31 bp, Artificial Sequence
  Synthetic polynucleotide 15 (Y4DV1)
agtgacgggcataagcacatacaacggtagc

SEQ ID NO: 484 - DNA, 39 bp, Artificial Sequence
  Synthetic polynucleotide 15 (Y4DV1)
agagtcgaaaacataaagagactggacgaatcagagact

SEQ ID NO: 485 - DNA, 24 bp, Artificial Sequence
  Synthetic polynucleotide 15 (Y4DV1)
agcggactagccacctgggagagt

SEQ ID NO: 486 - DNA, 32 bp, Artificial Sequence
  Synthetic polynucleotide 15 (Y4DV1)
acccgggatacaagggataagaggaataggcg

SEQ ID NO: 487 - DNA, 33 bp, Artificial Sequence
  Synthetic polynucleotide 15 (Y4DV1)
agtggacagatggaagcatgggaggatcacaga

SEQ ID NO: 488 - DNA, 32 bp, Artificial Sequence
  Synthetic polynucleotide 15 (Y4DV1)
actagggagatagcgagataccagcgtggaga

SEQ ID NO: 489 - DNA, 43 bp, Artificial Sequence
  Synthetic polynucleotide 15 (Y4DV1)
agtaaaaaaatgagggactaagggaatgaaaaagtaagaaacc

SEQ ID NO: 490 - DNA, 24 bp, Artificial Sequence
  Synthetic polynucleotide 15 (Y4DV1)
cgcggggtacaccagtgcagcagt

SEQ ID NO: 491 - DNA, 33 bp, Artificial Sequence
  Synthetic polynucleotide 15 (Y4DV1)
aggagaatacacgaatgcagccagtcaagagaa

SEQ ID NO: 492 - DNA, 39 bp, Artificial Sequence
  Synthetic polynucleotide 15 (Y4DV1)
atgaagaactaagagagtgcgagagtacagagctacgca

SEQ ID NO: 493 - DNA, 30 bp, Artificial Sequence
  Synthetic polynucleotide 15 (Y4DV1)
agtgcccaagtgagagaatagcgggcctca

SEQ ID NO: 494 - DNA, 32 bp, Artificial Sequence
  Synthetic polynucleotide 15 (Y4DV1)
agcgatcaacgacataacaggagtcaggagaa

SEQ ID NO: 495 - DNA, 28 bp, Artificial Sequence
  Synthetic polynucleotide 15 (Y4DV1)
tcgcaaagtcacgggatgcgagcagtgg

SEQ ID NO: 496 - DNA, 38 bp, Artificial Sequence
  Synthetic polynucleotide 15 (Y4DV1)
acaaatcagcaaaatcaaagactcacaagatccgacaa

SEQ ID NO: 497 - DNA, 38 bp, Artificial Sequence
  Synthetic polynucleotide 15 (Y4DV1)
tagagggataaacaagatacaaacatccagagactggc

SEQ ID NO: 498 - DNA, 36 bp, Artificial Sequence
  Synthetic polynucleotide 15 (Y4DV1)
aggataaagaaagtacaaagcgtgccaagctaagga

SEQ ID NO: 499 - DNA, 37 bp, Artificial Sequence
  Synthetic polynucleotide 15 (Y4DV1)
agtagagaaatccaagaatacagaggtgacgccgtga

SEQ ID NO: 500 - DNA, 26 bp, Artificial Sequence
  Synthetic polynucleotide 15 (Y4DV1)
agacatgcgcaggtgagcaggatagg

SEQ ID NO: 501 - DNA, 29 bp, Artificial Sequence
  Synthetic polynucleotide 15 (Y4DV1)
agactaaaggcgtacgggaatgcgaaact

SEQ ID NO: 502 - DNA, 28 bp, Artificial Sequence
  Synthetic polynucleotide 15 (Y4DV1)
agaaggctgaaaggatcgacccactcgc

SEQ ID NO: 503 - DNA, 37 bp, Artificial Sequence
  Synthetic polynucleotide 15 (Y4DV1)
agcgtagagggctacgacaactaaagacataagcaga

SEQ ID NO: 504 - DNA, 26 bp, Artificial Sequence
  Synthetic polynucleotide 15 (Y4DV1)
tgaagcccatcaaggacatggcgcga

SEQ ID NO: 505 - DNA, 38 bp, Artificial Sequence
  Synthetic polynucleotide 15 (Y4DV1)
tgggaagatccaagagatccaagccctaggaaagatga

SEQ ID NO: 506 - DNA, 27 bp, Artificial Sequence
  Synthetic polynucleotide 15 (Y4DV1)
acgcctgaacagctaagagcgggtcca

SEQ ID NO: 507 - DNA, 34 bp, Artificial Sequence
  Synthetic polynucleotide 15 (Y4DV1)
aggataagaacatgcagaaatggacgagccatgg

SEQ ID NO: 508 - DNA, 39 bp, Artificial Sequence
  Synthetic polynucleotide 16 (R4DV1)
agaccaaaagtcaagaaagtaccgggctagaagagctga

SEQ ID NO: 509 - DNA, 35 bp, Artificial Sequence
  Synthetic polynucleotide 16 (R4DV1)
agccataagcgagtagcagaatagaaagatcccaa

SEQ ID NO: 510 - DNA, 32 bp, Artificial Sequence
  Synthetic polynucleotide 16 (R4DV1)
aagtcccagggatagacgagtaggaaggtgaa

SEQ ID NO: 511 - DNA, 29 bp, Artificial Sequence
  Synthetic polynucleotide 16 (R4DV1)
aaaatggcgaggtagcgacatgcaaaggt

SEQ ID NO: 512 - DNA, 36 bp, Artificial Sequence
  Synthetic polynucleotide 16 (R4DV1)
aaaacgatgaaaaactacgagggtggaagaataagc

SEQ ID NO: 513 - DNA, 32 bp, Artificial Sequence
  Synthetic polynucleotide 16 (R4DV1)
aggtgacgaagtaaacgggtcaaaccgagctc

SEQ ID NO: 514 - DNA, 32 bp, Artificial Sequence
  Synthetic polynucleotide 16 (R4DV1)
agatcgaacgataggaaacatgaccgggtcac

SEQ ID NO: 515 - DNA, 34 bp, Artificial Sequence
  Synthetic polynucleotide 16 (R4DV1)
acgatcgagaggtcccaaaggatagaagaagtga

SEQ ID NO: 516 - DNA, 33 bp, Artificial Sequence
  Synthetic polynucleotide 16 (R4DV1)
aaaggtgagcaaggtggcgaaaataaaaagata

SEQ ID NO: 517 - DNA, 35 bp, Artificial Sequence
  Synthetic polynucleotide 16 (R4DV1)
aaagaataagacagtagcgggaatacgacactaga

SEQ ID NO: 518 - DNA, 28 bp, Artificial Sequence
  Synthetic polynucleotide 16 (R4DV1)
aggatcgggacatgcagcagtaaaccaa

SEQ ID NO: 519 - DNA, 35 bp, Artificial Sequence
  Synthetic polynucleotide 16 (R4DV1)
taggaggataacagggcatggaagagtgggacggt

SEQ ID NO: 520 - DNA, 32 bp, Artificial Sequence
  Synthetic polynucleotide 16 (R4DV1)
aagaccctacacgaatacaagcagtgccagga

SEQ ID NO: 521 - DNA, 29 bp, Artificial Sequence
  Synthetic polynucleotide 16 (R4DV1)
tggcgcgagtgacaaaaagtagaagggtg

SEQ ID NO: 522 - DNA, 36 bp, Artificial Sequence
  Synthetic polynucleotide 16 (R4DV1)
accgagtcgagagatagagacgtaaggaagtaggga

SEQ ID NO: 523 - DNA, 28 bp, Artificial Sequence
  Synthetic polynucleotide 16 (R4DV1)
aggtgggacgatcgaaagatcgaagagt

SEQ ID NO: 524 - DNA, 31 bp, Artificial Sequence
  Synthetic polynucleotide 16 (R4DV1)
aggaggcgtcgaaaaatccggaaaataggga

SEQ ID NO: 525 - DNA, 29 bp, Artificial Sequence
  Synthetic polynucleotide 16 (R4DV1)
agatggacggatcgggacggtgaggagga

SEQ ID NO: 526 - DNA, 29 bp, Artificial Sequence
  Synthetic polynucleotide 16 (R4DV1)
atagcaaaagtcccgcgggtacgaaaggt

SEQ ID NO: 527 - DNA, 27 bp, Artificial Sequence
  Synthetic polynucleotide 16 (R4DV1)
cgggaaggtcaagcaatcaggcgctca

SEQ ID NO: 528 - DNA, 36 bp, Artificial Sequence
  Synthetic polynucleotide 16 (R4DV1)
acgggactgaacaaataaggacatacacaagtcggc

SEQ ID NO: 529 - DNA, 29 bp, Artificial Sequence
  Synthetic polynucleotide 16 (R4DV1)
acgtcacgaactcaaaaggtggagaaggt

SEQ ID NO: 530 - DNA, 31 bp, Artificial Sequence
  Synthetic polynucleotide 16 (R4DV1)
agcgaaatcgaggagtggagaaggtaaagaa

SEQ ID NO: 531 - DNA, 32 bp, Artificial Sequence
  Synthetic polynucleotide 16 (R4DV1)
atgggaaggctaaagaaatggcagggtagaga

SEQ ID NO: 532 - DNA, 35 bp, Artificial Sequence
  Synthetic polynucleotide 16 (R4DV1)
actgggacggtaaacgcatgaaagaatcagggagt

SEQ ID NO: 533 - DNA, 36 bp, Artificial Sequence
  Synthetic polynucleotide 16 (R4DV1)
agaagaacgtgaagggataggagaactcaacagggt

SEQ ID NO: 534 - DNA, 34 bp, Artificial Sequence
  Synthetic polynucleotide 16 (R4DV1)
agcagaagtggaaagcatggcaagaatggcagca

SEQ ID NO: 535 - DNA, 32 bp, Artificial Sequence
  Synthetic polynucleotide 16 (R4DV1)
tgaaaagatccaggagtaagcgagctgaagaa

SEQ ID NO: 536 - DNA, 39 bp, Artificial Sequence
  Synthetic polynucleotide 16 (R4DV1)
atggagacgtaacaacatagcgggagtaggcgcgtgaca

SEQ ID NO: 537 - DNA, 28 bp, Artificial Sequence
  Synthetic polynucleotide 16 (R4DV1)
agataacgcgaatgcggaggtcgaggaa

SEQ ID NO: 538 - DNA, 28 bp, Artificial Sequence
  Synthetic polynucleotide 16 (R4DV1)
tccgcaagtgaacacgtcaacgcaatga

SEQ ID NO: 539 - DNA, 34 bp, Artificial Sequence
  Synthetic polynucleotide 16 (R4DV1)
acggatgaacacatgcacgaagtcgacaagtaaa

SEQ ID NO: 540 - DNA, 30 bp, Artificial Sequence
  Synthetic polynucleotide 16 (R4DV1)
aaacgtggaagccatgacaacataacggga

SEQ ID NO: 541 - DNA, 38 bp, Artificial Sequence
  Synthetic polynucleotide 16 (R4DV1)
tggcaggataagagagtagaacgatgcacgagccatgg

SEQ ID NO: 542 - DNA, 30 bp, Artificial Sequence
  Synthetic polynucleotide 17 (B5DV1)
agacagcgatcagagggtaaaacgggatga

SEQ ID NO: 543 - DNA, 33 bp, Artificial Sequence
  Synthetic polynucleotide 17 (B5DV1)
agcagtgaaaggacctcagcgaatgaaaaacga

SEQ ID NO: 544 - DNA, 39 bp, Artificial Sequence
  Synthetic polynucleotide 17 (B5DV1)
tggccagatccaaagataaaaaactgaaagactacggaa

SEQ ID NO: 545 - DNA, 43 bp, Artificial Sequence
  Synthetic polynucleotide 17 (B5DV1)
atacaagaatagaagggtaaacgactgagaaagtacgaagcct

SEQ ID NO: 546 - DNA, 34 bp, Artificial Sequence
  Synthetic polynucleotide 17 (B5DV1)
agacgggtaaaaaaggtcgggaagggtaacgcca

SEQ ID NO: 547 - DNA, 41 bp, Artificial Sequence
  Synthetic polynucleotide 17 (B5DV1)
tagacaaatgagaaggtaaaggcatggaaaaaatggaggca

SEQ ID NO: 548 - DNA, 26 bp, Artificial Sequence
  Synthetic polynucleotide 17 (B5DV1)
tcgacgaatgcccggctcaaaggata

SEQ ID NO: 549 - DNA, 29 bp, Artificial Sequence
  Synthetic polynucleotide 17 (B5DV1)
acggactagcgcggtaaaagggaatgcgg

SEQ ID NO: 550 - DNA, 27 bp, Artificial Sequence
  Synthetic polynucleotide 17 (B5DV1)
acgatcgaagaagctccggaccgatcc

SEQ ID NO: 551 - DNA, 30 bp, Artificial Sequence
  Synthetic polynucleotide 17 (B5DV1)
acggaatagagacatacgacagtgcgccaa

SEQ ID NO: 552 - DNA, 37 bp, Artificial Sequence
  Synthetic polynucleotide 17 (B5DV1)
atggaccgataaaagggtagacgaaataacaggatga

SEQ ID NO: 553 - DNA, 38 bp, Artificial Sequence
  Synthetic polynucleotide 17 (B5DV1)
acaggactcggagaaataacaaagtggagaaagtacaa

SEQ ID NO: 554 - DNA, 34 bp, Artificial Sequence
  Synthetic polynucleotide 17 (B5DV1)
aagtcaacgaataggccagtggcaaaagtgagcg

SEQ ID NO: 555 - DNA, 35 bp, Artificial Sequence
  Synthetic polynucleotide 17 (B5DV1)
agtgaacaggtagaggagtggaaaagtacaaagga

SEQ ID NO: 556 - DNA, 41 bp, Artificial Sequence
  Synthetic polynucleotide 17 (B5DV1)
tgcaaaaatgaaaaggtagaaaactaaggcagtacaggcat

SEQ ID NO: 557 - DNA, 41 bp, Artificial Sequence
  Synthetic polynucleotide 17 (B5DV1)
aaacgactcacaaactagaaaactacagcagatcgaagcat

SEQ ID NO: 558 - DNA, 43 bp, Artificial Sequence
  Synthetic polynucleotide 17 (B5DV1)
aaaggaaataggagagaataaaaacgcctaacaaactacaaga

SEQ ID NO: 559 - DNA, 27 bp, Artificial Sequence
  Synthetic polynucleotide 17 (B5DV1)
actacgcggagtgcgagacgtcaggca

SEQ ID NO: 560 - DNA, 32 bp, Artificial Sequence
  Synthetic polynucleotide 17 (B5DV1)
agtgaggacctgaaacaatgcaagaatggcga

SEQ ID NO: 561 - DNA, 27 bp, Artificial Sequence
  Synthetic polynucleotide 17 (B5DV1)
agtggacgcggtagcgggataagcaaa

SEQ ID NO: 562 - DNA, 34 bp, Artificial Sequence
  Synthetic polynucleotide 17 (B5DV1)
tacaccggtagatatcataggaaggtcacgcaaa

SEQ ID NO: 563 - DNA, 28 bp, Artificial Sequence
  Synthetic polynucleotide 17 (B5DV1)
tggaggagtcaagaaactggccaagtga

SEQ ID NO: 564 - DNA, 30 bp, Artificial Sequence
  Synthetic polynucleotide 17 (B5DV1)
agccctcggcagatacgcaaagtacgacaa

SEQ ID NO: 565 - DNA, 34 bp, Artificial Sequence
  Synthetic polynucleotide 17 (B5DV1)
ataagaggctcagaagatccagacgagtgcagga

SEQ ID NO: 566 - DNA, 36 bp, Artificial Sequence
  Synthetic polynucleotide 17 (B5DV1)
ataagacaatcaagagaatgaacgcatcggaacact

SEQ ID NO: 567 - DNA, 27 bp, Artificial Sequence
  Synthetic polynucleotide 17 (B5DV1)
aggcagcagtgggaccggtaaaagcat

SEQ ID NO: 568 - DNA, 26 bp, Artificial Sequence
  Synthetic polynucleotide 17 (B5DV1)
agctagctcgggcgatggaggcacgt

SEQ ID NO: 569 - DNA, 35 bp, Artificial Sequence
  Synthetic polynucleotide 17 (B5DV1)
acaaaggtgacaaaagtaacgggaatacccaccgt

SEQ ID NO: 570 - DNA, 33 bp, Artificial Sequence
  Synthetic polynucleotide 17 (B5DV1)
accaggatgaccagggatcgcgaagatagcgga

SEQ ID NO: 571 - DNA, 27 bp, Artificial Sequence
  Synthetic polynucleotide 17 (B5DV1)
atcgagccctcaggagctaggccagca

SEQ ID NO: 572 - DNA, 36 bp, Artificial Sequence
  Synthetic polynucleotide 17 (B5DV1)
tagagacgtcacgagatgaagggataaaggaagtca

SEQ ID NO: 573 - DNA, 31 bp, Artificial Sequence
  Synthetic polynucleotide 17 (B5DV1)
aagaatgggagaactccggacgtacgaggct

SEQ ID NO: 574 - DNA, 39 bp, Artificial Sequence
  Synthetic polynucleotide 17 (B5DV1)
acagaggtgaaaagataaagcagggtgaggaaggcatgc

SEQ ID NO: 575 - DNA, 38 bp, Artificial Sequence
  Synthetic polynucleotide 18 (G5DV1)
agacaagagatagaaagcagtgaaagaataggacggtc

SEQ ID NO: 576 - DNA, 27 bp, Artificial Sequence
  Synthetic polynucleotide 18 (G5DV1)
agaggatggagggcctccgggcgtggc

SEQ ID NO: 577 - DNA, 29 bp, Artificial Sequence
  Synthetic polynucleotide 18 (G5DV1)
acgactaggacgatgcggaaataacgaca

SEQ ID NO: 578 - DNA, 29 bp, Artificial Sequence
  Synthetic polynucleotide 18 (G5DV1)
agtggaaacctagccagctaaggaagcta

SEQ ID NO: 579 - DNA, 31 bp, Artificial Sequence
  Synthetic polynucleotide 18 (G5DV1)
agggcaatggcaaagtaaggaagtacggaaa

SEQ ID NO: 580 - DNA, 33 bp, Artificial Sequence
  Synthetic polynucleotide 18 (G5DV1)
taggaccatagaagactggaccgatacagcgct

SEQ ID NO: 581 - DNA, 28 bp, Artificial Sequence
  Synthetic polynucleotide 18 (G5DV1)
agggcgggtaaacgagtgaaagggtgga

SEQ ID NO: 582 - DNA, 33 bp, Artificial Sequence
  Synthetic polynucleotide 18 (G5DV1)
acaataaggacagtgcagcaggtaagaccacta

SEQ ID NO: 583 - DNA, 32 bp, Artificial Sequence
  Synthetic polynucleotide 18 (G5DV1)
aaagactccacgacgtacagagactccgcgcc

SEQ ID NO: 584 - DNA, 26 bp, Artificial Sequence
  Synthetic polynucleotide 18 (G5DV1)
tggaacgatcgaagcgtaacgggcat

SEQ ID NO: 585 - DNA, 36 bp, Artificial Sequence
  Synthetic polynucleotide 18 (G5DV1)
aaggaaaatagagaaggtcgaggaaataaagggaaa

SEQ ID NO: 586 - DNA, 35 bp, Artificial Sequence
  Synthetic polynucleotide 18 (G5DV1)
tggagaaactaagcggatagggaaataaacgaacc

SEQ ID NO: 587 - DNA, 29 bp, Artificial Sequence
  Synthetic polynucleotide 18 (G5DV1)
tcagggaatcccaaagtccgaccaatgac

SEQ ID NO: 588 - DNA, 28 bp, Artificial Sequence
  Synthetic polynucleotide 18 (G5DV1)
agactgcacgcatccgagcgtaaaaaca

SEQ ID NO: 589 - DNA, 31 bp, Artificial Sequence
  Synthetic polynucleotide 18 (G5DV1)
tgagaccaataacgagatcggcaagtcgaga

SEQ ID NO: 590 - DNA, 39 bp, Artificial Sequence
  Synthetic polynucleotide 18 (G5DV1)
agtcgcagaatcaaacaacctgagaacctgcgggagtga

SEQ ID NO: 591 - DNA, 36 bp, Artificial Sequence
  Synthetic polynucleotide 18 (G5DV1)
acggataagacggtaagagaaataagagcatgagaa

SEQ ID NO: 592 - DNA, 36 bp, Artificial Sequence
  Synthetic polynucleotide 18 (G5DV1)
actgggacgatagaacgatagccaagtaaaagggta

SEQ ID NO: 593 - DNA, 33 bp, Artificial Sequence
  Synthetic polynucleotide 18 (G5DV1)
agagaatggaacgaagtgcaaaaagtggcagaa

SEQ ID NO: 594 - DNA, 40 bp, Artificial Sequence
  Synthetic polynucleotide 18 (G5DV1)
tgaacagataggcagatcagaagaatgaggaagtcgcaga

SEQ ID NO: 595 - DNA, 34 bp, Artificial Sequence
  Synthetic polynucleotide 18 (G5DV1)
aataagagggtgggagcgatgccgggatgcgcga

SEQ ID NO: 596 - DNA, 39 bp, Artificial Sequence
  Synthetic polynucleotide 18 (G5DV1)
atgcgaaggtaagagaatgcaggagtaaagaggactgaa

SEQ ID NO: 597 - DNA, 36 bp, Artificial Sequence
  Synthetic polynucleotide 18 (G5DV1)
aagatcgggacatgaaacgataggaaggtacggcga

SEQ ID NO: 598 - DNA, 40 bp, Artificial Sequence
  Synthetic polynucleotide 18 (G5DV1)
tgagacagtacaaaagtgaaagggtgacagcctgcgcgga

SEQ ID NO: 599 - DNA, 39 bp, Artificial Sequence
  Synthetic polynucleotide 18 (G5DV1)
tatcagggatgccacgatgggcacatgcccaaaatgaaa

SEQ ID NO: 600 - DNA, 35 bp, Artificial Sequence
  Synthetic polynucleotide 18 (G5DV1)
aaatcaccaaataccaaaatgaagccgatgcggga

SEQ ID NO: 601 - DNA, 34 bp, Artificial Sequence
  Synthetic polynucleotide 18 (G5DV1)
atgcgctagctaacgagcataaaacggtaggaaa

SEQ ID NO: 602 - DNA, 35 bp, Artificial Sequence
  Synthetic polynucleotide 18 (G5DV1)
atggaaagctaaccgcagtggaagaataaggagct

SEQ ID NO: 603 - DNA, 33 bp, Artificial Sequence
  Synthetic polynucleotide 18 (G5DV1)
acgcaaatacgccgaataaggaagtagcggact

SEQ ID NO: 604 - DNA, 38 bp, Artificial Sequence
  Synthetic polynucleotide 18 (G5DV1)
aaggaggtagacgaataggcgaataacgcgagtcgaga

SEQ ID NO: 605 - DNA, 40 bp, Artificial Sequence
  Synthetic polynucleotide 18 (G5DV1)
aatccaagactacaggactcagcagatgaaaaaactagaa

SEQ ID NO: 606 - DNA, 50 bp, Artificial Sequence
  Synthetic polynucleotide 18 (G5DV1)
acgtgaaggcctaagaaacataagacactgaaagagtagcggaggcatgc

SEQ ID NO: 607 - DNA, 39 bp, Artificial Sequence
  Synthetic polynucleotide 19 (Y5DV1)
agagagtaaggaaatgagaacagtgaagacatcccaaga

SEQ ID NO: 608 - DNA, 35 bp, Artificial Sequence
  Synthetic polynucleotide 19 (Y5DV1)
aatgaaaaaagtggagaggtcgaacggtagagcag

SEQ ID NO: 609 - DNA, 27 bp, Artificial Sequence
  Synthetic polynucleotide 19 (Y5DV1)
tggagaaggatacgccgatcgccggga

SEQ ID NO: 610 - DNA, 34 bp, Artificial Sequence
  Synthetic polynucleotide 19 (Y5DV1)
taaccgggctaaacacaatgaaacacgtggccga

SEQ ID NO: 611 - DNA, 30 bp, Artificial Sequence
  Synthetic polynucleotide 19 (Y5DV1)
atacggaggaatcagaggaggtggcaggac

SEQ ID NO: 612 - DNA, 34 bp, Artificial Sequence
  Synthetic polynucleotide 19 (Y5DV1)
tgaacgagtgggcgggatagaaaaactacagcga

SEQ ID NO: 613 - DNA, 34 bp, Artificial Sequence
  Synthetic polynucleotide 19 (Y5DV1)
tacgcgcgataggaacctacgagaactaagagga

SEQ ID NO: 614 - DNA, 35 bp, Artificial Sequence
  Synthetic polynucleotide 19 (Y5DV1)
taaagacataagggcctacgcacgagtaaaagagt

SEQ ID NO: 615 - DNA, 39 bp, Artificial Sequence
  Synthetic polynucleotide 19 (Y5DV1)
accgacgtcagacaatagaagggtaaaaagatgaaccga

SEQ ID NO: 616 - DNA, 27 bp, Artificial Sequence
  Synthetic polynucleotide 19 (Y5DV1)
tgagcaaaatccaggcgtcgcaaggtc

SEQ ID NO: 617 - DNA, 35 bp, Artificial Sequence
  Synthetic polynucleotide 19 (Y5DV1)
acgcagtcaagacataagagaatgccagaagtaca

SEQ ID NO: 618 - DNA, 27 bp, Artificial Sequence
  Synthetic polynucleotide 19 (Y5DV1)
agcctgggacggctgagagagatcggg

SEQ ID NO: 619 - DNA, 29 bp, Artificial Sequence
  Synthetic polynucleotide 19 (Y5DV1)
cagtcaaaagggtcaggacatagcgggat

SEQ ID NO: 620 - DNA, 31 bp, Artificial Sequence
  Synthetic polynucleotide 19 (Y5DV1)
agccgaatgcaaagatacgacggtgcaagaa

SEQ ID NO: 621 - DNA, 32 bp, Artificial Sequence
  Synthetic polynucleotide 19 (Y5DV1)
tcacggcataggcaagtgcaaaacgtaacaac

SEQ ID NO: 622 - DNA, 31 bp, Artificial Sequence
  Synthetic polynucleotide 19 (Y5DV1)
actggccaaaatggaagactgaacgcatgac

SEQ ID NO: 623 - DNA, 25 bp, Artificial Sequence
  Synthetic polynucleotide 19 (Y5DV1)
acgggtcacgcagtgcagacctgca

SEQ ID NO: 624 - DNA, 41 bp, Artificial Sequence
  Synthetic polynucleotide 19 (Y5DV1)
acagtacagaaatggaaaactagaagagtaagcaaatcgaa

SEQ ID NO: 625 - DNA, 30 bp, Artificial Sequence
  Synthetic polynucleotide 19 (Y5DV1)
acctccaagggtggaaggatggacaggtga

SEQ ID NO: 626 - DNA, 31 bp, Artificial Sequence
  Synthetic polynucleotide 19 (Y5DV1)
acaggtaaagagatcgcggacatgagaaggt

SEQ ID NO: 627 - DNA, 33 bp, Artificial Sequence
  Synthetic polynucleotide 19 (Y5DV1)
acaaagctaaacaagtcgggaggtgaacgaata

SEQ ID NO: 628 - DNA, 26 bp, Artificial Sequence
  Synthetic polynucleotide 19 (Y5DV1)
aggacgggtaagggacctggaccgga

SEQ ID NO: 629 - DNA, 33 bp, Artificial Sequence
  Synthetic polynucleotide 19 (Y5DV1)
atggcaacatgcaaacataagagggtcaaccaa

SEQ ID NO: 630 - DNA, 37 bp, Artificial Sequence
  Synthetic polynucleotide 19 (Y5DV1)
tggaaggctgaaaagatcgaaaaatgggcgaatacaa

SEQ ID NO: 631 - DNA, 36 bp, Artificial Sequence
  Synthetic polynucleotide 19 (Y5DV1)
aaggtaaagggatagcgggatcagaaggtgggacga

SEQ ID NO: 632 - DNA, 40 bp, Artificial Sequence
  Synthetic polynucleotide 19 (Y5DV1)
tgaaagaatgaagaaataccaagcgataacacgatccgga

SEQ ID NO: 633 - DNA, 27 bp, Artificial Sequence
  Synthetic polynucleotide 19 (Y5DV1)
actgcgggagctgaacaagtcaccgct

SEQ ID NO: 634 - DNA, 26 bp, Artificial Sequence
  Synthetic polynucleotide 19 (Y5DV1)
agccagagggtgcaggggatatcaaa

SEQ ID NO: 635 - DNA, 33 bp, Artificial Sequence
  Synthetic polynucleotide 19 (Y5DV1)
tgggcagatacggagcgataaaaacatgaaagg

SEQ ID NO: 636 - DNA, 28 bp, Artificial Sequence
  Synthetic polynucleotide 19 (Y5DV1)
agtgggccactggaaggatcagcacgta

SEQ ID NO: 637 - DNA, 31 bp, Artificial Sequence
  Synthetic polynucleotide 19 (Y5DV1)
acggcctaaaggactaagagcacatgagcga

SEQ ID NO: 638 - DNA, 42 bp, Artificial Sequence
  Synthetic polynucleotide 19 (Y5DV1)
agtgaagagagtgaagaaactaagaaagagtagagagatgcg

SEQ ID NO: 639 - DNA, 32 bp, Artificial Sequence
  Synthetic polynucleotide 19 (Y5DV1)
agagatagcgaaaatcgacaacatcgcgggag

SEQ ID NO: 640 - DNA, 31 bp, Artificial Sequence
  Synthetic polynucleotide 19 (Y5DV1)
tggaaaactgacgggatgacgagaagcatgc

SEQ ID NO: 641 - DNA, 44 bp, Artificial Sequence
  Synthetic polynucleotide 20 (R5DV1)
agaacagactaaaagaatcaacagatagaggaatgaggaagtgc

SEQ ID NO: 642 - DNA, 39 bp, Artificial Sequence
  Synthetic polynucleotide 20 (R5DV1)
aggaagtaaggaaaactggaagagtaacacaatgggaga

SEQ ID NO: 643 - DNA, 42 bp, Artificial Sequence
  Synthetic polynucleotide 20 (R5DV1)
atacaaaagtcaaccagatggacagatagagaaatgacgaga

SEQ ID NO: 644 - DNA, 33 bp, Artificial Sequence
  Synthetic polynucleotide 20 (R5DV1)
tggaaacagtcacacgctaagggaatggacgcg

SEQ ID NO: 645 - DNA, 27 bp, Artificial Sequence
  Synthetic polynucleotide 20 (R5DV1)
tgaccagatcgggaagatccgggcaat

SEQ ID NO: 646 - DNA, 37 bp, Artificial Sequence
  Synthetic polynucleotide 20 (R5DV1)
aggacagtagaaaggtgcaggaatgacaagatggcca

SEQ ID NO: 647 - DNA, 37 bp, Artificial Sequence
  Synthetic polynucleotide 20 (R5DV1)
aatcacagcatagagccaataagacggtaaaaggcgt

SEQ ID NO: 648 - DNA, 29 bp, Artificial Sequence
  Synthetic polynucleotide 20 (R5DV1)
agcacgataggacgggtcacgagagtgag

SEQ ID NO: 649 - DNA, 35 bp, Artificial Sequence
  Synthetic polynucleotide 20 (R5DV1)
agaggtgccaacaactaggacaataagccgataaa

SEQ ID NO: 650 - DNA, 37 bp, Artificial Sequence
  Synthetic polynucleotide 20 (R5DV1)
agcgtcgaacaatagagacgtccagagaatggaccca

SEQ ID NO: 651 - DNA, 33 bp, Artificial Sequence
  Synthetic polynucleotide 20 (R5DV1)
atccaccggatagcaagagtagcggagataaga

SEQ ID NO: 652 - DNA, 37 bp, Artificial Sequence
  Synthetic polynucleotide 20 (R5DV1)
acgtcaggagatagcgaagtcacgaaatgagagagtc

SEQ ID NO: 653 - DNA, 37 bp, Artificial Sequence
  Synthetic polynucleotide 20 (R5DV1)
acgcggtagcacaataaagacgtacaaaagtacaaca

SEQ ID NO: 654 - DNA, 41 bp, Artificial Sequence
  Synthetic polynucleotide 20 (R5DV1)
aagtgagaacatcaaaacgataagcaggataaaaaggtaaa

SEQ ID NO: 655 - DNA, 29 bp, Artificial Sequence
  Synthetic polynucleotide 20 (R5DV1)
acgggatcgggacgctcgaaaactgacga

SEQ ID NO: 656 - DNA, 43 bp, Artificial Sequence
  Synthetic polynucleotide 20 (R5DV1)
actagaaaagtaagaacctagaaaaatagcggcatagaaaact

SEQ ID NO: 657 - DNA, 39 bp, Artificial Sequence
  Synthetic polynucleotide 20 (R5DV1)
aagggaatggcgaacataagaggaataggaaggtggcga

SEQ ID NO: 658 - DNA, 30 bp, Artificial Sequence
  Synthetic polynucleotide 20 (R5DV1)
agtggagcaataaaggaggtgggacggtca

SEQ ID NO: 659 - DNA, 36 bp, Artificial Sequence
  Synthetic polynucleotide 20 (R5DV1)
agagctagagaaatcgcaacgataccggaatcggga

SEQ ID NO: 660 - DNA, 30 bp, Artificial Sequence
  Synthetic polynucleotide 20 (R5DV1)
agtagaacaaatcagcggcgtaggacaagt

SEQ ID NO: 661 - DNA, 34 bp, Artificial Sequence
  Synthetic polynucleotide 20 (R5DV1)
ccgggaatccacgagtcgaaaaaataagacactc

SEQ ID NO: 662 - DNA, 28 bp, Artificial Sequence
  Synthetic polynucleotide 20 (R5DV1)
agagagtgcggaggctaaacgggtggaa

SEQ ID NO: 663 - DNA, 31 bp, Artificial Sequence
  Synthetic polynucleotide 20 (R5DV1)
agaactggaaagatccagagcatcgcagaat

SEQ ID NO: 664 - DNA, 26 bp, Artificial Sequence
  Synthetic polynucleotide 20 (R5DV1)
aagacgagtgcgcggatcaacggata

SEQ ID NO: 665 - DNA, 24 bp, Artificial Sequence
  Synthetic polynucleotide 20 (R5DV1)
accacctaagggcgtcgggcgata

SEQ ID NO: 666 - DNA, 25 bp, Artificial Sequence
  Synthetic polynucleotide 20 (R5DV1)
acggcagtaccgaaatgggctagcc

SEQ ID NO: 667 - DNA, 27 bp, Artificial Sequence
  Synthetic polynucleotide 20 (R5DV1)
acgggatgggagaatggaaccgtagga

SEQ ID NO: 668 - DNA, 34 bp, Artificial Sequence
  Synthetic polynucleotide 20 (R5DV1)
ccggtagagaaagtggaacactacggagaatgca

SEQ ID NO: 669 - DNA, 31 bp, Artificial Sequence
  Synthetic polynucleotide 20 (R5DV1)
acagtaggaaagctgacgggctaaggccggt

SEQ ID NO: 670 - DNA, 25 bp, Artificial Sequence
  Synthetic polynucleotide 20 (R5DV1)
aggacaagctccgcgcgtgaagata

SEQ ID NO: 671 - DNA, 25 bp, Artificial Sequence
  Synthetic polynucleotide 20 (R5DV1)
tcgcggagtagaggaataccgggcc

SEQ ID NO: 672 - DNA, 25 bp, Artificial Sequence
  Synthetic polynucleotide 20 (R5DV1)
atgacgggcactcaacggctgaaag

SEQ ID NO: 673 - DNA, 24 bp, Artificial Sequence
  Synthetic polynucleotide 20 (R5DV1)
agctgaagcaggtgcaggactggg

SEQ ID NO: 674 - DNA, 28 bp, Artificial Sequence
  Synthetic polynucleotide 20 (R5DV1)
aggagtagggacgatcacaggagcatgc

SEQ ID NO: 675 - DNA, 35 bp, Artificial Sequence
  Synthetic polynucleotide 21 (B6DV2)
agaagaaaagtggaacactaggcgggtgaacggga

SEQ ID NO: 676 - DNA, 33 bp, Artificial Sequence
  Synthetic polynucleotide 21 (B6DV2)
tgaaaaggatagagcggtgcgaaagtcaagaaa

SEQ ID NO: 677 - DNA, 40 bp, Artificial Sequence
  Synthetic polynucleotide 21 (B6DV2)
taggagaactaacgaagtgggagagactaaagacgtcaga

SEQ ID NO: 678 - DNA, 36 bp, Artificial Sequence
  Synthetic polynucleotide 21 (B6DV2)
agatcaaaaggtcggaggcaatggagaagtgcagca

SEQ ID NO: 679 - DNA, 26 bp, Artificial Sequence
  Synthetic polynucleotide 21 (B6DV2)
atcagaccgtccggcgctgaaaacca

SEQ ID NO: 680 - DNA, 34 bp, Artificial Sequence
  Synthetic polynucleotide 21 (B6DV2)
atgaagacagatccaagagctgagcagctagcga

SEQ ID NO: 681 - DNA, 32 bp, Artificial Sequence
  Synthetic polynucleotide 21 (B6DV2)
agagtggcgagcgtaaagcagtagggaggtaa

SEQ ID NO: 682 - DNA, 32 bp, Artificial Sequence
  Synthetic polynucleotide 21 (B6DV2)
aagaactacaggcctcagacaatccagacgta

SEQ ID NO: 683 - DNA, 26 bp, Artificial Sequence
  Synthetic polynucleotide 21 (B6DV2)
agagggatacagccgtcagggaggta

SEQ ID NO: 684 - DNA, 29 bp, Artificial Sequence
  Synthetic polynucleotide 21 (B6DV2)
agagaatgaaaggatgcgcccatgagcca

SEQ ID NO: 685 - DNA, 33 bp, Artificial Sequence
  Synthetic polynucleotide 21 (B6DV2)
actgggagggctaaaaaactagaagagtgaaaa

SEQ ID NO: 686 - DNA, 32 bp, Artificial Sequence
  Synthetic polynucleotide 21 (B6DV2)
actggcaccgtgagaaaatagcacaatacgaa

SEQ ID NO: 687 - DNA, 26 bp, Artificial Sequence
  Synthetic polynucleotide 21 (B6DV2)
aggtcgagaaatcgggaaagtgcgga

SEQ ID NO: 688 - DNA, 35 bp, Artificial Sequence
  Synthetic polynucleotide 21 (B6DV2)
agtggaccactagcgagatcaacagagtagggaca

SEQ ID NO: 689 - DNA, 35 bp, Artificial Sequence
  Synthetic polynucleotide 21 (B6DV2)
tcgaagaataaggcagaatgcgacagtacgggaga

SEQ ID NO: 690 - DNA, 28 bp, Artificial Sequence
  Synthetic polynucleotide 21 (B6DV2)
tcgaaagactgcgagcgtgacgagatga

SEQ ID NO: 691 - DNA, 38 bp, Artificial Sequence
  Synthetic polynucleotide 21 (B6DV2)
agaggtaaaaaactgaaaacatgaggcggtgaaaggga

SEQ ID NO: 692 - DNA, 28 bp, Artificial Sequence
  Synthetic polynucleotide 21 (B6DV2)
ataaccaggtgggacacagtgacgggca

SEQ ID NO: 693 - DNA, 30 bp, Artificial Sequence
  Synthetic polynucleotide 21 (B6DV2)
tggcgggcctcaagagatgcacgactgagc

SEQ ID NO: 694 - DNA, 40 bp, Artificial Sequence
  Synthetic polynucleotide 21 (B6DV2)
aggtgagaaggtacacagatacgagaatggaacggctcaa

SEQ ID NO: 695 - DNA, 33 bp, Artificial Sequence
  Synthetic polynucleotide 21 (B6DV2)
aacaataagaaggtcggcccgtgagaccaggta

SEQ ID NO: 696 - DNA, 26 bp, Artificial Sequence
  Synthetic polynucleotide 21 (B6DV2)
agagagctggaggacccgcggaggtg

SEQ ID NO: 697 - DNA, 34 bp, Artificial Sequence
  Synthetic polynucleotide 21 (B6DV2)
agcgggtcaaaaaatcgagagataaggagagtga

SEQ ID NO: 698 - DNA, 39 bp, Artificial Sequence
  Synthetic polynucleotide 21 (B6DV2)
acgggtgaagacagtagaaaaatgagagaaatccggcaa

SEQ ID NO: 699 - DNA, 34 bp, Artificial Sequence
  Synthetic polynucleotide 21 (B6DV2)
atagcaggactggacgcgtacgaaagagtggcaa

SEQ ID NO: 700 - DNA, 26 bp, Artificial Sequence
  Synthetic polynucleotide 21 (B6DV2)
aataagcggatggcgagatggcgggc

SEQ ID NO: 701 - DNA, 37 bp, Artificial Sequence
  Synthetic polynucleotide 21 (B6DV2)
tcggcgagatagcaagatgaagacgtaagaacggtaa

SEQ ID NO: 702 - DNA, 40 bp, Artificial Sequence
  Synthetic polynucleotide 21 (B6DV2)
aaggctcaagagatgaacaaataaagagatacgcggctaa

SEQ ID NO: 703 - DNA, 37 bp, Artificial Sequence
  Synthetic polynucleotide 21 (B6DV2)
aagacctaaaaggataagaaactcaaggcagtgacga

SEQ ID NO: 704 - DNA, 34 bp, Artificial Sequence
  Synthetic polynucleotide 21 (B6DV2)
aacgtgcaagcagtaacagaatgagaaaggatga

SEQ ID NO: 705 - DNA, 39 bp, Artificial Sequence
  Synthetic polynucleotide 21 (B6DV2)
acaccgtggcgggataggagagtggagaaatggaagaat

SEQ ID NO: 706 - DNA, 40 bp, Artificial Sequence
  Synthetic polynucleotide 21 (B6DV2)
acaagaataagaacggtcggacgcataaacaggtaagcca

SEQ ID NO: 707 - DNA, 35 bp, Artificial Sequence
  Synthetic polynucleotide 21 (B6DV2)
ataagagaggtcagcacacgtacgaaggaagatct

SEQ ID NO: 708 - DNA, 32 bp, Artificial Sequence
  Synthetic polynucleotide 22 (G6DV2)
agacggatagaagcatggcagaggatcaggga

SEQ ID NO: 709 - DNA, 36 bp, Artificial Sequence
  Synthetic polynucleotide 22 (G6DV2)
agtcaaacgaatacaaagatagccaactaggaccaa

SEQ ID NO: 710 - DNA, 33 bp, Artificial Sequence
  Synthetic polynucleotide 22 (G6DV2)
tcagagactgagaagcgtaagcgaagtacgaca

SEQ ID NO: 711 - DNA, 32 bp, Artificial Sequence
  Synthetic polynucleotide 22 (G6DV2)
atcggaaagtcgagggatgcgagagatacaaa

SEQ ID NO: 712 - DNA, 30 bp, Artificial Sequence
  Synthetic polynucleotide 22 (G6DV2)
agtaagagggtagaaggcagtcgggagcca

SEQ ID NO: 713 - DNA, 36 bp, Artificial Sequence
  Synthetic polynucleotide 22 (G6DV2)
tccgggagctaacaaaataaagaactggcagaggct

SEQ ID NO: 714 - DNA, 27 bp, Artificial Sequence
  Synthetic polynucleotide 22 (G6DV2)
agcgctccgggagataggaaggatgac

SEQ ID NO: 715 - DNA, 32 bp, Artificial Sequence
  Synthetic polynucleotide 22 (G6DV2)
aggccgtccgaaagtacggaaatcaaaaagtg

SEQ ID NO: 716 - DNA, 33 bp, Artificial Sequence
  Synthetic polynucleotide 22 (G6DV2)
aggcactcagagagtgaaagcgtaagaacgggt

SEQ ID NO: 717 - DNA, 24 bp, Artificial Sequence
  Synthetic polynucleotide 22 (G6DV2)
agggagctaggcgggtaggccaca

SEQ ID NO: 718 - DNA, 32 bp, Artificial Sequence
  Synthetic polynucleotide 22 (G6DV2)
tcacgggataaagagatgacaagcgtgaagga

SEQ ID NO: 719 - DNA, 25 bp, Artificial Sequence
  Synthetic polynucleotide 22 (G6DV2)
atccacggagtgcgcagacgtccaa

SEQ ID NO: 720 - DNA, 24 bp, Artificial Sequence
  Synthetic polynucleotide 22 (G6DV2)
aggtccacaactccgccgggtaca

SEQ ID NO: 721 - DNA, 26 bp, Artificial Sequence
  Synthetic polynucleotide 22 (G6DV2)
acggtgaagcaaatcacgggctcgaa

SEQ ID NO: 722 - DNA, 32 bp, Artificial Sequence
  Synthetic polynucleotide 22 (G6DV2)
aaggtggcgagatgggaccatgaaagaatcga

SEQ ID NO: 723 - DNA, 37 bp, Artificial Sequence
  Synthetic polynucleotide 22 (G6DV2)
agggtaagacgatccagagatgagcccataacaaggt

SEQ ID NO: 724 - DNA, 42 bp, Artificial Sequence
  Synthetic polynucleotide 22 (G6DV2)
aacaacctaacagaagtaccagaatagagcaactgaaaaagt

SEQ ID NO: 725 - DNA, 31 bp, Artificial Sequence
  Synthetic polynucleotide 22 (G6DV2)
acagcactggcaacgtccaaggctcgcggcc

SEQ ID NO: 726 - DNA, 28 bp, Artificial Sequence
  Synthetic polynucleotide 22 (G6DV2)
tgcgcgcgtgcgccgaaataaggacaat

SEQ ID NO: 727 - DNA, 36 bp, Artificial Sequence
  Synthetic polynucleotide 22 (G6DV2)
aacgacctaggaccgaatacaaaagctaacagactc

SEQ ID NO: 728 - DNA, 31 bp, Artificial Sequence
  Synthetic polynucleotide 22 (G6DV2)
agagcatccaacgctgagccagtcagaaggt

SEQ ID NO: 729 - DNA, 41 bp, Artificial Sequence
  Synthetic polynucleotide 22 (G6DV2)
agagaagtaagcgaaataaagaaaataaagaaatgccgcga

SEQ ID NO: 730 - DNA, 33 bp, Artificial Sequence
  Synthetic polynucleotide 22 (G6DV2)
tcggaaggtgaagaactaccaggctacggagaa

SEQ ID NO: 731 - DNA, 42 bp, Artificial Sequence
  Synthetic polynucleotide 22 (G6DV2)
tgaaagggtaagagggtaaggacatacaagaataaagaagct

SEQ ID NO: 732 - DNA, 33 bp, Artificial Sequence
  Synthetic polynucleotide 22 (G6DV2)
aggacaataaacgccctaaaaccgcggagagaa

SEQ ID NO: 733 - DNA, 25 bp, Artificial Sequence
  Synthetic polynucleotide 22 (G6DV2)
tagcccgatacaagcgtccgggcat

SEQ ID NO: 734 - DNA, 37 bp, Artificial Sequence
  Synthetic polynucleotide 22 (G6DV2)
agaaacctggagaaataggacaggtgaaaggctgggc

SEQ ID NO: 735 - DNA, 35 bp, Artificial Sequence
  Synthetic polynucleotide 22 (G6DV2)
acatgaccgactggagagcatggaaacatacaccc

SEQ ID NO: 736 - DNA, 30 bp, Artificial Sequence
  Synthetic polynucleotide 22 (G6DV2)
agtcaaaaagtcgaggaatagccgggtggc

SEQ ID NO: 737 - DNA, 37 bp, Artificial Sequence
  Synthetic polynucleotide 22 (G6DV2)
acggtaacaaaatcaagaaaataccggagggtgcaga

SEQ ID NO: 738 - DNA, 31 bp, Artificial Sequence
  Synthetic polynucleotide 22 (G6DV2)
agggtacgaccggtacaggacctaagaacga

SEQ ID NO: 739 - DNA, 31 bp, Artificial Sequence
  Synthetic polynucleotide 22 (G6DV2)
tggcgagagtcgggccgtgaggacagtaagg

SEQ ID NO: 740 - DNA, 37 bp, Artificial Sequence
  Synthetic polynucleotide 22 (G6DV2)
acgtggaggaagtagcagaatagcgggatagccagct

SEQ ID NO: 741 - DNA, 31 bp, Artificial Sequence
  Synthetic polynucleotide 22 (G6DV2)
acgaagaaatgggcaagtgcggagaagatct

SEQ ID NO: 742 - DNA, 32 bp, Artificial Sequence
  Synthetic polynucleotide 23 (Y6DV2)
agatcgaaaaatagggaggtggccggctgcga

SEQ ID NO: 743 - DNA, 30 bp, Artificial Sequence
  Synthetic polynucleotide 23 (Y6DV2)
aagtcgggcgggtgaaagcaactaaaagga

SEQ ID NO: 744 - DNA, 37 bp, Artificial Sequence
  Synthetic polynucleotide 23 (Y6DV2)
tcgaagcatgaaagagtaggaaagtggaagaatgaga

SEQ ID NO: 745 - DNA, 45 bp, Artificial Sequence
  Synthetic polynucleotide 23 (Y6DV2)
agataacgaaataaggaaggtaaaacggtggagagatagaggaca

SEQ ID NO: 746 - DNA, 38 bp, Artificial Sequence
  Synthetic polynucleotide 23 (Y6DV2)
taacaaagtggaaacactcaagagctaagggaactaga

SEQ ID NO: 747 - DNA, 31 bp, Artificial Sequence
  Synthetic polynucleotide 23 (Y6DV2)
agcataacggaatggctagcatcgggagagt

SEQ ID NO: 748 - DNA, 30 bp, Artificial Sequence
  Synthetic polynucleotide 23 (Y6DV2)
agcggcatgaagcgatagggaacgctgaca

SEQ ID NO: 749 - DNA, 38 bp, Artificial Sequence
  Synthetic polynucleotide 23 (Y6DV2)
agaaataaccgaatcgggaaatcaaaccatagaagact

SEQ ID NO: 750 - DNA, 34 bp, Artificial Sequence
  Synthetic polynucleotide 23 (Y6DV2)
agacgggtaacagaatggaggcaataggaaacgt

SEQ ID NO: 751 - DNA, 32 bp, Artificial Sequence
  Synthetic polynucleotide 23 (Y6DV2)
aagacactaacgggataccacgagtgacaaga

SEQ ID NO: 752 - DNA, 33 bp, Artificial Sequence
  Synthetic polynucleotide 23 (Y6DV2)
tcggaggatggcaccatgaaaagatagagagct

SEQ ID NO: 753 - DNA, 37 bp, Artificial Sequence
  Synthetic polynucleotide 23 (Y6DV2)
agaagggtacgggaatagaaaaatgcaaagctagggc

SEQ ID NO: 754 - DNA, 34 bp, Artificial Sequence
  Synthetic polynucleotide 23 (Y6DV2)
cgtcgaccggtgagaagggtaaaaagggtgacaa

SEQ ID NO: 755 - DNA, 35 bp, Artificial Sequence
  Synthetic polynucleotide 23 (Y6DV2)
aatgagagaataaccagatagggacgtgaaaggct

SEQ ID NO: 756 - DNA, 33 bp, Artificial Sequence
  Synthetic polynucleotide 23 (Y6DV2)
aggcacctggagacaatgaggcagtacacgcgt

SEQ ID NO: 757 - DNA, 37 bp, Artificial Sequence
  Synthetic polynucleotide 23 (Y6DV2)
accaagatccagagaatcagacggtgagacactggac

SEQ ID NO: 758 - DNA, 36 bp, Artificial Sequence
  Synthetic polynucleotide 23 (Y6DV2)
accataagaagatgaggaggtgagggacatgaaaca

SEQ ID NO: 759 - DNA, 37 bp, Artificial Sequence
  Synthetic polynucleotide 23 (Y6DV2)
ataacaacaataaccacataagggcctcgaaacgtgg

SEQ ID NO: 760 - DNA, 32 bp, Artificial Sequence
  Synthetic polynucleotide 23 (Y6DV2)
agagcatacagccggtgcaaaagtgagacgga

SEQ ID NO: 761 - DNA, 31 bp, Artificial Sequence
  Synthetic polynucleotide 23 (Y6DV2)
tgcgaaaaatgaacaggctgggacgataccc

SEQ ID NO: 762 - DNA, 28 bp, Artificial Sequence
  Synthetic polynucleotide 23 (Y6DV2)
agaatgccaagatggcggcctgccggga

SEQ ID NO: 763 - DNA, 34 bp, Artificial Sequence
  Synthetic polynucleotide 23 (Y6DV2)
tcaccgagtagccaacctgacaaaaagtgcggaa

SEQ ID NO: 764 - DNA, 29 bp, Artificial Sequence
  Synthetic polynucleotide 23 (Y6DV2)
atacgggcatgagaaggtggagcactcag

SEQ ID NO: 765 - DNA, 34 bp, Artificial Sequence
  Synthetic polynucleotide 23 (Y6DV2)
acgatgaagacgatacggacgtaccaaaatggaa

SEQ ID NO: 766 - DNA, 34 bp, Artificial Sequence
  Synthetic polynucleotide 23 (Y6DV2)
aacaatgggagcgtaagcaaaatcagacggtaga

SEQ ID NO: 767 - DNA, 33 bp, Artificial Sequence
  Synthetic polynucleotide 23 (Y6DV2)
acggtaaagagatgcacaagatgaagagcatca

SEQ ID NO: 768 - DNA, 25 bp, Artificial Sequence
  Synthetic polynucleotide 23 (Y6DV2)
acacatgacagcccgcgggagtagg

SEQ ID NO: 769 - DNA, 31 bp, Artificial Sequence
  Synthetic polynucleotide 23 (Y6DV2)
cggagtaagcgggtaacgaggtgagcacata

SEQ ID NO: 770 - DNA, 35 bp, Artificial Sequence
  Synthetic polynucleotide 23 (Y6DV2)
aaaaggtcgcaaagtacgaaggtaagggagtggag

SEQ ID NO: 771 - DNA, 27 bp, Artificial Sequence
  Synthetic polynucleotide 23 (Y6DV2)
agagtgaagggcctggagggagtcgga

SEQ ID NO: 772 - DNA, 35 bp, Artificial Sequence
  Synthetic polynucleotide 23 (Y6DV2)
acaatgccgacgtaagcaagtcaaacgctaaggca

SEQ ID NO: 773 - DNA, 28 bp, Artificial Sequence
  Synthetic polynucleotide 23 (Y6DV2)
agataacggaatgcgaaactggcgccct

SEQ ID NO: 774 - DNA, 37 bp, Artificial Sequence
  Synthetic polynucleotide 23 (Y6DV2)
aagaacgtaggaccataacaaggtagaaaaaagatct

SEQ ID NO: 775 - DNA, 37 bp, Artificial Sequence
  Synthetic polynucleotide 24 (R6DV2)
agatggaaagctgagagaaagtcagaagatcacagac

SEQ ID NO: 776 - DNA, 30 bp, Artificial Sequence
  Synthetic polynucleotide 24 (R6DV2)
tcaggaggatcgggcagtagacacgtaaga

SEQ ID NO: 777 - DNA, 32 bp, Artificial Sequence
  Synthetic polynucleotide 24 (R6DV2)
aggtagaggccaatgacaggctgaagaggtga

SEQ ID NO: 778 - DNA, 30 bp, Artificial Sequence
  Synthetic polynucleotide 24 (R6DV2)
aggcctaaaagaatacgcgggtcaggaaca

SEQ ID NO: 779 - DNA, 28 bp, Artificial Sequence
  Synthetic polynucleotide 24 (R6DV2)
atgcgaagcctgggacgatcgggagata

SEQ ID NO: 780 - DNA, 32 bp, Artificial Sequence
  Synthetic polynucleotide 24 (R6DV2)
aaagggatagagaaatggccgaatagaccggt

SEQ ID NO: 781 - DNA, 24 bp, Artificial Sequence
  Synthetic polynucleotide 24 (R6DV2)
acggcgcgctagcgataaggaact

SEQ ID NO: 782 - DNA, 32 bp, Artificial Sequence
  Synthetic polynucleotide 24 (R6DV2)
agacgggtaaaaccgtaggaagctcagaaaca

SEQ ID NO: 783 - DNA, 36 bp, Artificial Sequence
  Synthetic polynucleotide 24 (R6DV2)
taacgaaagctacagcaggtaagcaagctaagaacc

SEQ ID NO: 784 - DNA, 33 bp, Artificial Sequence
  Synthetic polynucleotide 24 (R6DV2)
tgagcaggataacgcagtaaggacactacggga

SEQ ID NO: 785 - DNA, 31 bp, Artificial Sequence
  Synthetic polynucleotide 24 (R6DV2)
actaagccagatgaccgggtacgaacgtcaa

SEQ ID NO: 786 - DNA, 27 bp, Artificial Sequence
  Synthetic polynucleotide 24 (R6DV2)
acgaatagcaaaggtgcgggacgtggc

SEQ ID NO: 787 - DNA, 38 bp, Artificial Sequence
  Synthetic polynucleotide 24 (R6DV2)
cgaagatagaaaacatacccaaatgccggaatgggaga

SEQ ID NO: 788 - DNA, 34 bp, Artificial Sequence
  Synthetic polynucleotide 24 (R6DV2)
atgcgcaagtaaaaacataggaacagtaaggcaa

SEQ ID NO: 789 - DNA, 32 bp, Artificial Sequence
  Synthetic polynucleotide 24 (R6DV2)
tcgggaggtgaaagggaatgagacaactaaca

SEQ ID NO: 790 - DNA, 36 bp, Artificial Sequence
  Synthetic polynucleotide 24 (R6DV2)
aggtgggaaaaatccagcagtaaacagcatagggca

SEQ ID NO: 791 - DNA, 44 bp, Artificial Sequence
  Synthetic polynucleotide 24 (R6DV2)
atgagaaggtagaacaataaagacgatgaggaagtaaaaacggg

SEQ ID NO: 792 - DNA, 35 bp, Artificial Sequence
  Synthetic polynucleotide 24 (R6DV2)
tgggcaaatgacacgatgaaaaagtaacggagtaa

SEQ ID NO: 793 - DNA, 36 bp, Artificial Sequence
  Synthetic polynucleotide 24 (R6DV2)
acccactcagaaactggaaaaagtcgaaacatggga

SEQ ID NO: 794 - DNA, 36 bp, Artificial Sequence
  Synthetic polynucleotide 24 (R6DV2)
agaatacaccacatccagcgaataacgcgactccca

SEQ ID NO: 795 - DNA, 36 bp, Artificial Sequence
  Synthetic polynucleotide 24 (R6DV2)
accaatagacgagtgaagagatggaagccctggcga

SEQ ID NO: 796 - DNA, 34 bp, Artificial Sequence
  Synthetic polynucleotide 24 (R6DV2)
acatggagacaataggagggtcaaggacgtggac

SEQ ID NO: 797 - DNA, 27 bp, Artificial Sequence
  Synthetic polynucleotide 24 (R6DV2)
acgtacagcggtaacggcctcagcagg

SEQ ID NO: 798 - DNA, 28 bp, Artificial Sequence
  Synthetic polynucleotide 24 (R6DV2)
tgggaggctacgacgaatggaggagtgc

SEQ ID NO: 799 - DNA, 26 bp, Artificial Sequence
  Synthetic polynucleotide 24 (R6DV2)
agcaataggcgggatagcagcctgca

SEQ ID NO: 800 - DNA, 34 bp, Artificial Sequence
  Synthetic polynucleotide 24 (R6DV2)
aggatcggcaactgagaaagtgaagagaatgcca

SEQ ID NO: 801 - DNA, 29 bp, Artificial Sequence
  Synthetic polynucleotide 24 (R6DV2)
accctgcaagcgtaaaaagcgtgaaggcg

SEQ ID NO: 802 - DNA, 34 bp, Artificial Sequence
  Synthetic polynucleotide 24 (R6DV2)
tccagaagtacgcacactggacaaatagacgaat

SEQ ID NO: 803 - DNA, 26 bp, Artificial Sequence
  Synthetic polynucleotide 24 (R6DV2)
aagggcctcaaaagcatcgcgaggat

SEQ ID NO: 804 - DNA, 29 bp, Artificial Sequence
  Synthetic polynucleotide 24 (R6DV2)
aagaaagtgcgggagtaacaacctcggga

SEQ ID NO: 805 - DNA, 33 bp, Artificial Sequence
  Synthetic polynucleotide 24 (R6DV2)
agtcggaaagtagaaaggtggaccgataacccg

SEQ ID NO: 806 - DNA, 26 bp, Artificial Sequence
  Synthetic polynucleotide 24 (R6DV2)
cgggcacaataaacgaactgcgagca

SEQ ID NO: 807 - DNA, 35 bp, Artificial Sequence
  Synthetic polynucleotide 24 (R6DV2)
tcagagaggtgcagaagataccacgagtagaggca

SEQ ID NO: 808 - DNA, 42 bp, Artificial Sequence
  Synthetic polynucleotide 24 (R6DV2)
tgggaacgtgaaaacaataacgcaagtcaggacgagagatct

SEQ ID NO: 809 - DNA, 14 bp, Artificial Sequence
  Synthetic Polynucleotide
ctgtctcatctctt

SEQ ID NO: 810 - DNA, 26 bp, Artificial Sequence
  Synthetic Polynucleotide
ctgtctcatctcttgctgcatcctgt

SEQ ID NO: 811 - DNA, 38 bp, Artificial Sequence
  Synthetic Polynucleotide
ctgtctcatctcttgctgcatcctgtcggttcacgttg

SEQ ID NO: 812 - DNA, 36 bp, Artificial Sequence
  Synthetic Polynucleotide
ctgtctcatcttgctgcatcctgtcggttcacgttg

SEQ ID NO: 813 - DNA, 36 bp, Artificial Sequence
  Synthetic Polynucleotide
ctgtctcattttgctgcatcctgtccgttcacgttg